(19) United States
(12) Patent Application Publication
(10) Pub. No.: US 2007/0099929 A1
(43) Pub. Date: May 3, 2007

(54) Substituted thiophenes

(75) Inventors: Kai Thede, Wuppertal (DE); Tobias Wunberg, Hinterbruehl (AT); Timothy Lowinger, Guilford, CT (US); Diana Koletzki, Brussels (BE); Andreas Urban, Hattingen (DE); Judith Baumeister, Mechelen (BE); Kerstin Henninger, Wuppertal (DE)

Correspondence Address: MORRISON & FOERSTER LLP, 12531 HIGH BLUFF DRIVE, SUITE 100, SAN DIEGO, CA, 92130-2040, US

(73) Assignee: AiCuris GmbH & Co. KG, Wuppertal (DE)

(21) Appl. No.: 11/455,253

(22) Filed: Jun. 16, 2006

Related U.S. Application Data
(63) Continuation of application No. PCT/EP04/14335, filed on Dec. 16, 2004.

(30) Foreign Application Priority Data
Dec. 19, 2003 (DE) 103 59 791.3

(51) Int. Cl. A61K 31/496; A61K 31/453; A61K 31/4436; A61K 31/4025; A61K 31/381; C07D 409/02

(52) U.S. Cl. 514/252.13; 514/326; 514/342; 514/422; 514/444; 544/379; 546/207; 548/537; 549/59; 549/69

(57) ABSTRACT

The invention relates to substituted thiophenes and processes for their preparation, their use for the treatment and/or prophylaxis of diseases, and their use for the production of medicaments for the treatment and/or prophylaxis of diseases, especially for use as antiviral agents, in particular for hepatitis C viruses.

### CROSS REFERENCE TO RELATED APPLICATIONS

This application is a continuation of pending International Patent Application PCT/EP2004/014335 filed on Dec. 16, 2004 designating U.S., which claims priority from German Patent Application DE 103 59 791.3 filed on Dec. 19, 2003.

### BACKGROUND OF THE INVENTION

The invention relates to substituted thiophenes and processes for their preparation, their use for the treatment and/or prophylaxis of diseases, and their use for producing medicaments for the treatment and/or prophylaxis of diseases, especially for use as antiviral agents, in particular for hepatitis C viruses.

Infections with the hepatitis C virus (HCV) are the main cause for non-A/non-B hepatitis illnesses around the world. It is estimated that about 170 million people around the world are infected with the virus. This leads in a high percentage of virus carriers to a chronic hepatitis C illness. This group of infected people is at high risk of subsequently dying of life-threatening hepatic disorders such as cirrhosis of the liver, hepatocellular carcinoma or terminal hepatic failure. Hepatitis C infection is one of the most common reasons for a liver transplant. The mechanisms leading to the persistence of the viral infection and to the high rate of serious hepatic disorders resulting therefrom have not yet been completely elucidated. It is unknown how the virus interacts with the human immune system and overcomes immune defenses. The part played by cellular and humoral immune responses in the protection against a HCV infection is not yet understood. It has been reported that immunoglobulins have been employed for prophylactic protection against transfusion-related viral hepatitis; however, the use of immunoglobulins for this purpose is not currently recommended by the Center for Disease Control. The absence of an efficient immune response has to date impeded the establishment of a vaccine and likewise of a prophylaxis which could be employed after contact with the virus. In the near future, therefore, mainly antiviral principles will play a part in controlling the hepatitis C virus.

Various clinical studies have investigated substances with the aim of an effective therapy of HCV infections in patients with chronic hepatitis. In these studies interferon alpha (IFN-α), given alone or in combination with other antiviral drugs, was employed. These investigations have shown that a considerable number of patients does not respond to this therapy, and that many of those in whom interferon alpha shows an effect suffer relapses after discontinuation of the substance.

Until recently, treatment with interferon (IFN) was the only type of therapy with clinically demonstrated efficacy for chronic hepatitis C illness. However, the proportion of patients in whom the therapy is permanently successful is low. Interferon therapy is always associated with serious side effects (e.g., leukopenia, thrombopenia, retinopathy, thyroiditis, acute pancreatitis, depression) which considerably impair the patients' quality of life. The combination of interferon with ribavirin was recently approved. This combination therapy leads to an improved efficacy, but does not improve the side effect profile associated with IFN and, moreover, side effects (e.g., hemolytic anemia) are also associated with ribavirin. These adverse side effects can be at least partially ameliorated by the use of PEGylated forms of IFN such as PEG-Intron® or Pegasys®. Irrespective of this, there is a great need for orally applicable antiviral active agents with which the limitations of the currently established forms of therapy can be overcome (S.-L. Tan et al., Nature Rev. Drug Discov. 2002, 1, 867-881).

Hepatitis C virus (HCV) is the only representative of the genus Hepacivirus within the family of Flaviviridae. At least 6 genotypes and a large number of subtypes are distinguished. The virus is surrounded by an envelope and has a positive single strand of viral RNA as genome. The length of the viral RNA genome is about 9500 nucleotides. Replication of the viral genome and translation into protein takes place with the aid of RNA structures which are located at the start and end of the genome (5′ untranslated region, 3′ untranslated region). The genome has a single reading frame (open reading frame, ORF) which codes for a polyprotein (about 3000 amino acids). Structural and non-structural (NS) proteins are cleaved from this in an infected cell by viral or host cell enzymes. HCV codes for a capsid protein (c) and two envelope proteins (E1 and E2). A small protein (p7) might be a so-called viroporin which is essential for the infectiousness of the mature virus particle. The mature NS proteins include the proteins NS2, NS3, NS4A, NS4B, NS5A and NS5B. Two viral proteases are responsible for their separation from the polyprotein. The enzyme designated NS2/3 protease cleaves, in a manner which is only poorly characterized as yet, the NS2-NS3 cleavage site. The second protease (NS3 protease) is a serine protease which is present in the N-terminal part of the NS3 protein. It catalyses all cleavages of the polyprotein downstream of NS3, i.e. NS3-NS4A proteolysis as well as cleavages at the NS4A-NS4B, NS4B-NS5A, NS5A-NS5B sites.

The NS4A protein probably has a diversity of functions, for example as cofactor of the NS3 protease and possibly in the membrane localization of NS3 and other NS proteins. The formation of a complex between NS3 and NS4A is evidently a basic requirement for protein processing and increases the proteolytic activities in relation to all cleavage sites. The NS3 protein additionally has NTPase and helicase activity. NS5B is an RNA-dependent RNA polymerase which is crucially involved in HCV replication. Very little is known about the functions of the NS4B and NS5A proteins. NS5A is suggested to be involved in clinical resistance to interferon.

Viruses closely related to hepatitis C, such as, for example, the GBV B virus which infects new world monkeys, or the BVDV (bovine viral diarrhea virus) are frequently used as model viruses in order to investigate certain aspects of the viral lifecycle.

### SUMMARY OF THE INVENTION

One object of the present invention is therefore to provide novel compounds having the same or improved antiviral action for the treatment and/or prophylaxis of viral infectious diseases in humans and animals, in particular of hepatitis C and its sequelae.

Structurally similar compounds are described for example in WO 02/100851, WO 04/052879 and WO 04/052885 for the treatment and/or prophylaxis of flavivirus infections such as, for example, hepatitis C, in WO 00/027823 as gastrin and/or cholecystokinin receptors for the treatment of cancer and disorders of the central nervous system, in WO 92/010094 in combination with aryloxy acetic acid derivatives as herbicides and in EP-A 423 523 as herbicides.

Laval Chan, et al., Bioorg. Med. Chem. Lett. 2004, 14, 793-796 and Laval Chan, et al., Bioorg. Med. Chem. Lett. 2004, 14, 797-800 describe substituted thiophene-2-carboxylic acids as potent HCV inhibitors.

It has surprisingly been found that the substituted thiophenes described in the present invention have high antiviral activity.

The invention relates to compounds of formula

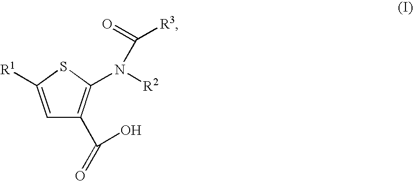

in which

R¹ represents (C₃-C₆)-alkyl, (C₃-C₇)-cycloalkyl, 5- to 7-membered heterocyclyl, phenyl or 5- or 6-membered heteroaryl,
whereby phenyl, cycloalkyl, heterocyclyl and heteroaryl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, aminothiocarbonyl, hydroxymethyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkylsulfoxyl, (C₁-C₆)-alkoxycarbonyl, (C₁-C₆)-alkylaminocarbonyl, (C₁-C₆)-alkylaminothiocarbonyl and (C₁-C₆)-alkylcarbonylamino,

R² represents (C₁-C₆)-alkyl, (C₃-C₇)-cycloalkyl, 5- to 7-membered heterocyclyl or benzyl,
whereby alkyl, cycloalkyl, heterocyclyl and benzyl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkylsulfoxyl, (C₁-C₆)-alkoxycarbonyl, (C₁-C₆)-alkylaminocarbonyl, (C₁-C₆)-alkylcarbonylamino, 5- to 7-membered heterocyclyl, optionally alkyl-substituted (C₃-C₇)-cycloalkylaminocarbonyl and optionally alkyl-substituted 5- to 7-membered heterocyclylcarbonyl,
in which alkyl may be substituted with 1 to 2 substituents, whereby the substituents are selected independently of one another from the group consisting of hydroxy, amino, (C₁-C₆)-alkylamino, (C₁-C₆)-alkylsulfoxyl and (C₁-C₆)-alkoxycarbonyl,

R³ represents (C₃-C₇)-cycloalkyl, 5- to 7-membered heterocyclyl, (C₆-C₁₀)-aryl, 5- to 7-membered heteroaryl, —CH₂—R⁴ or —CH₂—CH₂—R⁵,
whereby cycloalkyl, heterocyclyl, aryl and heteroaryl may be substituted with 1 to 3 substituents, whereby the substituents are selected independently of one another from the group consisting of halogen, cyano, hydroxy, amino, Nitro, trifluoromethyl, trifluoromethoxy, hydroxycarbonyl, aminocarbonyl, (C₁-C₆)-alkyl, (C₁-C₆)-alkylthio, (C₁-C₆)-alkylcarbonyl, (C₁-C₆)-alkylsulfonyl, (C₁-C₆)-alkoxycarbonyl, (C₆-C₁₀)-aryloxycarbonyl, (C₁-C₆)-alkylaminocarbonyl, (C₁-C₆)-alkylcarbonylamino, —OR⁶ and —NR⁷R⁸,
in which alkyl may be substituted with 1 to 3 substituents, whereby the substituents are selected independently of one another from the group consisting of halogen, hydroxy, amino, hydroxycarbonyl, aminocarbonyl, (C₁-C₆)-alkoxy, (C₁-C₆)-alkylamino, (C₁-C₆)-alkylthio, phenyl, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
in which phenyl in turn may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
   whereby
   R⁶ and R⁷ represent independently of one another (C₁-C₆)-alkyl, (C₃-C₇)-cycloalkyl, 5- to 7-membered heterocyclyl, benzyl, (C₆-C₁₀)-aryl or 5- or 6-membered heteroaryl,
   whereby alkyl, cycloalkyl, heterocyclyl, benzyl, aryl and heteroaryl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
   R⁸ represents hydrogen, (C₁-C₆)-alkyl or (C₃-C₇)-cycloalkyl,
   or
   R⁷ and R⁸ form a 5- to 7-membered heterocycle with the nitrogen atom to which they are bonded,

R⁴ and R⁵ represent, independently of one another, (C₃-C₇)-cycloalkyl, 5- to 7-membered heterocyclyl, (C₆-C₁₀)-aryl or 5- to 7-membered heteroaryl,
whereby cycloalkyl, heterocyclyl, aryl and heteroaryl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
and the salts thereof, the solvates thereof and the solvates of the salts thereof.

Compounds according to the invention are the compounds of formula (I) and the salts, solvates and solvates of the salts thereof, compounds mentioned below as exemplary embodiment(s), and the salts, solvates and solvates of the salts thereof, insofar as the compounds encompassed by formula (I) and mentioned below are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure substituents can be isolated in a known manner from such mixtures of enantiomers and/or diastereomers.

Where the compounds of the invention can occur in tautomeric forms, the present invention encompasses all tautomeric forms.

Salts preferred for the purposes of the present invention are physiologically acceptable salts of the compounds of the invention. However, salts which are themselves unsuitable for pharmaceutical applications but can be used for example for isolating or purifying the compounds of the invention are also encompassed.

Physiologically acceptable salts of the compounds of the invention include acid addition salts of mineral acids, carboxylic acids and sulfonic acids, e.g., salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, for example and preferably, alkali metal salts (e.g., sodium and potassium salts), alkaline earth metal salts (e.g., calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

For the purposes of the invention Solvates refer to those forms of the compounds of the invention which form a complex in the solid or liquid state through coordination with solvent molecules. Hydrates are a specific form of solvates in which the coordination takes place with water.

For the purposes of the present invention, the substituents have, unless specified otherwise, the following meaning:

Alkyl per se and “alk” and “alkyl” in alkoxy, alkylthio, alkylamino, alkylcarbonyl, alkoxycarbonyl, alkylaminocarbonyl, alkylcarbonylamino and alkylsulfonyl represent a linear or branched alkyl radical having usually 1 to 6 (“C₁-C₆-alkyl”), preferably 1 to 4, particularly preferably 1 to 3, carbon atoms, by way of example and preferably methyl, ethyl, n-propyl, isopropyl, tert-butyl, n-pentyl and n-hexyl.

Alkoxy by way of example and preferably represents methoxy, ethoxy, n-propoxy, isopropoxy, tert-butoxy, n-pentoxy and n-hexoxy.

Alkylthio by way of example and preferably represents methylthio, ethylthio, n-propylthio, isopropylthio, tert-butylthio, n-pentylthio and n-hexylthio.

Alkylcarbonyl by way of example and preferably represents acetyl and propanoyl.

Alkylamino represents an alkylamino radical having one or two alkyl substituents (chosen independently of one another), by way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino. C₁-C₃-Alkylamino represents for example a monoalkylamino radical having 1 to 3 carbon atoms or a dialkylamino radical having 1 to 3 carbon atoms in each alkyl substituent.

Alkoxycarbonyl by way of example and preferably represents methoxycarbonyl, ethoxycarbonyl, n-propoxycarbonyl, isopropoxycarbonyl, tert-butoxycarbonyl, n-pentoxycarbonyl and n-hexoxycarbonyl.

Alkylaminocarbonyl represents an alkylaminocarbonyl radical having one or two alkyl substituents (chosen independently of one another), by way of example and preferably methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylaminocarbonyl and N-n-hexyl-N-methylaminocarbonyl. C₁-C₃-Alkylaminocarbonyl represents for example a monoalkylaminocarbonyl radical having 1 to 3 carbon atoms or a dialkylaminocarbonyl radical having 1 to 3 carbon atoms in each alkyl substituent.

Alkylcarbonylamino by way of example and preferably represents acetylamino and propanoylamino.

Alkylsulfonyl by way of example and preferably represents methylsulfonyl, ethylsulfonyl, n-propylsulfonyl, isopropylsulfonyl, tert-butylsulfonyl, n-pentylsulfonyl and n-hexylsulfonyl.

Alkylsulfoxyl by way of example and preferably represents methylsulfoxyl, ethylsulfoxyl, n-propylsulfoxyl, isopropylsulfoxyl, tert-butylsulfoxyl, n-pentylsulfoxyl and n-hexylsulfoxyl.

Cycloalkyl represents a cycloalkyl group usually having 3 to 7, preferably 5 to 6 carbon atoms, by way of example and preferably cyclopropyl, cyclobutyl, cyclopentyl, cyclohexyl and cycloheptyl.

Aryl represents a mono- or bicyclic aromatic, carbocyclic radical usually having 6 to 10 carbon atoms; by way of example and preferably phenyl and naphthyl.

Aryloxycarbonyl by way of example and preferably represents phenyloxycarbonyl and naphthyloxycarbonyl.

5- to 7-membered heterocyclyl and 5- to 7-membered heterocycle in the context of the invention represents a mono- or bicyclic, saturated or partially unsaturated heterocycle having up to three heteroatoms from the series N, O and/or S, which is linked via a ring carbon atom or a nitrogen atom of the heterocycle, and which is optionally oxo-substituted. Mention may be made by way of example and preferably of: tetrahydrofuryl, dihydrofuryl, imidazolidinyl, thiolanyl, dioxolanyl, pyrrolidinyl, pyrrolinyl, tetrahydro-2H-pyranyl, dihydropyranyl, piperidinyl, piperazinyl, morpholinyl, thiomorpholinyl, tetrahydro-2H-thiopyranyl, oxidotetrahydro-2H-thiopyranyl, 1,1-dioxidotetrahydro-2H-thiopyranyl, tetrahydrothienyl and 1,4-diazepanyl.

5- to 7-membered heteroaryl in the context of the invention generally represents an aromatic, mono- or bicyclic radical having 5 to 7 ring atoms and up to 4 heteroatoms from the series S, O and/or N. 5- to 6-membered heteroaryls having up to 4 heteroatoms are preferred. The heteroaryl radical may be linked via a carbon atom or a heteroatom. Mention may be made by way of example and preferably of: thienyl, furyl, pyrrolyl, thiazolyl, oxazolyl, imidazolyl, pyridyl, pyrimidyl, pyridazinyl, indolyl, indazolyl, benzofuranyl and benzothiophenyl.

Halogen represents fluorine, chlorine, bromine and iodine.

Preference is given in the context of the present invention to compounds of formula (I) in which

R¹ represents piperidinyl, piperazinyl, phenyl, pyridyl or thienyl,

whereby piperidinyl, piperazinyl, phenyl, pyridyl and thienyl may be substituted with 1 to 2 substituents, whereby the substituents are selected independently of one another from the group consisting of fluorine, chlorine, cyano, nitro, trifluoromethyl, trifluoromethoxy, methyl, methoxy, (C₁-C₄)-alkylamino, (C₁-C₄)-alkylcarbonyl, methylsulfonyl, (C₁-C₄)-alkoxycarbonyl and (C₁-C₄)-alkylaminothiocarbonyl,

R² represents branched (C₃-C₅)-alkyl, cyclobutyl, cyclopentyl, cyclohexyl, pyrrolidinyl, tetrahydro-2H-pyranyl, piperidinyl, tetrahydro-2H-thiopyranyl, oxidotetrahydro-2H-thiopyranyl or 1,1-dioxidotetrahydro-2H-thiopyranyl,

whereby alkyl may be substituted with 1 to 2 substituents, whereby the substituents are selected independently of one another from the group consisting of methoxy, methylthio, methylsulfonyl, methylsulfoxyl or methoxycarbonyl,

R³ represents cyclohexyl or phenyl,

whereby cyclohexyl and phenyl may be substituted with 1 to 2 substituents, whereby the substituents are selected independently of one another from the group consisting of halogen, hydroxy, (C₁-C₃)-alkyl and —OR⁶,

whereby

R⁶ represents (C₁-C₃)-alkyl,

and the salts thereof, the solvates thereof and the solvates of the salts thereof.

Preference is also given in the context of the present invention to compounds of formula (I) in which

R¹ represents piperidinyl, phenyl or pyridyl,

whereby phenyl and pyridyl may be substituted with 1 to 2 substituents, whereby the substituents are selected independently of one another from the group consisting of fluorine, chlorine, cyano, nitro, trifluoromethyl, trifluoromethoxy, methyl, methoxy and methylsulfonyl,

and

whereby piperidinyl may be substituted with 1 to 2 substituents, whereby the substituents are selected independently of one another from the group consisting of fluorine, cyano, trifluoromethyl, trifluoromethoxy, methyl, methoxy and methylsulfonyl,

R² represents branched (C₃-C₅)-alkyl, cyclobutyl, cyclopentyl, cyclohexyl, pyrrolidinyl, tetrahydro-2H-pyranyl, piperidinyl, tetrahydro-2H-thiopyranyl, oxidotetrahydro-2H-thiopyranyl or 1,1-dioxidotetrahydro-2H-thiopyranyl,

whereby alkyl may be substituted with 1 to 2 substituents, whereby the substituents are selected independently of one another from the group consisting of methoxy, methylthio, methylsulfonyl, methylsulfoxyl or methoxycarbonyl,

R³ represents cyclohexyl,

whereby cyclohexyl may be substituted with 1 to 2 substituents, whereby the substituents are selected independently of one another from the group consisting of hydroxy and methyl,

and the salts thereof, the solvates thereof and the solvates of the salts thereof.

Preference is also given in the context of the present invention to compounds of formula (I), in which

R¹ represents (C₃-C₆)-alkyl, (C₃-C₇)-cycloalkyl, 5- to 7-membered heterocyclyl, phenyl or 5- or 6-membered heteroaryl,

whereby phenyl, cycloalkyl, heterocyclyl and heteroaryl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,

R² represents (C₁-C₆)-alkyl, (C₃-C₇)-cycloalkyl, 5- to 7-membered heterocyclyl or benzyl,

whereby alkyl, cycloalkyl, heterocyclyl and benzyl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,

R³ represents (C₃-C₇)-cycloalkyl, 5- to 7-membered heterocyclyl, (C₆-C₁₀)-aryl, 5- to 7-membered heteroaryl, —CH₂—R⁴ or —CH₂—CH₂—R⁵,

whereby cycloalkyl, heterocyclyl, aryl and heteroaryl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkylthio, (C₁-C₆)-alkylcarbonyl, (C₁-C₆)-alkylsulfonyl, (C₁-C₆)-alkoxycarbonyl, (C₆-C₁₀)-aryloxycarbonyl, (C₁-C₆)-alkylaminocarbonyl, (C₁-C₆)-alkylcarbonylamino, —OR⁶ and —NR⁷R⁸,

in which alkyl may be substituted with 1 to 3 substituents, whereby the substituents are selected independently of one another from the group consisting of halogen, hydroxy, amino, hydroxycarbonyl, aminocarbonyl, (C₁-C₆)-alkoxy, (C₁-C₆)-alkylamino, (C₁-C₆)-alkylthio, phenyl, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,

in which phenyl in turn may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,

whereby

R⁶ and R⁷ represent independently of one another (C₁-C₆)-alkyl, (C₃-C₇)-cycloalkyl, 5- to 7-membered heterocyclyl, benzyl, (C₆-C₁₀)-aryl or 5- or 6-membered heteroaryl,

whereby alkyl, cycloalkyl, heterocyclyl, benzyl, aryl and heteroaryl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,

R⁸ represents hydrogen, (C₁-C₆)-alkyl or (C₃-C₇)-cycloalkyl,

R⁴ and R⁵ represent independently of one another (C₃-C₇)-cycloalkyl, 5- to 7-membered heterocyclyl, (C₆-C₁₀)-aryl or 5- to 7-membered heteroaryl,

whereby cycloalkyl, heterocyclyl, aryl and heteroaryl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,

and the salts thereof, the solvates thereof and the solvates of the salts thereof.

Preference is also given in the context of the present invention to compounds of formula (I) in which

R¹ represents tert-butyl, phenyl, thiophenyl or pyridyl,

whereby phenyl, thiophenyl and pyridyl may be substituted with 1 to 2 substituents, whereby the substituents are selected independently of one another from the group consisting of fluorine, chlorine, cyano, nitro, trifluoromethyl, trifluoromethoxy, methyl, methoxy and methylsulfonyl,

R² represents isopropyl, cyclobutyl, cyclopentyl, cyclohexyl, pyrrolidinyl, tetrahydropyranyl, piperidinyl or piperazinyl,

R³ represents cyclopentyl, cyclohexyl, phenyl, —CH₂—R⁴ or CH₂—CH₂—R⁵,

whereby cyclopentyl, cyclohexyl and phenyl may be substituted with 1 to 2 substituents, whereby the substituents are selected independently of one another from the group consisting of halogen, hydroxy, (C₁-C₆)-alkyl and —OR⁶,

whereby

R⁶ represents (C₁-C₆)-alkyl,

R⁴ and R⁵ represent independently of one another cyclopentyl, cyclohexyl or phenyl,

whereby cyclopentyl, cyclohexyl and phenyl may be substituted with 1 to 2 substituents, whereby the substituents are selected independently of one another from the group consisting of halogen, hydroxy and (C₁-C₆)-alkyl,

and the salts thereof, the solvates thereof and the solvates of the salts thereof.

Preference is also given in the context of the present invention to compounds of formula (I) in which

R¹ represents phenyl,

whereby phenyl may be substituted with 1 to 2 substituents, whereby the substituents are selected independently of one another from the group consisting of fluorine, chlorine, cyano, nitro, trifluoromethyl, trifluoromethoxy, methyl, methoxy and methylsulfonyl,

R² represents isopropyl, cyclopentyl, cyclohexyl or tetrahydropyranyl,

R³ represents cyclopentyl, cyclohexyl or phenyl,

whereby cyclopentyl, cyclohexyl and phenyl may be substituted with 1 to 2 substituents, whereby the substituents are selected independently of one another from the group consisting of halogen, hydroxy and (C₁-C₆)-alkyl,

and the salts thereof, the solvates thereof and the solvates of the salts thereof.

Preference is also given in the context of the present invention to compounds of formula (I) in which R¹ represents phenyl, whereby phenyl may be substituted with 1 to 3 substituents, whereby the substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino.

Preference is also given in the context of the present invention to compounds of formula (I) in which R¹ represents phenyl, whereby phenyl may be substituted with 1 to 2 substituents, whereby the substituents are selected independently of one another from the group consisting of fluorine, chlorine, cyano, nitro, trifluoromethyl, trifluoromethoxy, methyl, methoxy and methylsulfonyl.

Preference is also given in the context of the present invention to compounds of formula (I) in which R¹ represents phenyl, whereby phenyl may be substituted with 1 to 2 fluorine substituents.

Preference is also given in the context of the present invention to compounds of formula (I) in which R¹ represents 4-fluorophenyl.

Preference is also given in the context of the present invention to compounds of formula (I) in which R¹ represents phenyl.

Preference is also given in the context of the present invention to compounds of formula (I) in which R² represents (C₃-C₇)-cycloalkyl.

Preference is also given in the context of the present invention to compounds of formula (I) in which R² represents cyclobutyl, cyclopentyl or cyclohexyl.

Preference is also given in the context of the present invention to compounds of formula (I) in which R² represents 5- to 7-membered heterocyclyl.

Preference is also given in the context of the present invention to compounds of formula (I) in which R² represents pyrrolidinyl, tetrahydropyranyl, piperidinyl or piperazinyl.

Preference is also given in the context of the present invention to compounds of formula (I) in which R² represents pyrrolidinyl, tetrahydro-2H-pyranyl, piperidinyl, tetrahydro-2H-thiopyranyl, oxidotetrahydro-2H-thiopyranyl or 1,1-dioxidotetrahydro-2H-thiopyranyl.

Preference is also given in the context of the present invention to compounds of formula (I) in which R² represents (C₁-C₆)-alkyl, whereby alkyl may be substituted with 1 to 3 substituents, whereby the substituents are selected independently of one another from the group consisting of hydroxy, amino, hydroxycarbonyl, aminocarbonyl, (C₁-C₆)-alkoxy, (C₁-C₆)-alkylamino, (C₁-C₆)-alkoxycarbonyl, (C₁-C₆)-alkylaminocarbonyl and (C₁-C₆)-alkylcarbonylamino.

Preference is also given in the context of the present invention to compounds of formula (I) in which R² represents (C₁-C₆)-alkyl, whereby alkyl may be substituted with 1 to 3 substituents, whereby the substituents are selected independently of one another from the group consisting of hydroxy, amino, hydroxycarbonyl, aminocarbonyl, (C₁-C₆)-alkoxy, (C₁-C₆)-alkylamino, (C₁-C₆)-alkylthio, (C₁-C₆)-alkoxycarbonyl, (C₁-C₆)-alkylaminocarbonyl, (C₁-C₆)-alkylsulfonyl, (C₁-C₆)-alkylsulfoxyl and (C₁-C₆)-alkylcarbonylamino.

Preference is also given in the context of the present invention to compounds of formula (I) in which R² represents branched (C₃-C₅)-alkyl, whereby alkyl may be substituted with 1 to 2 substituents, whereby the substituents are selected independently of one another from the group consisting of methoxy, methylthio, methylsulfonyl, methylsulfoxyl or methoxycarbonyl.

Preference is also given in the context of the present invention to compounds of formula (I) in which R² represents 1-methoxypropan-2-yl, 1-methylsulfonylpropan-2-yl or 1-methylsulfoxylpropan-2-yl.

Preference is also given in the context of the present invention to compounds of formula (I) in which R² represents isopropyl.

Preference is also given in the context of the present invention to compounds of formula (I) in which R³ represents cyclohexyl, whereby cyclohexyl may be substituted with 1 to 2 substituents, whereby the substituents are selected independently of one another from the group consisting of hydroxy and methyl.

Preference is also given in the context of the present invention to compounds of formula (I) in which R³ represents 2-hydroxy-4-methylcyclohexyl.

Preference is also given in the context of the present invention to compounds of formula (I) in which R³ represents 2-hydroxy-4-methylcyclohexyl, whereby the methyl group and the linkage of the cyclohexyl to the carbonyl group are in positions trans to one another.

Preference is also given in the context of the present invention to compounds of formula (I) in which R³ represents trans-4-methylcyclohexyl.

Preference is also given in the context of the present invention to compounds of formula (I) in which R³ represents phenyl, whereby phenyl may be substituted with 1 to 2 substituents, whereby the substituents are selected independently of one another from the group consisting of halogen and (C₁-C₆)-alkyl.

Preference is also given in the context of the present invention to compounds of formula (I) in which R³ represents phenyl, whereby phenyl may be substituted with 1 to 2 substituents, whereby the substituents are selected independently of one another from the group consisting of chlorine and methyl.

The invention further relates to a process for preparing the compounds of formula (I), whereby compounds of formula

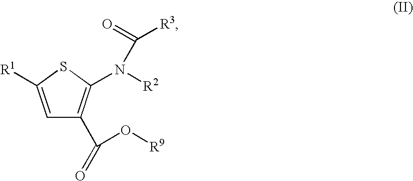

in which

R¹, R² and R³ have the abovementioned meaning, and

R⁹ represents alkyl, preferably methyl, ethyl or tert-butyl,

are reacted with bases or acids.

In the case where R⁹ represents methyl or ethyl, the reaction is generally effected with a base in inert solvents, preferably in a temperature range from room temperature to reflux of the solvents under atmospheric pressure.

Examples of bases are alkali metal hydroxides such as sodium, lithium or potassium hydroxide, or alkali metal carbonates such as cesium carbonate, sodium or potassium carbonate, optionally in aqueous solution, with lithium hydroxide in water being preferred.

Examples of inert solvents are ethers such as 1,2-dimethoxyethane, dioxane, tetrahydrofuran, glycol dimethyl ether or diethylene glycol dimethyl ether, or alcohols such as methanol, ethanol, n-propanol, isopropanol, n-butanol or tert-butanol, or mixtures of solvents, with dioxane or tetrahydrofuran being preferred.

In the case where R⁹ represents tert-butyl, the reaction is generally effected with an acid in inert solvents, preferably in a temperature range from room temperature to reflux of the solvents under atmospheric pressure.

Reactions with trifluoroacetic acid in methylene chloride, with hydrogen chloride in dioxane or with hydrochloric acid in dioxane are preferred.

Nitrogen atoms in heterocycles of the radicals R¹, R² and R³ of the compounds of formula (II) can optionally be further substituted before the hydrolysis to give compounds of formula (I), as described for example in the experimental section.

The compounds of formula (II) are known or can be prepared by reacting compounds of formula

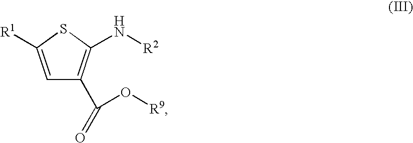

in which

R¹, R² and R⁹ have the abovementioned meaning,
with compounds of formula

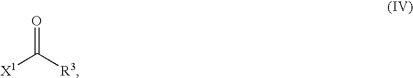

in which

R³ has the abovementioned meaning, and

X¹ represents halogen, preferably chlorine or bromine.

The reaction is effected in inert solvents or in situ with the acylating reagent, optionally in the presence of a base, preferably in a temperature range from 0° C. to 120° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, 1,2-dimethoxyethane, glycol dimethyl ether or diethylene glycol dimethyl ether, hydrocarbons such as benzene, xylene, toluene, hexane, cyclohexane or petroleum fractions, or carboxamides such as N,N-dimethylformamide or N,N-dimethylacetamide, alkyl nitriles such as acetonitrile, or heteroaromatic compounds such as pyridine, or ethyl acetate, with pyridine or acetonitrile being preferred.

Examples of bases are alkali metal carbonates such as cesium carbonate, sodium or potassium carbonate, alkali metal acetates such as sodium acetate or other bases such as triethylamine, diisopropylethylamine or pyridine, preferably diisopropylethylamine, triethylamine or pyridine.

If the radicals R³ comprise functional groups such as, for example, hydroxy or amino, these groups are provided with suitable protective groups known from the literature, such as, for example, acetyl, benzyl, benzyloxycarbonyl or tert-butyloxycarbonyl, which are removed after the reaction to give the compounds of formula (I) (Lit: P. J. Kocienski: “Protecting Groups”, Georg Thieme Verlag, Stuttgart, N.Y., 1994, ISBN 3-13-135601-4).

The compounds of formula (IV) are known or can be synthesized from the appropriate precursors by known processes.

The compounds of formula (III) are known or can be prepared by reacting compounds of formula

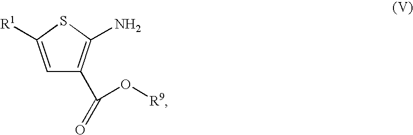

in which

R¹ and R⁹ have the abovementioned meaning,

with aldehydes, ketones, orthoesters or enol ethers which comprise the radical R²,

under the conditions of a reductive amination.

The reaction is generally effected in inert solvents in the presence of a reducing agent and acetic acid, preferably in a temperature range from room temperature to reflux of the solvent under atmospheric pressure.

Examples of inert solvents are halohydrocarbons such as methylene chloride or 1,2-dichloroethane, ethers such as dioxane or tetrahydrofuran, alcohols such as methanol or ethanol, or N,N-dimethylformamide, with methylene chloride or 1,2-dichloroethane being preferred.

Examples of reducing agents are sodium borohydride, sodium cyanoborohydride, sodium triacetoxyborohydride or tetrabutylammonium borohydride, with sodium triacetoxyborohydride being preferred.

The compounds of formula (V) are known or can be synthesized from the appropriate precursors by known processes (K. Gewald et al., Chem. Ber. 1966, 94-100).

The reaction is optionally effected in the presence of Lewis acids such as, for example, titanium tetrachloride (analogous to J. F. Parlow, M. S. South, Tetrahedron 2003, 59, 7695-7701).

Aldehydes, ketones, orthoesters and enol ethers which comprise the radical R² are known or can be synthesized from the appropriate precursors by known processes.

In an alternative process, the compounds of formula (II) can be prepared by reacting compounds of formula

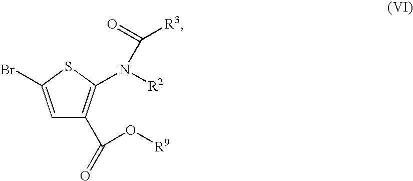

in which

R², R³ and R⁹ have the abovementioned meaning,

with compounds of formula

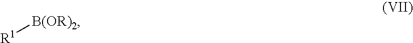

in which

R¹ has the abovementioned meaning, and

R represents hydrogen or (C₁-C₄)-alkyl.

The reaction is generally effected under Suzuki reaction conditions in inert solvents in the presence of a catalyst, optionally in the presence of an additional reagent, preferably in a temperature range from room temperature to 130° C. under atmospheric pressure (S. Kotha, K. Lahiri, D. Kashinath, Tetrahedron 2002, 58 (48), 9633-9695 and N. Miyaura, A. Suzuki, Chem. Rev. 1995, 95, 2457-2483).

Examples of catalysts are palladium catalysts usually used for Suzuki reaction conditions, with catalysts such as, for example, dichlorobis(triphenylphosphine)palladium, tetrakistriphenylphosphinepalladium(0), palladium(II) acetate, 1,1′-bis[(diphenylphosphino)ferrocene]palladium(II) chloride (1:1) complex with dichloromethane being preferred.

Examples of additional reagents are potassium acetate, cesium, potassium or sodium carbonate, barium hydroxide, potassium tert-butoxide, cesium fluoride or potassium phosphate carried out, with additional reagents such as, for example, potassium acetate and/or aqueous sodium carbonate solution being preferred.

Examples of inert solvents are ethers such as dioxane, tetrahydrofuran or 1,2-dimethoxyethane, hydrocarbons such as benzene, xylene or toluene, or other solvents such as nitrobenzene, dimethylformamide, dimethylacetamide, dimethylsulfoxide or N-methylpyrrolidone, with solvents such as, for example, dimethylformamide, dimethylacetamide, dimethylsulfoxide or 1,2-dimethoxyethane being preferred.

The compounds of formula (VII) are known or can be synthesized from the appropriate precursors by known processes.

The compounds of formula (VI) are known or can be prepared by reacting compounds of formula

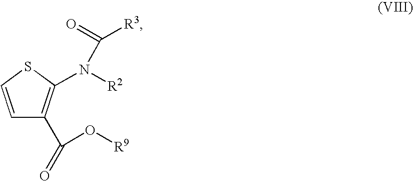

in which

R², R³ and R⁹ have the abovementioned meaning,

with N-bromosuccinimide in halohydrocarbons such as dichloromethane, chloroform or tetrachloromethane and mixtures thereof, with a mixture of chloroform and tetrachloromethane being preferred.

The compounds of formula (VIII) are known or can be prepared by reacting compounds of formula

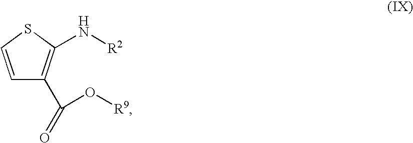

in which

R² and R⁹ have the abovementioned meaning,

with compounds of formula (IV) under the reaction conditions indicated for reacting compounds of formula (III) with compounds of formula (IV).

The compounds of formula (IX) are known or can be synthesized from the appropriate precursors by known processes or can be prepared in analogy to the compounds of formula (III).

In an alternative process, the compounds of formula (II) can be prepared by reacting compounds of formula

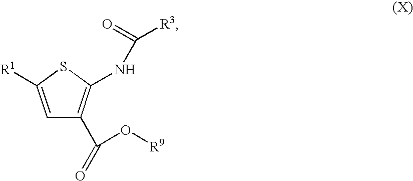

in which

R¹, R³ and R⁹ have the abovementioned meaning, with compounds of formula
R²—X² (XI),

in which

R² has the abovementioned meaning, and

X² represents halogen, preferably iodine or bromine, or a sulfonic ester.

The reaction is generally effected in inert solvents in the presence of a base, preferably in a temperature range from room temperature to reflux of the solvent under atmospheric pressure.

Examples of bases are alkali metal carbonates such as cesium carbonate, sodium or potassium carbonate, or sodium or potassium methanolate, or sodium or potassium ethanolate or potassium tert-butoxide, or amides such as sodium amide, lithium bis(trimethylsilyl)amide or lithiumdiisopropylamide, or organometallic compounds such as butyllithium or phenyllithium, or other bases such as sodium hydride, DBU, preferably potassium tert-butoxide, cesium carbonate, DBU, sodium hydride, potassium carbonate or sodium carbonate.

Examples of inert solvents are halohydrocarbons such as methylene chloride, trichloromethane or 1,2-dichloroethane, ethers such as dioxane, tetrahydrofuran or 1,2-dimethoxyethane, or other solvents such as acetone, dimethylformamide, dimethylacetamide, 2-butanone or acetonitrile, preferably tetrahydrofuran, methylene chloride, acetone, 2-butanone, acetonitrile, dimethylformamide or 1,2-dimethoxyethane.

The compounds of formula (XI) are known or can be synthesized from the appropriate precursors by known processes.

The compounds of formula (X) are known or can be prepared by reacting compounds of formula (V) with compounds of formula (IV) under the reaction conditions indicated for reacting compounds of formula (III) with compounds of formula (IV).

Preparation of the compounds of the invention can be illustrated by the following synthesis schemes.

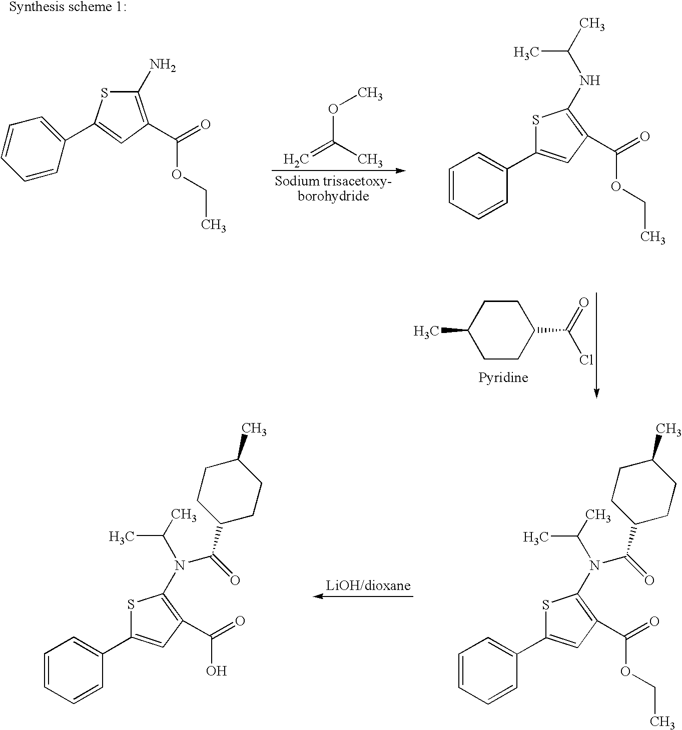

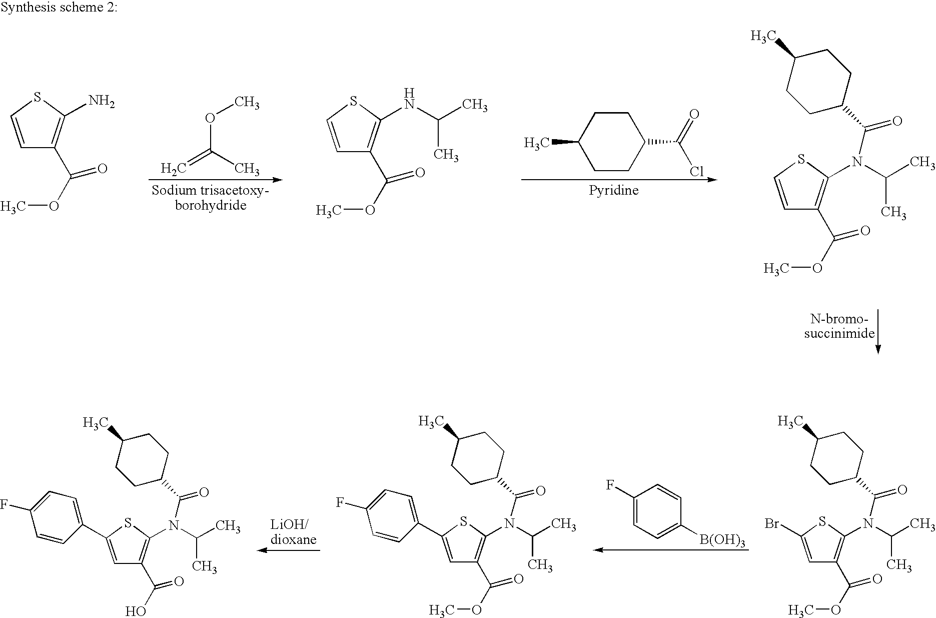

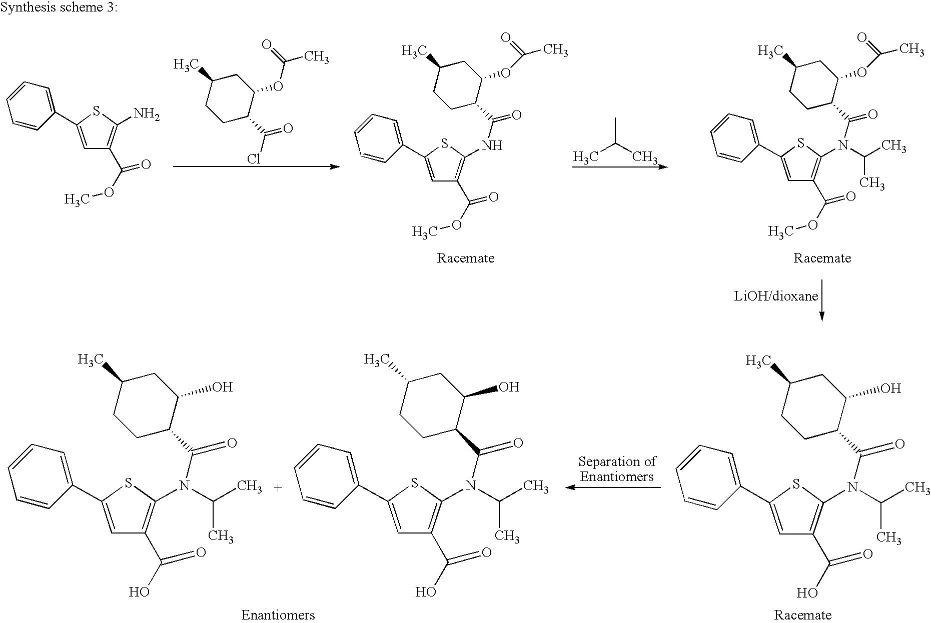

The compounds of the invention show a valuable range of effects which could not have been predicted. They show an antiviral effect on representatives of the Flaviviridae family, especially on the hepatitis C virus.

Another aspect of the present invention is the use of the compounds of the invention for the treatment and/or prophylaxis of diseases, especially of infections with viruses, in particular the aforementioned viruses, and of the infectious diseases caused thereby. A viral infection means hereinafter both an infection with a virus and a disease caused by an infection with a virus.

Examples of indications which may be mentioned are:

1. treatment of acute and chronic hepatitis C infections and the prevention, alleviation or elimination of concomitant phenomena and sequelae such as, for example, hepatic fibrosis, cirrhosis of the liver, liver cancer (hepatocellular carcinoma) and/or the reduction of the number of viral genome copies in a patient;

2. treatment and prophylaxis of patients undergoing organ transplants, especially in cases of a hepatitis C infection of the organ donor or organ recipient;

3. treatment of HCV infections in AIDS patients and patients who are infected with HIV (human immunodeficiency virus) (co-infection of HIV with hepatitis C leads to a rapid deterioration in the clinical condition);

4. treatment of HCV infections in patients who are infected with HBV (hepatitis B virus) or other hepatotrophic viruses (for example hepatitis A virus, hepatitis G virus);

5. treatment of diseases with viruses related to HCV, such as, for example, yellow fever virus, dengue virus, West Nile virus, spring-summer encephalitis virus, Japanese encephalitis virus;

6. treatment of mammals with related animal viruses such as, for example, pestiviruses;

7. treatment of materials or biological agents in order to prevent transmission of hepatitis C or reduce such a risk (e.g., for blood and blood products, blood donation utensils or surgical instruments).

The present invention further relates to the use of the compounds of the invention for the production of a medicament for the treatment and/or prophylaxis of diseases, especially of the aforementioned diseases.

The compounds of the invention are preferably used for the production of medicaments which are suitable for the prophylaxis and/or treatment of viral infections with hepatitis C virus or other members of the Flaviviridae family.

The present invention further relates to the use of the compounds of the invention alone or in combination with other active ingredients for the treatment and/or prophylaxis of diseases, especially the aforementioned diseases.

The present invention further relates to medicaments which comprise at least one compound of the invention, preferably together with interferon (pegylated or non-pegylated) or with ribavirin or with one or more anti-HCV agents or with a combination thereof, and the use thereof for the aforementioned purposes.

The present invention further relates to a method for the treatment and/or prophylaxis of diseases, especially the aforementioned diseases, using an antivirally effective amount of the compounds of the invention.

Preference is given in the context of the present invention to a method for the treatment of an HCV infection by administering an effective amount of at least one of the compounds of the invention, of a pharmacologically acceptable salt, solvate or solvate of a salt thereof or of a medicament as described above, alone or together with interferon (pegylated or non-pegylated) or with ribavirin or with one or more anti-HCV agents or with a combination thereof, which can be administered together or separately.

Preference is also given in the context of the present invention to a method for the prophylaxis of an HCV infection by administering an effective amount of at least one of the compounds of the invention, of a pharmacologically acceptable salt, solvate or solvate of a salt thereof or of a medicament as described above, alone or together with interferon (pegylated or non-pegylated) or with ribavirin or with one or more anti-HCV agents or with a combination thereof, which can be administered together or separately.

Medicaments of the present invention may comprise one or more additional active agents, preferably selected from the group of antiviral agents, immunomodulatory agents, HCV protease inhibitors, HCV polymerase inhibitors, inhibitors of another target in the HCV lifecycle, HIV inhibitors, HAV inhibitors and HBV inhibitors. Examples of such agents are listed and explained below.

Preferred examples of some of these agents are ribavirin and amantadine (antiviral agents), class I interferons, class II interferons and pegylated interferons (immunomodulatory agents), inhibitors of HCV NS5B polymerase, HCV NS3 helicase, HCV protease or IRES (inhibitors of another target in the HCV lifecycle), nucleoside inhibitors, non-nucleoside inhibitors, protease inhibitors, fusion inhibitors and integrase inhibitors of HIV (HIV inhibitors) or agents which inhibit HBV DNA polymerase, or hepatitis B vaccines (HBV inhibitors).

The present invention thus also encompasses a combination therapy in which at least one of the compounds of the invention or a pharmacologically acceptable salt, solvate or solvate of a salt thereof is administered together with at least one additional agent selected from the group of antiviral agents, immunomodulatory agents, HCV protease inhibitors, HCV polymerase inhibitors, inhibitors of another target in the HCV lifecycle, HIV inhibitors, HAV inhibitors and HBV inhibitors. The additional agents may be combined with the compounds of the invention to give a single pharmaceutical dosage form. Alternatively, these additional agents can be administered separately. Such additional agents can be administered before, during or after the administration of a compound of the invention or of a pharmacologically acceptable salt, solvate or solvate of a salt thereof.

Definitions:

The term “anti-viral agent” means an agent which inhibits the formation and/or replication of a virus. This includes agents which intervene in mechanisms of the host or of the virus which are necessary for the formation and/or replication of a virus. Examples of antiviral agents are ribavirin, amantadine, VX-497 (merimepodib, Vertex Pharmaceuticals), levovirin, viramidine, Ceplene (Maxamine), XTL-001 and XTL-002 (XTL-Biopharmaceuticals).

The term “anti-HCV agent” means an agent which diminishes or prevents hepatitis C-related disease symptoms. Such an agent may be an antiviral agent, an immunomodulatory agent, an HCV protease inhibitor, an HCV polymerase inhibitor or an inhibitor of another target in the HCV lifecycle.

The term “immunomodulatory agent” means an agent which strengthens the immune response or controls harmful immune reactions. Examples of immunomodulatory agents are class I interferons (such as alpha-, beta-, delta- and omega-interferons, tau-interferons, consensus interferons and asialo-interferons), class II interferons (such as gamma-interferons) and pegylated interferons.

The term “HCV protease inhibitor” means an agent which inhibits the function of the HCV NS2/3 metalloprotease or the NS3/4A serine protease. Examples of HCV NS3/4A serine protease inhibitors are BILN 2061 (Boehringer Ingelheim), or VX-950/LY-570310 (Vertex/Eli Lilly).

The term “HCV polymerase inhibitor” means an agent which inhibits the function of HCV polymerase. This includes for example inhibitors of the HCV NS5B polymerase. HCV polymerase inhibitors include non-nucleosides, for example compounds which are described in WO 02/100846 and WO 02/100851 (Shire), WO 01/85172 and WO 02/098424 (GSK), WO 00/06529 and WO 02/06246 (Merck), WO 01/47883 and WO 03/000254 (Japan Tobacco) and EP 1 256 628 (Agouron). HCV polymerase inhibitors also include nucleoside analogues, for example compounds which are described in WO 01/90121 (Idenix), WO 02/069903 (Biochryst Pharmaceuticals), WO 02/057287 and WO 02/057425 (Merck/Isis). Further examples of HCV polymerase inhibitors are JTK-002, JTK-003 and JTK-109 (Japan Tobacco).

The term “inhibitor of another target in the HCV lifecycle” means an agent which inhibits the formation and/or replication of HCV in a way other than by inhibiting the function of an HCV protease or of HCV polymerase. This includes agents which intervene in mechanisms of the host or of HCV which are necessary for the formation and/or replication of HCV. Inhibitors of another target in the HCV lifecycle include agents which inhibit for example a helicase or an IRES as target. A specific example of an inhibitor of another target in the HCV lifecycle is ISIS-14803 (ISIS-Pharmaceuticals).

The term “HIV inhibitor” means an agent which inhibits the formation and/or replication of HIV. This includes agents which intervene in mechanisms of the host or of HIV which are necessary for the formation and/or replication of HIV. HIV inhibitors include for example nucleoside inhibitors, non-nucleoside inhibitors, protease inhibitors, fusion inhibitors and integrase inhibitors.

The term “HAV inhibitor” means an agent which inhibits the formation and/or replication of HAV. This includes agents which intervene in mechanisms of the host or of HAV which are necessary for the formation and/or replication of HAV. HAV inhibitors include for example hepatitis A vaccines [e.g., Havrix® (GSK), VAQTA® (Merck), Avaxim® (Aventis Pasteur)].

The term “HBV inhibitor” means an agent which inhibits the formation and/or replication of HBV. This includes agents which intervene in mechanisms of the host or of HBV which are necessary for the formation and/or replication of HBV. HBV inhibitors include for example agents which inhibit HBV DNA polymerase, or hepatitis B vaccines. Specific examples of HBV inhibitors are: lamivudine (Epivir-HBV®, adefovir dipivoxil, entecavir, FTC (Coviracil®), DAPD (DXG), L-FMAU (Clevudine®), AM365 (Amrad), Ldt (Telbivudine), monoval-LdC (Valtorcitabine), BAY 41-4109 (Bayer), ACH-126,443 (L-Fd4C) (Achillion),

MCC₄78 (Eli Lilly), racivir (RCV), fluoro-L- and D-nucleosides, robustaflavones, ICN2001-3 (ICN), Bam 205 (Novelos), XTL-001 (XTL), imino sugars (Nony-DNJ) (Synergy), HepBzyme, and immunomodulatory products such as, for example, interferon alpha-2b, HE2000 (Hollis-Eden), theradigm (Epimmune), EHT899 (Enzo Biochem), thymosin alpha-1 (Zadaxin®), HBV DNA vaccine (PowderJect), HBV DNA vaccine (Jefferon Center), HBV antigen (OraGen), BayHep B® (Bayer), Nabi-HB® (Nabi) and anti-hepatitis B (Cangene), and HBV vaccines such as, for example Engerix B, Recombivax HB, GenHevac B, Hepacare, Bio-Hep B, TwinRix, Comvax, Hexavac.

The term “class I interferons” means an interferon selected from a group of interferons which all bind to the type I receptor. This includes natural and synthetically prepared class I interferons. Examples of class I interferons are alpha-, beta- and omega-interferons, tau-interferons, consensus-interferons and asialo-interferons.

The term “class II interferons” means an interferon selected from a group of interferons which all bind to the type II receptor. Examples of class II interferons are gamma-interferons.

The term “treatment” means the administration of a medicament according to the present invention in order to alleviate or eliminate the symptoms of hepatitis C disease and/or reduce the amount of virus.

The term “prophylaxis” means the administration of a medicament according to the present invention after an infection with HCV but before the appearance of symptoms of a disease and/or before detection of HCV in the blood.

The compounds of the invention can act systemically and/or locally. For this purpose, they can be administered in a suitable way such as, for example, orally, parenterally, pulmonally, nasally, sublingually, lingually, buccally, rectally, dermally, transdermally, conjunctivally, otically or as an implant or stent.

The compounds of the invention can be administered in administration forms suitable for these administration routes.

Suitable for oral administration are administration forms which function according to prior art and deliver the compounds of the invention rapidly and/or in modified fashion, and which contain the compounds of the invention in crystalline and/or amorphized and/or dissolved form, such as, for example, tablets (uncoated or coated tablets, for example having enteric coatings or coatings which dissolve with a delay or are insoluble and control the release of the compound of the invention), tablets or films/wafers which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g., intravenous, intraarterial, intracardiac, intraspinal or intralumbar) or with inclusion of an absorption (e.g., intramuscular, subcutaneous, intracutaneous, percutaneous or intraperitoneal). Administration forms suitable for parenteral administration are, inter alia, preparations for injections and infusions in the form of solutions, suspensions, emulsions, lyophilizates or sterile powders.

Suitable for the other administration routes are, for example, pharmaceutical forms for inhalation (inter alia powder inhalers, nebulizers), nasal drops, solutions, sprays; tablets films/wafers or capsules, for lingual, sublingual or buccal administration, suppositories, preparations for the ears or eyes, vaginal capsules, aqueous suspensions (lotions, shaking mixtures), lipophilic suspensions, ointments, creams, transdermal therapeutic systems, milk, pastes, foams, dusting powders, implants or stents.

The compounds of the invention can be converted into the stated administration forms. This can take place in a manner known per se by mixing with inert, nontoxic, pharmaceutically suitable excipients. These excipients include, inter alia, carriers (for example microcrystalline cellulose, lactose, mannitol), solvents (e.g., liquid polyethylene glycols), emulsifiers and dispersing or wetting agents (for example sodium dodecyl sulfate, polyoxysorbitan oleate), binders (for example polyvinylpyrrolidone), synthetic and natural polymers (for example albumin), stabilizers (e.g., antioxidants such as, for example, ascorbic acid), colours (e.g., inorganic pigments such as, for example, iron oxides) and taste and/or odor corrigents.

The present invention further relates to medicaments which comprise at least one compound of the invention, usually together with one or more inert, nontoxic, pharmaceutically suitable excipients, and to the use thereof for the aforementioned purposes.

It has generally proven advantageous to administer on intravenous administration amounts of about 0.001 to 20 mg/kg, preferably about 0.1 to 5 mg/kg, of bodyweight to achieve effective results, and on oral administration the dosage is about 0.01 to 50 mg/kg, preferably 0.5 to 10 mg/kg, of bodyweight.

It may nevertheless be necessary where appropriate to deviate from the stated amounts, in particular as a function of the bodyweight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of an administration of larger amounts be advisable to divide these into a plurality of individual doses over the day.

The percentage data in the following tests and examples are, unless indicated otherwise, percentages by weight; parts are parts by weight. Solvent ratios, dilution ratios and concentration data for the liquid/liquid solutions are in each case based on volume.

### DETAILED DESCRIPTION OF PREFERRED EMBODIMENTS

### A. Examples

Abbreviations Used:

| | |
|---|---|
| BINAP | 2,2′-Bis(diphenylphosphino)-1,1′-binaphthyl |
| CDCl3 | Deuterochloroform |
| CD3CN | Deuteroacetonitrile |
| conc. | Concentrated |
| DCI | Direct chemical ionization (in MS) |
| DCM | Dichloromethane |
| DIEA | N,N-Diisopropylethylamine (Hünig's base) |
| DMAP | 4-N,N-Dimethylaminopyridine |
| DMSO | Dimethylsulfoxide |
| DMF | N,N-Dimethylformamide |
| EA | Ethyl acetate (acetic acid ethyl ester) |
| EI | Electron impact ionization (in MS) |
| ESI | Electrospray ionization (in MS) |
| H | Hour |
| HPLC | High pressure, high performance liquid chromatography |
| LC-MS | Coupled liquid chromatography-mass spectroscopy |
| LDA | Lithium diisopropylamide |
| LiOH | Lithium hydroxide |
| m.p. | Melting point |
| MS | Mass spectroscopy |
| NMR | Nuclear magnetic resonance spectroscopy |
| RP-HPLC | Reverse Phase HPLC |
| RT | Room temperature |
| Rₜ | Retention time (in HPLC) |
| sat. | Saturated |
| THF | Tetrahydrofuran |
| TLC | Thin-layer chromatography |

General LCMS and HPLC Methods:

Method 1 (HPLC): Instrument: HP 1100 with DAD detection; column: Kromasil RP-18, 60 mm×2 mm, 3.5 μm; eluent A: 5 ml of HClO₄/l of water, eluent B: acetonitrile; gradient: 0 min 2% B, 0.5 min 2% B, 4.5 min 90% B, 6.5 min 90% B; flow rate: 0.75 ml/min; oven: 30° C.; UV detection: 210 nm.

Method 2 (HPLC, preparative separation): Column: CromSil C₁8, 250 mm×30 mm; eluent A: water, eluent B: acetonitrile; gradient: 3 min 10% B→31 min 90% B→34 min 90% B→34.01 min 10% B; running time: 38 min; flow rate: 50 ml/min; UV detection: 210 nm.

Method 3 (LC-MS): Instrument: Micromass Quattro LCZ with HPLC Agilent series 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 4 (LC-MS): MS instrument type: Micromass ZQ; HPLC instrument type: HP 1100 series; UV DAD; column: Phenomenex Synergi 2 g Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.

Method 5 (LC-MS): MS instrument type: Micromass ZQ; HPLC instrument type: Waters Alliance 2795; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 6 (HPLC): Instrument: HP 1100 with DAD detection; column: Kromasil RP-18, 60 mm×2 mm, 3.5 μm; eluent A: 5 ml of HClO_{4/1} of water, eluent B: acetonitrile; gradient: 0 min 2% B, 0.5 min 2% B, 4.5 min 90% B, 9 min 90% B; flow rate: 0.75 ml/min; oven: 30° C.; UV detection: 210 nm.

Starting Compounds

### Example 1A

### Ethyl 2-amino-5-phenylthiophene-3-carboxylate

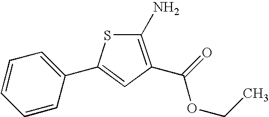

94.1 g (832 mmol) of ethyl cyanoacetate and 26.7 g (832 mmol) of sulfur are introduced into 200 ml of DMF under argon and, at RT, 45.2 g (446 mmol) of triethylamine are added. 80.0 g (666 mmol) of phenylacetaldehyde are added dropwise, and the mixture is stirred at RT for 3 h. It is poured into water and vigorously stirred for 15 min, and the precipitate is filtered off with suction. Purification by recrystallization from ethanol results in 74.0 g (45% of theory) of product.

LC-MS (Method 3): Rₜ=2.64 min

MS (ESIpos): m/z=248 (M+H)⁺.

¹H-NMR (300 MHz, DMSO-d₆): δ=7.48-7.42 (m, 4H), 7.37-7.29 (m, 2H), 7.23 (s, 1H), 7.19 (tt, 1H), 4.21 (q, 2H), 1.28 (t, 3H).

### Example 2A

### Ethyl 2-isopropylamino-5-phenylthiophene-3-carboxylate

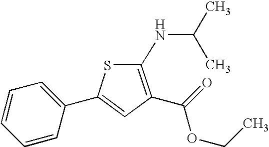

30.0 g (121 mmol) of ethyl 2-amino-5-phenylthiophene-3-carboxylate are introduced into 560 ml of 1,2-dichloroethane under argon and, at RT, 35.0 g (485 mmol) of 2-methoxypropene are added. The mixture is stirred at RT for 1 h. Then 29.1 g (485 mmol) of glacial acetic acid and 51.4 g (243 mmol) of sodium triacetoxyborohydride are added, and the mixture is stirred at RT for 2 h. After the addition of a saturated sodium bicarbonate solution and the separation of the phases the aqueous phase is extracted three times with ethyl acetate. The combined organic phases are washed with a saturated sodium chloride solution, dried over sodium sulfate, filtered and concentrated. The 35.0 g (99% of theory) of product can be reacted on without further purification.

LC-MS (Method 4): Rₜ=3.29 min

MS (ESIpos): m/z=290 (M+H)⁺.

¹H-NMR (300 MHz, DMSO-d₆): δ=7.54-7.46 (m, 3H), 7.38-7.30 (m, 3H), 7.20 (tt, 1H), 4.22 (q, 2H), 3.61-3.48 (m, 1H), 1.30 (d, 6H), 1.29 (t, 3H).

General Procedure [A]: Reductive Amination of 2-aminothiophenes with Aldehydes and Ketones

Reaction with Cyclic Ketones without Dehydrating Reagents

8.1 mmol (1.0 equivalent) of the 2-aminothiophene are introduced into 50 ml of dichloroethane under argon and, at room temperature, 32.3 mmol (4.0 equivalents) of the carbonyl compound are added. The mixture is stirred at this temperature for 2 h, then 32.3 mmol (4.0 equivalents) of acetic acid and 16.2 mmol (2.0 equivalents) of sodium triacetoxyborohydride are added, and the mixture is stirred at 40° C. for 16 h. After cooling, a saturated sodium bicarbonate solution is cautiously added, the phases are separated, and the aqueous phase is extracted three times with dichloromethane. The combined organic phases are washed once with a saturated sodium chloride solution, dried over sodium sulfate and filtered, and the solvent is removed in vacuo. The product is then purified by chromatography on silica gel with cyclohexane/ethyl acetate mixtures.

Alternatively, the mixture can be worked up by adding 2 ml of 1N hydrochloride acid, and the precipitate which forms is filtered off, washed with methanol and dried in vacuo. The product is, optionally, subsequently purified by chromatography on silica gel with cyclohexane/ethyl acetate mixtures.

### Example 3A

### Ethyl 2-(cyclopentylamino)-5-phenylthiophene-3-carboxylate

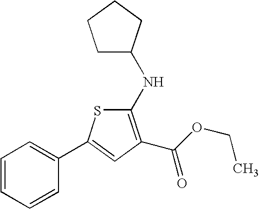

Starting with 2.00 g (8.09 mmol) of 2-aminothiophene from Example 1A and 2.72 g of cyclopentanone, general procedure [A] results after chromatography in 992 mg (32% of theory) of product.

HPLC (Method 1): Rₜ=6.03 min

MS (CI-pos): m/z=316 (M+H)⁺

### Example 4A

### Ethyl 2-(cyclohexylamino)-5-phenylthiophene-3-carboxylate

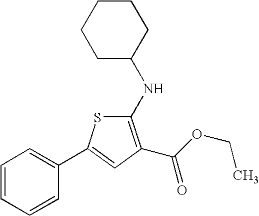

Starting with 2.00 g (8.09 mmol) of 2-aminothiophene from Example 1A and 3.18 g of cyclohexanone, general procedure [A] results after chromatography in 1.44 mg (52% of theory) of product.

HPLC (Method 1): Rₜ4=6.23 min

MS (CI-pos): m/z=330 (M+H)⁺

### Example 5A

### Ethyl 2-(cyclobutylamino)-5-phenylthiophene-3-carboxylate

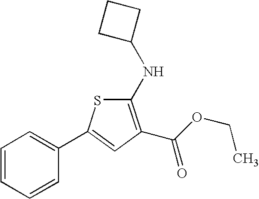

Starting with 2.0 g (8.1 mmol) of 2-aminothiophene from Example 1A and 2.3 g (32.3 mmol) of cyclobutanone, general procedure [A] results after chromatography in 1.17 mg (48% of theory) of product.

HPLC (Method 1): Rₜ=6.01 min

MS (ESI-pos): m/z=302 (M+H)⁺

### Example 6A

### Ethyl 2-[isopropyl(trans-4-methylcyclohexanecarbonyl)amino]-5-phenylthiophene-3-carboxylate

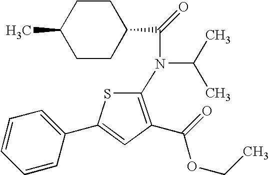

10.0 g (70.3 mmol) of trans-4-methylcyclohexanecarboxylic acid are introduced into 200 ml of dichloromethane under argon, and 18.0 g (142 mmol) of oxalyl chloride and one drop of DMF are added. The mixture is stirred at RT overnight. After concentrating the solution, the residue is added under argon to a solution of 6.00 g (20.7 mmol) of ethyl 2-isopropylamino-5-phenylthiophene-3-carboxylate and a spatula tip of DMAP in 50 ml of pyridine. The mixture is heated at 120° C. overnight. It is then concentrated, water is added, the phases are separated, the aqueous phase is extracted twice with dichloromethane, and the combined organic phases are dried over sodium sulfate, filtered and concentrated. Column chromatography on silica 60 (mobile phase: toluene) results in 5.00 g (58% of theory) of product. Alternatively purification is also possible by preparative HPLC (RP-18 column, mobile phase: acetonitrile-water gradient 95:5→5:95).

LC-MS (Method 5): Rₜ=3.29 min

MS (ESIpos): m/z=414 (M+H)⁺.

¹H-NMR (300 MHz, DMSO-d₆): δ=7.82-7.71 (m, 3H), 7.51-7.35 (m, 3H), 4.79 (sept, 1H), 4.31-4.16 (m, 2H), 2.20-2.05 (m, 1H), 1.75-1.43 (m, 5H), 1.38-1.26 (m, 2H), 1.25 (t, 3H), 1.16 (d, 3H), 0.90 (d, 3H), 0.75 (d, 3H), 0.74-0.52 (m, 2H).

### Example 7A

### Ethyl 2-[(2,4-dichlorobenzoyl)isopropylamino]-5-phenylthiophene-3-carboxylate

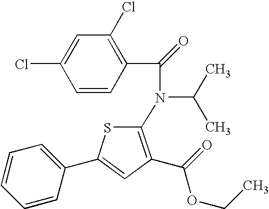

0.72 g (3.46 mmol) of 2,4-dichlorobenzoyl chloride are added under argon to a solution of 0.20 g (0.69 mmol) of ethyl 2-isopropylamino-5-phenylthiophene-3-carboxylate in 50 ml of pyridine. The mixture is heated at 120° C. overnight. It is then concentrated, and the residue is purified by preparative HPLC (RP18 column; mobile phase: acetonitrile-water gradient 95:5→5:95). 0.23 g (70% of theory) of product are obtained.

LC-MS (Method 5): Rₜ=3.19 min

MS (ESIpos): m/z=462 (M+H)⁺.

¹H-NMR (200 MHz, DMSO-d₆): δ=7.67-7.53 (m, 4H), 7.47-7.24 (m, 5H), 4.94 (sept, 1H), 4.34 (q, 2H), 1.36 (d, 3H), 1.34 (t, 3H), 1.02 (d, 3H).

General Procedure [B]: Acylation of 2-Alkylaminothiophenes with Carbonyl Chlorides

Variant 1: Pyridine as Solvent

0.3 mmol (1.0 equivalent) of the 2-alkylaminothiophene and 0.9 mmol (3.0 equivalents) of the acid chloride are introduced into 5 ml of pyridine, and the mixture is stirred at 120° C. for 16 h. After cooling, the mixture is diluted with dichloromethane and extracted three times with 1N hydrochloric acid and once with a saturated sodium chloride solution. The organic phase is dried over sodium sulfate and filtered, and the solvent is removed in vacuo. The product is then purified by preparative HPLC (Method 2) or chromatography on silica gel with cyclohexane/ethyl acetate mixtures.

Variant 2: Use of the Acid Chloride as Solvent

0.5 ml of the acid chloride are mixed with the amine (0.5-1.0 mmol per 1 ml of acid chloride) and, optionally in the presence of acid-labile protective groups such as, for example, tert-butyl esters, 3.0 equivalents of Hünig's base are also added. The mixture is stirred at 80-90° C. overnight. Depending on the conversion, optionally the same amount of acid chloride is again added, and the mixture is again stirred at 80-90° C. overnight. Ethyl acetate is then added to the mixture, the organic phase is extracted twice with a saturated sodium bicarbonate solution, dried over sodium sulfate and filtered, and the solvent is removed in vacuo. Purification takes place in analogy to variant 1.

Variant 3: Acetonitrile as Solvent

0.66 mmol (1.0 equivalent) of the 2-alkylaminothiophene and 1.98 mmol (3.0 equivalents) of the acid chloride are introduced into 10 ml of acetonitrile, and the mixture is stirred at 90° C. for 16 h. Depending on the conversion, optionally the same amount of acid chloride is again added, and the mixture is again stirred at 80-90° C. overnight. After cooling, the mixture is diluted with ethyl acetate and extracted three times with a saturated sodium bicarbonate solution. The organic phase is dried over sodium sulfate and filtered, and the solvent is removed in vacuo. The product is then purified by preparative HPLC or chromatography on silica gel with cyclohexane/ethyl acetate mixtures.

Examples 8A to 15A are prepared in an analogous manner by general procedure [B Variant 1] from the appropriate starting compounds.

| Example | | Prepared | |
|---|---|---|---|
| No. | Structure | from (yield) | Analytical data |
| 8A | 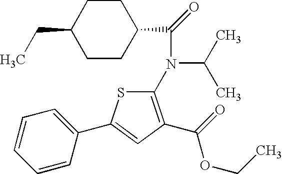 | Example 2A (76% of theory) | LC-MS (Method 4): Rₜ = 3.53 min MS (ESIpos): m/z = 428 (M + H)⁺ |
| 9A | 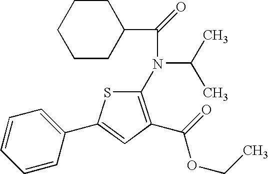 | Example 2A (34% of theory) | LC-MS (Method 3): Rₜ = 3.37 min MS (ESIpos): m/z = 400 (M + H)⁺ |
| 10A | 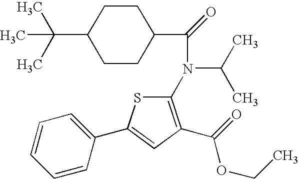 | Example 2A (37% of theory) | LC-MS (Method 3): Rₜ = 3.64 min MS (ESIpos): m/z = 456 (M + H)⁺ |
| 11A | 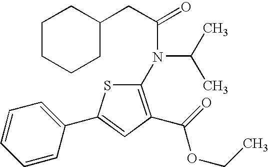 | Example 2A (49% of theory) | LC-MS (Method 3): Rₜ = 3.48 min MS (ESIpos): m/z = 414 (M + H)⁺ |
| 12A | 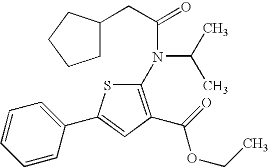 | Example 2A (39% of theory) | LC-MS (Method 3): Rₜ = 3.40 min MS (ESIpos): m/z = 400 (M + H)⁺ |
| 13A | 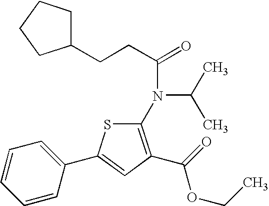 | Example 2A (58% of theory) | LC-MS (Method 4): Rₜ = 3.43 min MS (ESIpos): m/z = 414 (M + H)⁺ |
| 14A | 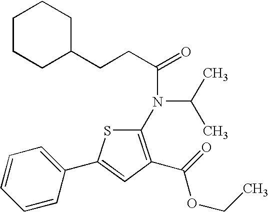 | Example 2A (68% of theory) | LC-MS (Method 5): Rₜ = 3.39 min MS (ESIpos): m/z = 428 (M + H)⁺ |
| 15A | 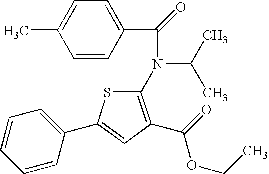 | Example 2A (62% of theory) | LC-MS (Method 5): Rₜ = 3.03 min MS (ESIpos): m/z = 408 (M + H)⁺ |

### Example 16A

### Ethyl 2-[(4-chlorobenzoyl)(cyclohexyl)amino]-5-phenylthiophenecarboxylate

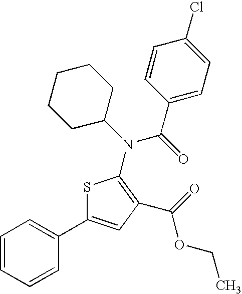

Starting with 100 mg (0.3 mmol) of 2-alkylaminothiophene from Example 4A and 159 mg (0.9 mmol) of 4-chlorobenzoyl chloride, general procedure [B Variant 1], and preparative HPLC (Method 2) result in 80 mg (63% of theory) of product.

HPLC (Method 1): Rₜ=6.17 min

MS (ESI-pos): m/z=468 (M+H)⁺

Examples 17A to 22A are prepared in an analogous manner by general procedure [B Variant 1] from the appropriate starting compounds.

| | | Amine | | |
|---|---|---|---|---|
| | | [amount of amine] | | HPLC |
| Example | | Acid chloride | | Rₜ [min] |
| No. | Structure | [amount of acid chloride] | Yield | (Method) |
| 17A | 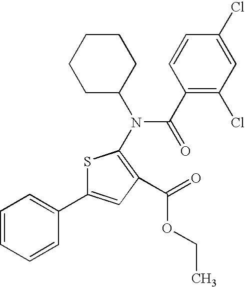 | Example 4A 200 mg (0.61 mmol) 2,4-Dichlorobenzoyl chloride 381 mg(1.8 mmol) | 97 mg (31% of theory) | 6.44 (Method 1) |
| 18A | 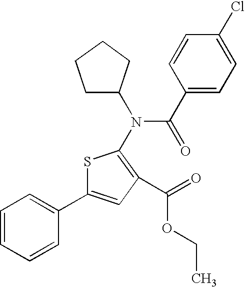 | Example 3A 200 mg (0.63 mmol) 4-Chlorobenzoyl chloride 333 mg (1.9 mmol) | 185 mg (64% of theory) | 5.98 (Method 1) |
| 19A | 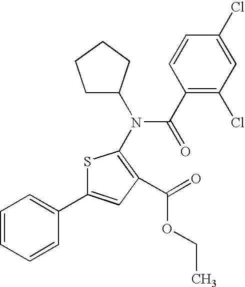 | Example 3A 200 mg (0.63 mmol) 2,4-Dichlorobenzoyl chloride 398 mg(1.9 mmol) | 109 mg (35% of theory) | 6.20 (Method 1) |
| 20A | 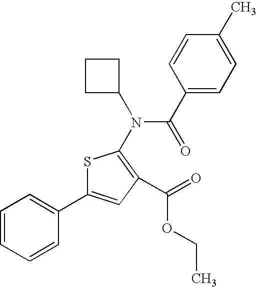 | Example 5A 200 mg (0.66 mmol) 4-Methylbenzoyl chloride 308 mg (1.99 mmol) | 136 mg (49% of theory) | 5.70 (Method 6) |
| 21A | 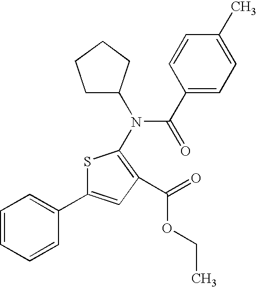 | Example 4A 200 mg (0.63 mmol) 4-Methylbenzoyl chloride 294 mg (1.90 mmol) | 139 mg (51% of theory) | 5.85 (Method 6) |
| 22A | 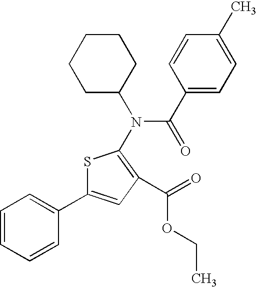 | Example 3A 200 mg (0.61 mmol) 4-Methylbenzoyl chloride 282 mg (1.82 mmol) | 128 mg (47% of theory) | 6.03 (Method 6) |

Example 23A is prepared in an analogous manner by general procedure [A] from the appropriate starting compounds.

| | | Amine | | |
|---|---|---|---|---|
| | | [amount of amine] | | HPLC |
| Example | | Carbonyl compound | | Rₜ [min] |
| No. | Structure | [amount of carbonyl comp.] | Yield | (Method) |
| 23A | 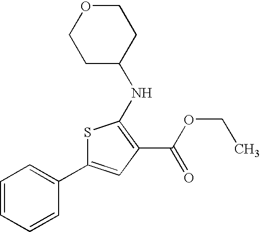 | Example 1A 2.0 g (8.1 mmol) Tetrahydropyran-4-one 3.2 g (32.3 mmol) | 0.69 g (22% of theory) | 5.45 (Method 1) |

Example 24A is prepared in an analogous manner by general procedure [B Variant 1] from the appropriate starting compounds.

| | | Prepared | |
|---|---|---|---|
| Example | | from | |
| No. | Structure | (yield) | Analytical data |
| 24A | 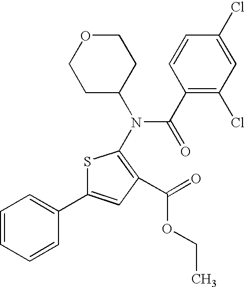 | Example 23A (76% of theory) | LC-MS (Method 6): Rₜ = 5.60 min MS (CIpos): m/z = 521 (M + NH₄)⁺ |

### Example 25A

### Methyl 2-(isopropylamino)thiophene-3-carboxylate

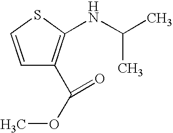

27.5 g (175.0 mmol) of methyl 2-aminothiophene-3-carboxylate are introduced into 533 ml of dichloromethane under argon and, at RT, 50.5 g (700 mmol) of 2-methoxypropene, 42.0 g of glacial acetic acid (700 mmol) and 74.2 g (350 mmol) of sodium triacetoxyborohydride are added. The reaction mixture is stirred at RT for 2 h and then neutralized with an aqueous 2N sodium hydroxide solution. After separation of the phases, the aqueous phase is extracted three times with dichloromethane. The combined organic phases are dried with sodium sulfate, and the solvent is stripped off under reduced pressure with gentle warming on a rotary evaporator. The residue is purified by flash chromatography (mobile phase gradient: 0% ethyl acetate in petroleum ether to 3% ethyl acetate in petroleum ether) to result in 24.9 g (72% of theory) of product.

HPLC (Method 1): Rₜ=4.88 min

MS (DCI(NH₃)): m/z=200 (M+H)⁺.

¹H-NMR (400 MHz, CDCl₃): δ=7.36 (d, 1H), 7.00 (d, 1H), 6.14 (d, 1H), 3.78 (s, 3H), 3.45-3.55 (m, 1H), 1.31 (d, 6H).

### Example 26A

### Methyl 2-{isopropyl [(trans-4-methylcyclohexyl)carbonyl]amino}-thiophene-3-carboxylate

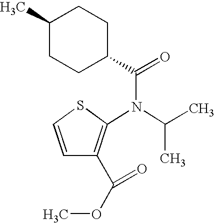

30.0 g (211.0 mmol) of trans-4-methylcyclohexanecarboxylic acid are dissolved in 150.6 g (1.27 mol) of thionyl chloride under argon and boiled under reflux for 1 h. After cooling, the excess thionyl chloride is stripped off in vacuo with gentle heating, and the residue is coevaporated three times with dry toluene. 33.9 g (211.0 mmol) of trans-4-methylcyclohexanecarbonyl chloride and 25.9 g (200.5 mmol) of dry diisopropylethylamine are added to 21.0 g (105.5 mmol) of methyl 2-isopropylaminothiophene-3-carboxylate under argon. The mixture is heated with stirring at 80° C. overnight. The mixture is then diluted with dichloromethane, and a saturated aqueous sodium carbonate solution is cautiously added. The phases are separated, the aqueous phase is extracted three times with dichloromethane, and the combined organic phases are dried over sodium sulfate, filtered and concentrated. Column chromatography on silica gel 60 (mobile phase: ethyl acetate:cyclohexane 1:5) results in 32.5 g (49% of theory) of product.

HPLC (Method 1): Rₜ=5.20 min

MS (ESI+): m/z=324 (M+H)⁺.

¹H-NMR (400 MHz, CDCl₃): δ=7.44 (d, 1H), 7.19 (d, 1H), 4.94 (m, 1H), 3.79 (s, 3H), 1.93-2.09 (m, 1H), 1.56-1.78 (m, 5H), 1.19-1.43 (m, 2H), 1.16 (d, 3H), 0.88 (d, 3H), 0.77 (d, 3H), 0.54-0.74 (m, 2H).

### Example 27A

### Methyl 5-bromo-2-{isopropyl [(trans-4-methylcyclohexyl)carbonyl]-amino l thiophene-3-carboxylate

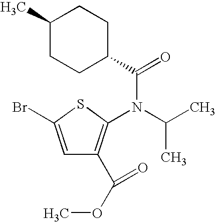

15.4 g (47.1 mmol) of methyl 2-{isopropyl[(trans-4-methylcyclohexyl)carbonyl]amino}thiophene-3-carboxylate are introduced into 308 ml of a 1:1 chloroform:tetrachloromethane mixture, and 21.2 g (119.0 mmol) of N-bromosuccinimide are added. The mixture is stirred under reflux for 2.5 h. After cooling, the solvent is removed in vacuo with gentle heating. The residue is purified by flash chromatography on silica gel 60 (solvent: ethyl acetate:cyclohexane 1:5) to result in 13.8 g (72% of theory) of product.

HPLC (Method 6): Rₜ=5.88 min

MS (ESI+): m/z=402 (M+H)⁺.

¹H-NMR (400 MHz, CDCl₃): δ=7.42 (s, 1H), 4.91 (m, 1H), 3.78 (s, 3H), 2.02-2.16 (m, 1H), 1.55-1.74 (m, 5H), 1.22-1.49 (m, 2H), 1.14 (d, 3H), 0.90 (d, 3H), 0.79 (d, 3H), 0.61-0.78 (m, 2H).

### Example 28A

### Methyl 5-(4-fluorophenyl)-2-{isopropyl[(trans-4-methylcyclohexyl)carbonyl]amino}thiophene-3-carboxylate

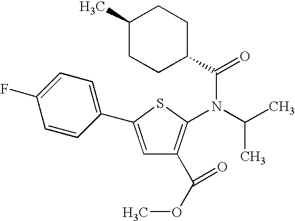

200.0 mg (0.5 mmol) of methyl 5-bromo-2-{isopropyl[(trans-4-methylcyclohexyl)carbonyl]amino}thiophene-3-carboxylate from Example 27A are dissolved in 4 ml of dry N,N′-dimethylformamide under argon, and 208.7 mg (1.49 mmol) of 4-fluorophenylboronic acid and 546.8 μl of an aqueous 2N sodium carbonate solution are added. Argon is passed through the reaction solution at 80° C. for 1 h. Then 40.59 mg (0.05 mmol) of bis[(diphenylphosphino)ferrocene]palladium(II) chloride are added as catalyst, and the mixture is stirred at 80° C. for 18 h. After cooling, the reaction solution is filtered through Celite® and washed with ethyl acetate. The combined organic phases are stripped off in vacuo with gentle warming. Preparative HPLC (RP-18 column, mobile phase: acetonitrile-water gradient) results in 181 mg (87% of theory) of product.

HPLC (Method 6): Rₜ=5.89 min

MS (DCI(NH₃)): m/z=418 (M+NH₄)⁺.

¹H-NMR (400 MHz, DMSO-d₆): δ=7.75-7.86 (m, 3H), 7.25-7.33 (m, 2H), 4.79 (m, 1H), 3.77 (s, 3H), 2.06-2.17 (m, 1H), 1.44-1.72 (m, 5H), 1.20-1.35 (m, 2H), 1.14 (d, 3H), 0.87 (d, 3H), 0.75 (d, 3H), 0.53-0.72 (m, 2H).

### Example 29A

### Methyl 5-(2,4-difluorophenyl)-2-{isopropyl[(trans-4-methylcyclohexyl)carbonyl]amino}thiophene-3-carboxylate

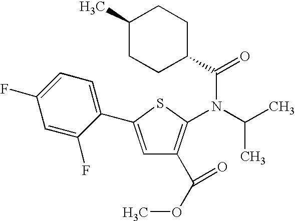

200.0 mg (0.5 mmol) of methyl 5-bromo-2-{isopropyl[(trans-4-methylcyclohexyl)carbonyl]amino}thiophene-3-carboxylate from Example 27A are dissolved in 2 ml of N,N′-dimethylformamide under argon, and 176.6 mg (1.12 mmol) of 2,4-difluorophenylboronic acid and 410 μl of an aqueous 2N sodium carbonate solution are added. Argon is passed through the reaction solution at 80° C. for 1 h. Then bis[(diphenylphosphino)ferrocene]palladium(II) chloride is added as catalyst, and the mixture is stirred at 80° C. for 20 h. After cooling, the reaction solution is filtered and, without further purification, purified by preparative HPLC (RP-18 column, mobile phase: acetonitrile-water gradient). 123 mg (76% of theory) of product are obtained.

HPLC (Method 1): Rₜ=6.09 min

MS (ES+): m/z=436 (M+H)⁺.

¹H-NMR (400 MHz, CDCl₃): δ=7.73 (s, 1H), 7.56-7.65 (m, 1H), 6.90-7.01 (m, 2H), 4.92-5.04 (m, 1H), 3.83 (s, 3H), 2.10-2.21 (m, 1H), 1.58-1.79 (m, 5H), 1.27-1.51 (m, 2H), 1.22 (d, 3H), 0.95 (d, 3H), 0.79 (d, 3H), 0.61-0.77 (m, 2H).

General Procedure [D]: Suzuki Coupling of 5-bromothiophenes with Boronic Acids and Boronic Ester

2.97 mmol (1.0 equivalent) of the 5-bromothiophene are introduced into 19.0 ml of dry dimethylformamide under argon, and 8.92 mmol (3 equivalents) of the boronic acid or of the boronic ester and 3.27 ml (2.2 equivalents) of an aqueous 2N sodium carbonate solution are added. Argon is passed through the reaction solution at 80° C. for 1 h. Then 0.30 mmol (0.1 equivalents) of bis[(diphenylphosphino)ferrocene]palladium(II) chloride are added as catalyst, and the mixture is stirred at 80° C. for 18 h. After cooling, the reaction solution is filtered through Celite® and washed with ethyl acetate. The combined organic phases are stripped off in vacuo with gentle heating. The residue is purified by column chromatography on silica gel 60 (mobile phase: gradient of ethyl acetate in cyclohexane). Alternatively, purification is possible by preparative HPLC (RP-18 column, mobile phase: acetonitrile-water gradient).

Examples 30A to 37A are prepared in an analogous manner by general procedure [D] from the appropriate starting compounds.

| | | [Amount of bromothiophene] | |
|---|---|---|---|
| | | Boronic acid | Analytical data |
| Example | | [amount of boronic acid] | HPLC (Method) |
| No. | Structure | (yield) | MS (Method) |
| 30A | 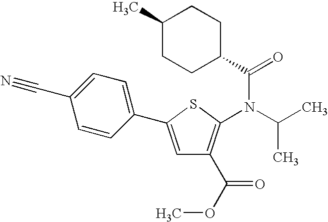 | 150 mg (0.373 mmol) 4-Cyanoboronic acid 164.3 mg (1.12 mmol) (54% of theory) | HPLC (Method 6): Rₜ = 5.57 min MS (ES+): m/z = 425 (M + H)⁺ |
| 31A | 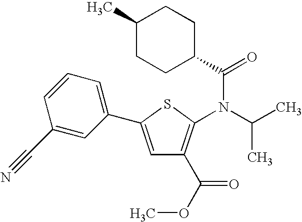 | 150 mg (0.373 mmol) 3-Cyanophenylboronic acid 164.3 mg (1.12 mmol) (63% of theory) | HPLC (Method 6): Rₜ = 5.58 min MS (ES+): m/z = 425 (M + H)⁺ |
| 32A | 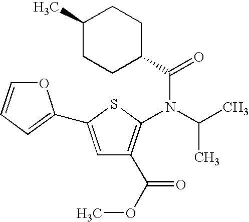 | 120 mg (0.298 mmol) 2-Furanboronic acid 100.1 mg (0.89 mmol) (64% of theory) | HPLC (Method 6): Rₜ = 5.48 min MS (DCI(NH₃)): m/z = 390 (M + H)⁺ |
| 33A | 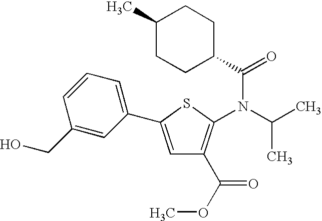 | 150 mg (0.373 mmol) 3-Hydroxymethylphenylboronic acid 170.0 mg (1.12 mmol) (59% of theory) | HPLC (Method 6): Rₜ = 5.11 min MS (ES+): m/z = 430 (M + H)⁺ |
| 34A | 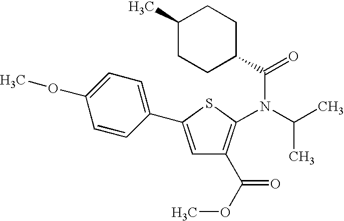 | 150 mg (0.373 mmol) 4-Methoxyphenylboronic acid 169.95 mg (1.12 mmol) (61% of theory) | HPLC (Method 6): Rₜ = 5.92 min MS (DCI(NH₃)): m/z = 430 (M + H)⁺ |
| 35A | 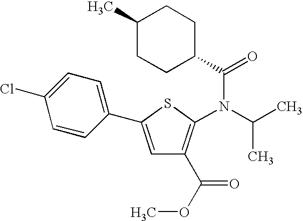 | 150 mg (0.373 mmol) 4-Chlorophenylboronic acid 174.9 mg (1.12 mmol) (59% of theory) | HPLC (Method 6): Rₜ = 6.54 min MS (ES+): m/z = 434 (M + H)⁺ |
| 36A | 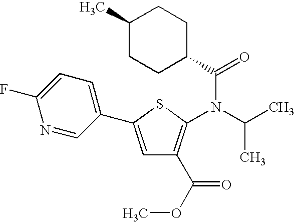 | 150 mg (0.373 mmol) 2-Fluoropyridine-5-boronic acid 157.6 mg (1.12 mmol) (63% of theory) | HPLC (Method 1): Rₜ = 5.44 min MS (ES+): m/z = 419 (M + H)⁺ |
| 37A | 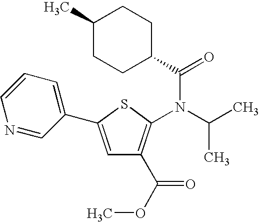 | 40 mg (0.099 mmol) 3-Pyridylboronic acid 36.7 mg (0.30 mmol) (56% of theory) | HPLC (Method 6): Rₜ = 4.32 min MS (ES+): m/z = 401 (M + H)⁺ |

### Example 38A

### Methyl 2-{isopropyl[(trans-4-methylcyclohexyl)carbonyl]amino}-5-(1,3-thiazol-2-yl)thiophene-3-carboxylate

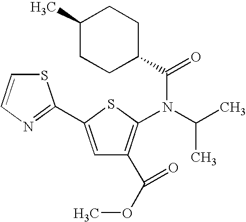

588.5 mg (9.0 mmol) of zinc dust are suspended in 1.5 ml of dry THF under argon, and 152.2 mg (0.81 mmol) of 1,2-dibromoethane are added. The mixture is heated under reflux for 2 h. After cooling, 39.1 mg (0.36 mmol) of chlorotrimethylsilane and a solution of 492.1 mg (3.0 mmol) of 2-bromothiazol in 1.2 ml of dry THF are added, and the mixture is heated under reflux for 1 h. 1.81 g (4.5 mmol) of methyl 5-bromo-2-{isopropyl[(trans-4-methylcyclohexyl)carbonyl]amino}thiophene-3-carboxylate and 69.3 mg (0.06 mmol) of tetrakis(triphenylphosphine)palladium(0) are dissolved in 6 ml of THF and added to the reaction mixture. After heating under reflux for 18 hours, the mixture is allowed to cool, 3 ml of water are added, and the mixture is concentrated. The residue is filtered and purified by preparative HPLC (RP-18 column, mobile phase, acetonitrile-water gradient). For the final purification, the compound obtained in this way is again purified by preparative HPLC (Reprosil ODS-A. 5 μm 250×20 mm, 25 ml/min, acetonitrile-water 70-30). 85 mg (7% of theory) of product are obtained.

HPLC (Method 6): Rₜ=5.33 min

MS (DCI(NH₃)): m/z=407 (M+H)⁺.

¹H-NMR (400 MHz, DMSO-d₆): δ=7.84-7.92, 4.80 (m, 1H), 3.77 (s, 3H), 2.02-2.18 (m, 1H), 1.40-1.73 (m, 5H), 1.18-1.38 (m, 2H), 1.14 (d, 3H), 0.87 (d, 3H), 0.75 (d, 3H), 0.51-0.72 (m, 2H).

The following compound was synthesized in analogy to the synthesis of Example 97A:

| Example | | Starting compounds | |
|---|---|---|---|
| No. | Structure | (yield) | Analytical data |
| 39A | 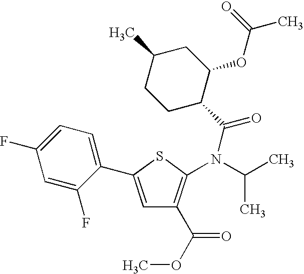 | 221 mg (0.49 mmol) Example 96A, (47 mg (19% of theory)) | HPLC (Method 1): Rₜ = 5.69 min MS (DCI): m/z = 494 (M + H)⁺ |

### Example 40A

### Methyl 2-[(tert-butoxycarbonyl)amino]thiophene-3-carboxylate

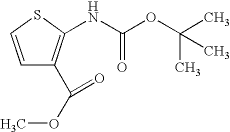

10.0 g (63.6 mmol) of methyl 2-aminothiophene-3-carboxylate and a catalytic amount of 4-dimethylaminopyridine are dissolved in 200 ml of dichloromethane. 20.8 g (95.4 mmol) of di-tert-butyl pyrocarbonate are cautiously added, and the mixture is stirred at RT for 18 h. The solvent is stripped off in vacuo with gentle heating. Column chromatography on silica gel 60 (mobile phase: gradient from 0% ethyl acetate in cyclohexane to 5% ethyl acetate in cyclohexane) results in 8.16 g (50% of theory) of product.

HPLC (Method 1): R=5.09 min

MS (DCI(NH₃)): m/z=258 (M+H)⁺.

¹H-NMR (400 MHz, CDCl₃): δ=10.0 (br. s, 1H), 7.15 (d, 1H), 6.64 (d, 1H), 3.86 (s, 3H), 1.53 (s, 9H).

### Example 41A

### Methyl 2-[(tert-butoxycarbonyl)amino]-5-bromothiophene-3-carboxylate

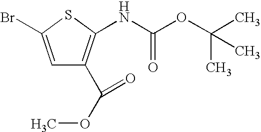

4.6 g (17.7 mmol) of methyl 2-[(tert-butoxycarbonyl)amino]thiophene-3-carboxylate and 3.5 g (19.5 mmol) of N-bromosuccinimide are dissolved in 228 ml of a 1:1 tetrachloromethane:chloroform mixture and heated under reflux for 2 h. After cooling, the solvent is stripped off in vacuo with gentle heating, and the residue is purified by flash chromatography (solvent: dichloromethane). 4.8 g (79% of theory) of product are obtained.

HPLC (Method 1): Rₜ=5.62 min

MS (DCI(NH₃)): m/z=353 (M+NH₄)⁺.

¹H-NMR (400 MHz, CDCl₃): δ=10.0 (br. s, 1H), 7.12 (s, 1H), 3.85 (s, 3H), 1.53 (s, 9H).

### Example 42A

### Methyl 2-[(tert-butoxycarbonyl)amino]-5-(4-fluorophenyl)thiophene-3-carboxylate

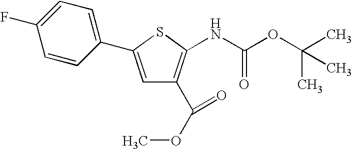

1.00 g (2.97 mmol) of methyl 2-[(tert-butoxycarbonyl)amino]-5-bromothiophene-3-carboxylate from Example 41A are dissolved in 19 ml of dry N,N-dimethylformamide under argon, and 1.25 g (8.92 mmol) of 4-fluorophenylboronic acid and 3.27 ml of an aqueous 2N sodium carbonate solution are added. Argon is passed through the reaction solution at 80° C. for 1 h. Then 217.64 mg (0.297 mmol) of bis[(diphenylphosphino)ferrocene]palladium(II) chloride are added as catalyst, and the mixture is stirred at 80° C. for 18 h. After cooling, the reaction solution is filtered through Celite® and washed with ethyl acetate. The combined organic phases are stripped off in vacuo with gentle heating. The residue is purified by column chromatography on silica gel 60 (mobile phase: gradient of ethyl acetate in cyclohexane) to result in 735 mg (68% of theory) of product.

HPLC (Method 1): Rₜ=5.94 min

MS (DCI(NH₃)): m/z=369 (M+NH₄)⁺.

¹H-NMR (400 MHz, CDCl₃): δ=10.0 (br. s, 1H), 7.46-7.55 (m, 2H), 7.29 (s, 1H), 6.99-7.10 (m, 2H), 3.89 (s, 3H), 1.55 (s, 9H).

### Example 43A

### Methyl 2-[(tert-butoxycarbonyl)amino]-5-(3,4-difluorophenyl)thiophene-3-carboxylate

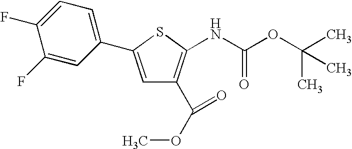

1.00 g (2.97 mmol) of methyl 2-[(tert-butoxycarbonyl)amino]-5-bromothiophene-3-carboxylate from Example 41A and 1.41 g (8.92 mmol) of 3,4-difluorophenylboronic acid are reacted according to general procedure [D] to result, after flash chromatography on silica gel 60 (mobile phase: gradient 0% ethyl acetate in cyclohexane to 2% ethyl acetate in cyclohexane), in 708 mg (65% of theory) of product.

HPLC (Method 1): Rₜ=6.02 min

MS (DCI(NH₃)): m/z=387 (M+NH₄)⁺.

¹H-NMR (400 MHz, CDCl₃): δ=10.0 (br. s, 1H), 7.06-7.40 (m, 4H), 3.89 (s, 3H), 1.55 (s, 9H).

### Example 44A

### Methyl 2-[(tert-butoxycarbonyl)amino]-5-(2,4-difluorophenyl)-thiophene-3-carboxylate

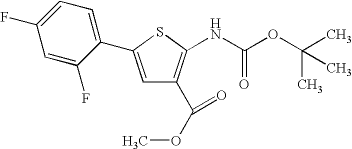

1.00 g (2.97 mmol) of methyl 2-[(tert-butoxycarbonyl)amino]-5-bromothiophene-3-carboxylate from Example 41A and 1.41 g (8.92 mmol) of 2,4-difluorophenylboronic acid are reacted according to general procedure [D] to result, after flash chromatography on silica gel 60 (mobile phase: gradient 0% ethyl acetate in cyclohexane to 2% ethyl acetate in cyclohexane), in 597 mg (52% of theory) of product.

HPLC (Method 6): Rₜ=6.02 min

MS (DCI(NH₃)): m/z=387 (M+NH₄)⁺.

¹H-NMR (400 MHz, CDCl₃): δ=10.07 (br. s, 1H), 7.39-7.58 (m, 2H), 6.80-6.96 (m, 2H), 3.89 (s, 3H), 1.55 (s, 9H).

### Example 45A

### Methyl 2-amino-5-(4-fluorophenyl)thiophene-3-carboxylate

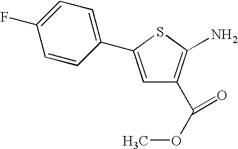

29.4 ml of a solution of 4N hydrochloric acid in dioxane are added to 687.5 mg (1.96 mmol) of methyl 2-[(tert-butoxycarbonyl)amino]-5-(4-fluorophenyl)thiophene-3-carboxylate from Example 42A and stirred at room temperature overnight. Then a saturated aqueous sodium carbonate solution is cautiously added, the phases are separated, the aqueous phase is extracted three times with ethyl acetate, and the combined organic phases are dried over sodium sulfate, filtered and concentrated. Column chromatography on silica gel 60 (solvent: gradient 0% ethyl acetate in cyclohexane to 20% ethyl acetate in cyclohexane) results in 312 mg (63% of theory) of product.

HPLC (Method 6): Rₜ=4.70 min

MS (ES+): m/z=252 (M+H)⁺.

¹H-NMR (400 MHz, CDCl₃): δ=7.34-7.43 (m, 2H), 7.14 (s, 1H), 6.96-7.07 (m, 2H), 5.97 (br. s, 2H), 3.83 (s, 3H).

General Procedure [E]: Removal of the tert-butoxycarbonyl Protecting Group from Methyl 2-[(tert-butoxycarbonyl)(isopropyl)amino]-5-arylthiophene-3-carboxylates with Hydrochloric Acid in Dioxane

110.7 mmol (60 equivalents) of a solution of 4N hydrochloric acid in dioxane are added to 1.85 mmol (1.0 equivalent) of the methyl 2-[(tert-butoxycarbonyl)amino]-5-arylthiophene-3-carboxylate at room temperature. The mixture is stirred at RT overnight and then quenched with a saturated aqueous sodium carbonate solution. After phase separation, the aqueous phase is extracted three times with ethyl acetate. The combined organic phases are dried over sodium sulfate and filtered, and the solvent is removed in vacuo. The product is then purified by flash chromatography on silica gel 60 (solvent: gradient 0% ethyl acetate in cyclohexane to 20% ethyl acetate in cyclohexane).

### Example 46A

### Methyl 2-amino-5-(3,4-difluorophenyl)thiophene-3-carboxylate

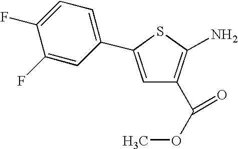

681.6 mg (1.85 mmol) of methyl 2-[(tert-butoxycarbonyl)amino]-5-(3,4-difluorophenyl)thiophene-3-carboxylate from Example 43A and 27.7 ml of a solution of 4N hydrochloric acid in dioxane are reacted according to general procedure [E] to result, after flash chromatography on silica gel 60 (solvent: gradient 0% ethyl acetate in cyclohexane to 20% ethyl acetate in cyclohexane), in 391 mg (79% of theory) of product.

HPLC (Method 6): Rₜ=4.80 min

MS (ES+): m/z=270 (M+H)⁺.

¹H-NMR (400 MHz, CDCl₃): δ=7.07-7.27 (m, 4H), 6.00 (br. s, 2H), 3.84 (s, 3H).

### Example 47A

### Methyl 2-amino-5-(2,4-difluorophenyl)thiophene-3-carboxylate

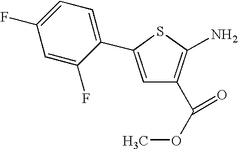

590 mg (1.60 mmol) of methyl 2-[(tert-butoxycarbonyl)amino]-5-(2,4-difluorophenyl)thiophene-3-carboxylate from Example 44A and 24.0 ml of a solution of 4N hydrochloric acid in dioxane are reacted according to general procedure [E] to result, after flash chromatography on silica gel 60 (solvent: gradient 0% ethyl acetate in cyclohexane to 20% ethyl acetate in cyclohexane), in 324.7 mg (76% of theory) of product.

HPLC (Method 1): Rₜ=4.74 min

MS (ES+): m/z=270 (M+H)⁺.

¹H-NMR (400 MHz, DMSO-d₆): δ=7.44-7.66 (m, 3H), 7.21-7.36 (m, 2H), 7.02-7.13 (m, 1H), 3.73 (s, 3H).

### Example 48A

### Methyl 5-(4-fluorophenyl)-2-(tetrahydro-2H-pyran-4-ylamino)-thiophene-3-carboxylate

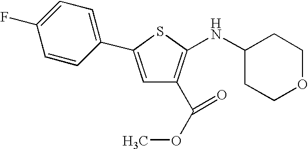

0.66 ml of a solution of 1 M titanium (IV) tetrachloride in dichloromethane are added to a solution of 150 mg (0.60 mmol) of methyl 2-amino-5-(4-fluorophenyl)thiophene-3-carboxylate and 83.7 mg (0.84 mmol) of tetrahydro-4H-pyran-4-one in 2.0 ml of dry dichloromethane at −78° C. under argon. The mixture is stirred at 0° C. for 3 h and, after the addition of 379.6 mg (1.79 mmol) of sodium triacetoxyborohydride, stirred at RT for 18 h. A few drops of methanol are added, and the mixture is diluted with dichloromethane and a saturated aqueous sodium carbonate solution is added. After phase separation, the aqueous phase is extracted three times with dichloromethane. The combined organic phases are dried over sodium sulfate, filtered and concentrated. Preparative HPLC (RP-18 column, mobile phase: acetonitrile-water gradient) results in 99.1 mg (50% of theory) of product.

HPLC (Method 6): Rₜ=5.19 min

MS (DCI(NH₃)): m/z=336 (M+H)⁺.

¹H-NMR (400 MHz, CDCl₃): δ=7.58 (d, 1H), 7.36-7.43 (m, 2H), 7.19 (s, 1H), 6.98-7.06 (m, 2H), 3.98-4.06 (m, 2H), 3.82 (s, 3H), 3.49-3.58 (m, 2H), 3.38-3.49 (m, 1H), 2.08-2.16 (m, 2H), 1.61-1.73 (m, 2H).

General Procedure [F]: Reductive Amination of 2-Aminothiophenes with Aldehydes and Ketones

Reaction with Cyclic Ketones without Dehydrating Reagents

0.56 mmol (1.0 equivalent) of the 2-aminothiophene are introduced into 2.0 ml of dry dichloromethane under argon and, at room temperature, 0.78 mmol (1.4 equivalents) of the carbonyl compound are added. The mixture is cooled to −78° C., and 0.61 mmol (1.1 equivalents) of a 1 M titanium (IV) tetrachloride solution in dichloromethane are slowly added dropwise. The mixture is stirred at 0° C. for 3 h. Then 1.67 mmol (3 equivalents) of sodium triacetoxyborohydride are added, and the solution is stirred at RT for 18 h. The reaction is quenched with a few drops of methanol, diluted with dichloromethane, and a saturated aqueous sodium carbonate solution is added. After phase separation, the aqueous phase is extracted three times with dichloromethane. The combined organic phases are washed once with a saturated sodium chloride solution, dried over sodium sulfate and filtered, and the solvent is removed in vacuo. The product is then purified by flash chromatography on silica gel or by preparative HPLC.

### Example 49A

### Methyl 5-(3,4-difluorophenyl)-2-(tetrahydro-2H-pyran-4-ylamino)-thiophene-3-carboxylate

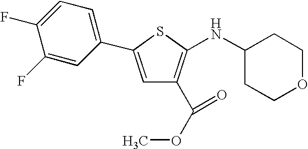

150 mg (0.56 mmol) of methyl 2-amino-5-(3,4-difluorophenyl)thiophene-3-carboxylate are introduced into 2 ml of dry dichloromethane under argon and reacted with 78.1 mg (0.78 mmol) of tetrahydro-4H-pyran-4-one according to general procedure [F]. Preparative HPLC (RP-18 column, mobile phase: acetonitrile-water gradient) results in 164 mg (75% of theory) of product.

HPLC (Method 6): Rₜ=5.27 min

MS (ESI+): m/z=354 (M+H)⁺.

¹H-NMR (400 MHz, CDCl₃): δ=7.62 (d, 1H), 7.19-7.24 (m, 2H), 7.06-7.15 (m, 2H), 3.99-4.07 (m, 2H), 3.82 (s, 3H), 3.49-3.59 (m, 2H), 3.27-3.49 (m, 1H), 2.07-2.16 (m, 2H), 1.61-1.73 (m, 2H).

### Example 50A

### Methyl 5-(2,4-difluorophenyl)-2-(tetrahydro-2H-pyran-4-ylamino)-thiophene-3-carboxylate

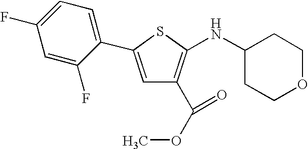

150 mg (0.56 mmol) of methyl 2-amino-5-(2,4-difluorophenyl)thiophene-3-carboxylate are introduced into 2 ml of dry dichloromethane under argon and reacted with 78.1 mg (0.78 mmol) of tetrahydro-4H-pyran-4-one according to general procedure [F]. Preparative HPLC (RP-18 column, mobile phase: acetonitrile-water gradient) results in 65.4 mg (33% of theory) of product.

HPLC (Method 6): Rₜ=5.26 min

MS (DCI(NH₃)): m/z=354 (M+H)⁺.

¹H-NMR (400 MHz, CDCl₃): δ=7.58-7.65 (d, 1H), 7.37-7.46 (m, 1H), 7.36 (s, 1H), 6.82-6.91 (m, 2H), 3.98-4.06 (m, 2H), 3.82 (s, 3H), 3.49-3.59 (m, 2H), 3.40-3.49 (m, 1H), 2.07-2.17 (m, 2H), 1.61-1.73 (m, 2H).

### Example 51A

### Methyl 5-(4-fluorophenyl)-2-[[(trans-4-methylcyclohexyl)-carbonyl](tetrahydro-2H-pyran-4-yl)amino]thiophene-3-carboxylate

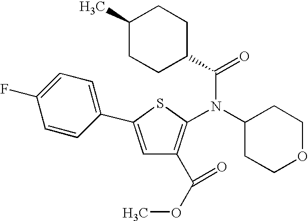

500 μl of trans-4-methylcyclohexanecarbonyl chloride are added to 130.80 mg (0.39 mmol) of methyl 5-(4-fluorophenyl)-2-(tetrahydro-2H-pyran-4-ylamino)thiophene-3-carboxylate under argon, and the mixture is stirred at 80° C. for 20 min. After the addition of 151.2 mg (1.17 mmol) of diisopropylethylamine the mixture is stirred at 80° C. for a further 4 h. Then a further 500 μl of trans-4-methylcyclohexanecarbonyl chloride are added, and the mixture is stirred at 80° C. overnight. After cooling, the mixture is diluted with dichloromethane, and a saturated aqueous sodium carbonate solution is cautiously added. The phases are separated, the aqueous phase is extracted three times with dichloromethane, and the combined organic phases are dried over sodium sulfate, filtered and concentrated. Preparative HPLC (RP-18 column, mobile phase: acetonitrile-water gradient) results in 104.1 mg (58% of theory) of product as a cis/trans mixture.

HPLC (Method 6): Rₜ=5.65 min

MS (DCI(NH₃)): m/z=460 (M+H)⁺.

¹H-NMR (400 MHz, CDCl₃): δ=7.51-7.62 (m, 3H), 7.06-7.18 (m, 2H), 4.75-4.90 (m, 1H), 3.86-4.04 (m, 2H), 3.82 (s, 3H), 3.40-3.58 (m, 2H), 2.11-2.24 (m, 1H), 1.21-1.91 (m, 11H), 0.60-0.83 (m, 5H).

### Example 52A

### Methyl 5-(3,4-difluorophenyl)-2-[[(trans-4-methylcyclohexyl)carbonyl](tetrahydro-2H-pyran-4-yl)amino]thiophene-3-carboxylate

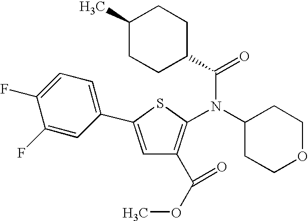

500 μl of trans-4-methylcyclohexanecarbonyl chloride are added to 215.6 mg (0.61 mmol) of methyl 5-(3,4-difluorophenyl)-2-(tetrahydro-2H-pyran-4-ylamino)thiophene-3-carboxylate under argon and the mixture is stirred at 80° C. for 20 min. After the addition of 236.5 mg (1.83 mmol) of diisopropylethylamine the mixture is stirred at 80° C. for a further 4 h. Then a further 500 μl of trans-4-methylcyclohexanecarbonyl chloride are added, and the mixture is stirred at 80° C. overnight. After cooling, the mixture is diluted with dichloromethane, and a saturated aqueous sodium carbonate solution is cautiously added. The phases are separated, the aqueous phase is extracted three times with dichloromethane, and the combined organic phases are dried over sodium sulfate, filtered and concentrated. Preparative HPLC (RP-18 column, mobile phase: acetonitrile-water gradient) results in 164.2 mg (56% of theory) of product as a cis/trans mixture.

HPLC (Method 6): Rₜ=5.69 min

MS (DCI(NH₃)): m/z=478 (M+H)⁺.

### Example 53A

### Methyl 5-(2,4-difluorophenyl)-2-[[(trans-4-methylcyclohexyl)-carbonyl](tetrahydro-2H-pyran-4-yl)amino]thiophene-3-carboxylate

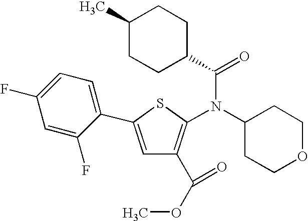

500 μl of trans-4-methylcyclohexanecarbonyl chloride are added to 84.8 mg (0.24 mmol) of methyl 5-(2,4-difluorophenyl)-2-(tetrahydro-2H-pyran-4-ylamino)thiophene-3-carboxylate under argon, and the mixture is stirred at 80° C. for 20 min. After the addition of 93.1 mg (0.72 mmol) of diisopropylethylamine the mixture is stirred at 80° C. for a further 4 h. Then a further 500 μl of trans-4-methylcyclohexanecarbonyl chloride are added, and the mixture is stirred at 80° C. overnight. After cooling, the mixture is diluted with dichloromethane, and a saturated aqueous sodium carbonate solution is cautiously added. The phases are separated, the aqueous phase is extracted three times with dichloromethane, and the combined organic phases are dried over sodium sulfate, filtered and concentrated. Preparative HPLC (RP-18 column, mobile phase: acetonitrile-water gradient) results in 72.8 mg (64% of theory) of product as a cis/trans mixture.

HPLC (Method 6): Rₜ=5.71 min

MS (DCI(NH₃)): m/z=478 (M+H)⁺.

¹H-NMR (400 MHz, CDCl₃): δ=7.72 (s, 1H), 7.55-7.66 (m, 1H), 6.90-7.08 (m, 2H), 4.76-4.90 (m, 1H), 3.87-4.04 (m, 2H), 3.83 (s, 3H), 3.40-3.57 (m, 2H), 2.06-2.23 (m, 1H), 1.18-1.95 (m, 1H), 0.58-0.83 (m, 5H).

### Example 54A

### Methyl 2-aminothiophene-3-carboxylate

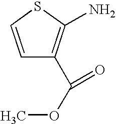

In analogy to J. Med. Chem. 1999, 42, 5437-5447, 65.1 g (656.9 mmol) of methyl cyanoacetate and 50.0 g (328.4 mmol) of 2,5-dihydroxy-1,4-dithiane are introduced into 400 ml of DMF and, while cooling in ice, 45.8 ml (328.4 mmol) of triethylamine are slowly added dropwise. The reaction mixture is stirred at RT for 2 h and then diluted with 1.21 of 0.4M acetic acid. The aqueous phase is extracted four times with 150 ml of tert-butyl methyl ether each time. The combined organic phases are washed twice with 150 ml of water each time and dried with sodium sulfate. The solvent is removed under reduced pressure with gentle heating on a rotary evaporator. The residue is purified by washing twice with cold cyclohexane to result in 35.3 g (68% of theory, 48% pure) product.

HPLC (Method 1): Rₜ=3.63 min

MS (DCI(NH₃)): m/z=158 (M+H)⁺.

¹H-NMR (400 MHz, CDCl₃): δ=6.96 (d, 1H), 6.18 (d, 1H), 5.94 (br. s, 2H), 3.81 (s, 3H).

### Example 55A

### tert-Butyl 4-[5-amino-4-(methoxycarbonyl)-2-thienyl]piperidine-1-carboxylate

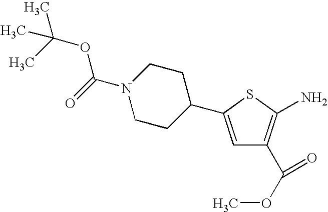

1.0 g (4.40 mmol) of N-Boc-piperidinyl-4-acetaldehyde, 435.9 mg (4.40 mmol) of methyl cyanoacetate and 141.1 mg (4.40 mmol) of sulfur are suspended in methanol and heated to 60° C. 3.0 g (34.4 mmol) of morpholine are slowly added dropwise, and the mixture is stirred at 60° C. for a further 4 h. After cooling, the mixture is concentrated in vacuo with gentle heating and then purified by flash chromatography on silica gel 60 (mobile phase: 20% ethyl acetate in cyclohexane). 1.2 g (82% of theory) of product are obtained.

HPLC (Method 6): Rₜ=4.79 min

MS (ES+): m/z=341 (M+H)⁺.

¹H-NMR (400 MHz, CDCl₃): δ=6.64 (s, 1H), 5.84 (br. S, 2H), 4.15 (d, 2H), 3.78 (s, 3H), 2.78 (t, 2H), 2.62-2.73 (m, 1H), 1.82-1.95 (m, 2H), 1.46-1.59 (m, 2H), 1.46 (s, 9H).

### Example 56A

### tert-Butyl 4-[5-(isopropylamino)-4-(methoxycarbonyl)-2-thienyl]-piperidine-1-carboxylate

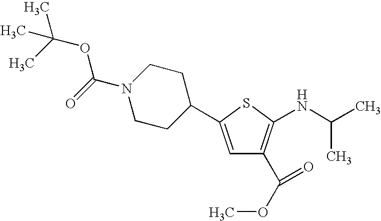

1.2 g (3.53 mmol) tert-butyl 4-[5-amino-4-(methoxycarbonyl)-2-thienyl]piperidine-1-carboxylate are introduced into 10 ml of dichloromethane under argon and, at RT, 1.02 g (14.1 mmol) of 2-methoxypropene, 0.85 g of glacial acetic acid (14.1 mmol) and 1.5 g (7.1 mmol) of sodium triacetoxyborohydride are added. The reaction mixture is stirred at RT for 1 h and then neutralized with a saturated aqueous sodium carbonate solution. After separating the phases, the aqueous phase is extracted three times with dichloromethane. The combined organic phases are dried over sodium sulfate and the solvent is stripped off under reduced pressure with gentle heating. The residue is purified by flash chromatography (mobile phase: gradient 0% ethyl acetate in cyclohexane to 20% ethyl acetate in cyclohexane), resulting in 1.25 g (93% of theory) of product.

HPLC (Method 1): Rₜ=5.59 min

MS (DCI(NH₃)): m/z=383 (M+H)⁺.

¹H-NMR (400 MHz, CDCl₃): δ=7.27 (d, 1H), 6.67 (s, 1H), 4.05-4.24 (m, 2H), 3.76 (s, 3H), 3.40-3.53 (m, 1H), 2.64-2.87 (m, 3H), 1.85-1.95 (m, 2H), 1.48-1.66 (m, 2H), 1.46 (s, 9H), 1.29 (d, 6H).

### Example 57A

### tert-Butyl 4-[5-{isopropyl[(trans-4-methylcyclohexyl)carbonyl]-amino}-4-(methoxycarbonyl)-2-thienyl]pi peridine-1-carboxylate

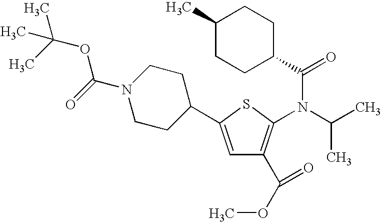

8.0 g (56.3 mmol) of trans-4-methylcyclohexanecarboxylic acid are dissolved in 40.2 g (337.6 mmol) of thionyl chloride under argon and heated under reflux for 2 h. After cooling, the excess thionyl chloride is stripped off in vacuo with gentle heating, and the residue is coevaporated with dry toluene three times. 1.25 g (3.27 mmol) of tert-butyl 4-[5-(isopropylamino)-4-(methoxycarbonyl)-2-thienyl]piperidine-1-carboxylate are dissolved in 6 ml of dry pyridine under argon, and a catalytic amount of 4-dimethylaminopyridine and 1.57 g (9.80 mmol) of trans-4-methylcyclohexanecarbonyl chloride are added. The mixture is heated at 70° C. with stirring overnight. After cooling, the mixture is diluted with a saturated aqueous ammonium chloride solution, the aqueous phase is extracted three times with dichloromethane, the combined organic phases are dried over sodium sulfate and filtered, and the solvent is stripped off with gentle heating in vacuo. Column chromatography on silica gel results in 1.12 g (67% of theory) of product.

HPLC (Method 1): Rₜ=5.85 min

MS (ES+): m/z=507 (M+H)⁺.

¹H-NMR (400 MHz, CDCl₃): δ=7.14 (s, 1H), 4.92 (m, 1H), 4.11-4.31 (m, 2H), 3.78 (s, 3H), 2.74-2.97 (m, 3H), 1.95-2.12 (m, 3H), 1.59-1.75 (m, 6H), 1.47 (s, 9H), 1.24-1.45 (m, 3H), 1.15 (d, 3H), 0.90 (d, 3H), 0.80 (d, 3H), 0.53-0.77 (m, 2H).

### Example 58A

### Methyl 2-{isopropyl[(trans-4-methylcyclohexyl)carbonyl]amino}-5-piperidin-4-ylthiophene-3-carboxylate

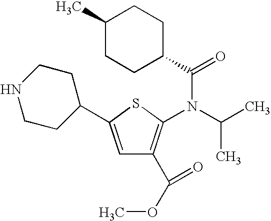

919.7 mg (1.27 mmol) of tert-butyl 4-[5-{isopropyl[(trans-4-methylcyclohexyl)carbonyl]amino}-4-(methoxycarbonyl)-2-thienyl]piperidine-1-carboxylate are dissolved in 7.5 ml of dichloromethane, and 7.5 ml of trifluoroacetic acid are added. The mixture is stirred at RT for 1 h. The mixture is then evaporated to dryness in vacuo with gentle heating, and the residue is dissolved in dichloromethane and washed with a saturated aqueous sodium bicarbonate solution. The aqueous phase is extracted three times with dichloromethane, the combined organic phases are dried over sodium sulfate, and the solvent is stripped off in vacuo with gentle heating. The residue is separated by flash chromatography on silica gel 60 to result in 528 mg (93% of theory) of product.

HPLC (Method 6): Rₜ=4.17 min

MS (ES+): m/z=407 (M+H)⁺.

¹H-NMR (400 MHz, CDCl₃): δ=8.36 (br. s, 1H), 7.20 (s, 1H), 4.86-4.98 (m, 1H), 3.79 (s, 3H), 3.49-3.59 (m, 2H), 2.96-3.08 (m, 3H), 1.97-2.30 (m, 4H), 1.54-1.76 (m, 4H), 1.22-1.49 (m, 3H), 1.15 (d, 3H), 0.89 (d, 3H), 0.80 (d, 3H), 0.56-0.77 (m, 2H).

### Example 59A

### Methyl 2-{isopropyl[(trans-4-methylcyclohexyl)carbonyl]amino}-5-[1-(methylsulfonyl)piperidin-4-yl]thiophene-3-carboxylate

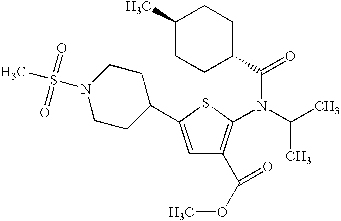

60.0 mg (0.15 mmol) of methyl 2-{isopropyl[(trans-4-methylcyclohexyl)carbonyl]amino}-5-piperidin-4-ylthiophene-3-carboxylate are dissolved in 1.0 ml of N,N′-dimethylformamide, and 44.8 mg (0.44 mmol) of triethylamine and 33.8 mg (0.30 mmol) of methanesulfonyl chloride are added. The reaction mixture is stirred at RT overnight. Without further workup, the reaction mixture is fractionated by preparative HPLC (RP-18 column, mobile phase: acetonitrile-water gradient) to result in 23 mg (28% of theory) of product.

HPLC S (Method 6): Rₜ=4.93 min

MS (ES+): m/z=485 (M+H)⁺.

¹H-NMR (400 MHz, DMSO-d₆): δ=7.21 (s, 1H), 4.69-4.80 (m, 1H), 3.72 (s, 3H), 3.58-3.69 (m, 2H), 2.76-3.04 (m, 5H), 1.93-2.15 (m, 3H), 1.40-1.73 (m, 7H), 1.13-1.38 (m, 3H), 1.09 (d, 3H), 0.81 (d, 3H), 0.77 (d, 3H), 0.46-0.71 (m, 2H).

### Example 60A

### Methyl 2-{isopropyl[(trans-4-methylcyclohexyl)carbonyl]amino}-5-{1-[(methylamino)carbonothioyl]piperidin-4-yl}thiophene-3-carboxylate

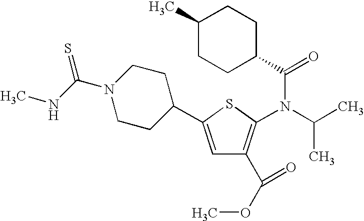

60.0 mg (0.15 mmol) of methyl 2-{isopropyl[(trans-4-methylcyclohexyl)carbonyl]amino}-5-piperidin-4-ylthiophene-3-carboxylate are dissolved in 1.0 ml of N,N′-dimethylformamide, and 44.8 mg (0.44 mmol) of triethylamine and 21.6 mg (0.30 mmol) of methyl isothiocyanate are added. The reaction mixture is stirred at RT overnight. Without further workup, the reaction mixture is fractionated by preparative HPLC (RP-18 column, mobile phase: acetonitrile-water gradient) to result in 37 mg (52% of theory) of product.

HPLC (Method 6): Rₜ=4.94 min

MS (DCI(NH₃)): m/z=481 (M+H)⁺.

¹H-NMR (400 MHz, DMSO-d₆): δ=7.64-7.75 (m, 1H), 7.17 (s, 1H), 4.61-4.82 (m, 3H), 3.71 (s, 3H), 2.96-3.21 (m, 3H), 2.91 (d, 3H), 1.90-2.09 (m, 3H), 1.38-1.68 (m, 7H), 1.15-1.34 (m, 2H), 1.07 (d, 3H), 0.81 (d, 3H), 0.76 (d, 3H), 0.46-0.71 (m, 2H).

### Example 61

### Methyl 2-amino-5-phenylthiophene-3-carboxylate

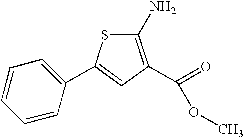

44.4 g (1.38 mol) of sulfur and 122 ml (1.38 mol) of methyl cyanoacetate are introduced into 280 ml of N,N-dimethylformamide. Then, at room temperature, 104 ml (0.747 mol) of triethylamine are added dropwise. 162 ml (1.38 mol) of phenylacetylaldehyde dissolved in 190 ml of N,N-dimethylformamide are slowly added dropwise to the reaction and then stirred at room temperature overnight. After the reaction is complete, the mixture is poured into water, whereupon a solid precipitates out. The suspension is then stirred for 3 h, the solid is filtered off and washed twice with water, and the solid is recrystallized from ethanol. 157 g of crystals (49% of theory) are obtained.

HPLC (Method 6): Rₜ=4.68 min

MS (DCI(NH3)): m/z=234 (M+H)⁺

¹H-NMR (400 MHz, DMSO-d₆) δ=7.51 (s, 2H), 7.45 (d, 2H), 6.35 (t, 2H), 7.25 (s, 1H), 7.18 (t, 1H), 3.74 (s, 3H).

### Example 62A

### tert-Butyl 2-amino-5-phenylthiophene-3-carboxylate

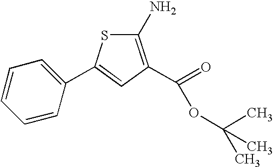

76.0 g (539 mmol) of tert-butyl cyanoacetate and 17.3 g (539 mmol) of sulfur are introduced into 200 ml of DMF under argon and, at RT, 29.4 g (291 mmol) of triethylamine are added. 71.9 g (539 mmol) of phenylacetaldehyde are added dropwise, and the mixture is stirred at RT overnight. The reaction mixture is then poured into water, stirred overnight, and the precipitate is filtered off with suction and washed with water, methanol and a little cyclohexane. Drying under high vacuum results in 89.3 g (57% of theory) of product.

HPLC (Method 1): Rₜ=4.92 min

MS (CI-pos): m/z=276 (M+H)⁺.

¹H-NMR (400 MHz, DMSO-d₆) δ=7.45 (d, 2H), 7.38 (s, 2H), 7.32 (t, 2H), 7.12-7.21 (m, 2H), 1.51 (s, 9H).

The following Examples 63A to 66A are prepared according to general procedure [A] from the appropriate starting compounds.

| Example | | Starting compounds | |
|---|---|---|---|
| No. | Structure | (yield) | Analytical data |
| 63A | 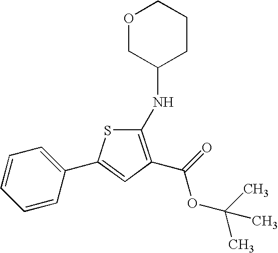 | 3.8 g (13.8 mmol) of aminothiophene of Example 62A; 2.8 g (27.7 mmol) of tetrahydropyran-3-one MS (ESIpos): (337 mg (7% of theory)) | HPLC (Method 6): Rₜ = 5.99 min MS (ESI-pos): m/z = 360 (M + H)⁺ |
| 64A | 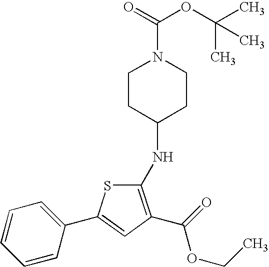 | 20.0 g (80.9 mmol) of aminothiophene of Example 1A; 64.5 g (323 mmol) of N-Boc-piperidin-4-one (note: double reaction of the amine with the ketone, repeat over 16 h, not 2 h) (16.5 g (47% of theory)) | HPLC (Method 6): Rₜ = 6.05 min MS (ESI-pos): m/z = 431 (M + H)⁺ |
| 65A | 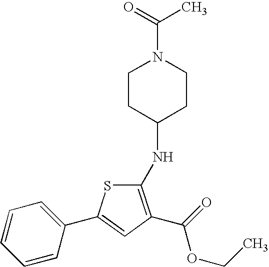 | 5.0 g (20.2 mmol) of aminothiophene of Example 1A; 11.4 g (80.9 mmol) of N-acetylpiperidin-4-one (note: double reaction of the amine with the ketone, repeat over 16 h, not 2 h) (3.5 g (44% of theory)) | HPLC (Method 6): Rₜ = 4.98 min MS (ESI-pos): m/z = 373 (M + H)⁺ |
| 66A | 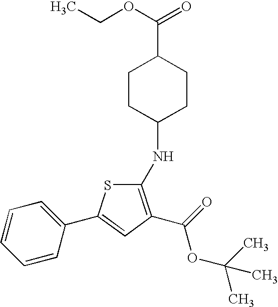 | 13.7 g (49.6 mmol) of aminothiophene of Example 62A; 16.9 g (99.1 mmol) of ethyl 4-cyclohexanonecarboxylate (17.0 g (80% of theory)) | HPLC (Method 6): Rₜ = 6.72 min MS (CI-pos): m/z = 430 (M + H)⁺ |

General Procedure [G]: Reductive Amination of 2-Aminothiophenes with Ketones in the Presence of Titanium Tetrachloride

21.4 mmol (2.0 equivalents) of the 2-aminothiophene and 10.7 mmol (1.0 equivalent) of the ketone are introduced into 30 ml of dichloroethane under argon. The reaction mixture is cooled to −78° C. (internal temperature about −70° C.) and then 11.8 mmol (1.1 equivalents) of a 1 molar solution of titanium tetrachloride in dichloromethane are added. The mixture is stirred at this temperature for 6-8 h and then 32.1 mmol (3.0 equivalents) of sodium triacetoxyborohydride are added. The mixture is stirred overnight, during which it slowly warms to room temperature. 1 ml of methanol is added (the mixture foams slightly to moderately, and a precipitate forms after a short time), and the mixture is stirred at room temperature for 30 min and subsequently added to a saturated sodium carbonate solution. The precipitate is removed by filtering the complete mixture through kieselguhr, the kieselgut is washed with copious dichloromethane, the organic phase is extracted with a saturated sodium bicarbonate solution, and the aqueous phase is washed with a saturated sodium chloride solution, separated off and dried over sodium sulfate. The desiccant is filtered off, and the solvent is removed in vacuo. Depending on the purity, the crude product can be reacted on directly or be purified by chromatography on silica gel with cyclohexane/ethyl acetate or dichloromethane/methanol mixtures.

### Example 67A

### Methyl 5-phenyl-2-(tetrahydro-2H-thiopyran-4-ylamino)thiophene-3-carboxylate

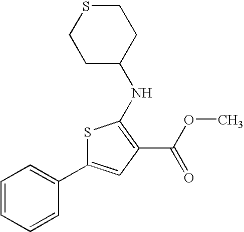

Starting with 2.50 g (10.7 mmol) of 2-aminothiophene from Example 61A and 2.49 g (21.4 mmol) of tetrahydrothiopyran-4-one, general procedure [G] results in 4.57 g (quant.) of crude product, dispensing with the filtration through kieselguhr. The crude product is reacted without further purification.

HPLC (Method 6): R=5.62 min

MS (CI-pos): m/z=334 (M+H)⁺

The following compound was synthesized in analogy to Example 97A:

| Example | | Starting compounds | |
|---|---|---|---|
| No. | Structure | (yield) | Analytical data |
| 68A | 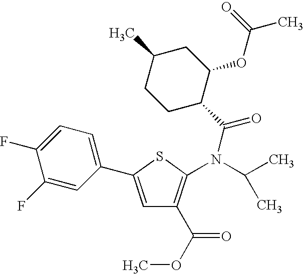 | 282 mg (0.63 mmol) Example 95A, (48 mg (15% of theory)) | HPLC (Method 1): Rₜ = 5.69 min MS (DCI): m/z = 494 (M + H)⁺ |

The following Examples 69A to 72A are prepared according to general procedure [G] from the appropriate starting compounds.

| Example | | Starting compounds | |
|---|---|---|---|
| No. | Structure | (yield) | Analytical data |
| 69A | 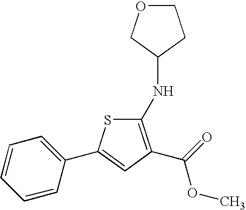 | 761 mg (3.08 mmol) of aminothiophene of Example 1A; 530 mg (616 mmol) of tetrahydrofuran-3-one (1.02 g (quant.)) | HPLC (Method 6): Rₜ = 5.29 min MS (ESI-pos): m/z = 318 (M + H)⁺ |
| 70A | 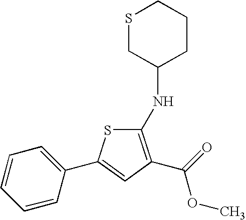 | 2.5 g (10.7 mmol) of aminothiophene of Example 61A; 2.2 g (21.4 mmol) of tetrahydrothiphen-4-one (2.49 g (70% of theory)) | HPLC (Method 1): Rₜ = 5.71 min MS (CI-pos): m/z = 334 (M + H)⁺ |
| 71A | 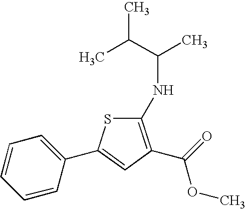 | 2.0 g (8.57 mmol) of aminothiophene of Example 61A; 1.48 g (17.1 mmol) of 3-methylbutan-2-one (2.34 g (59% of theory)) | HPLC (Method 6): Rₜ = 6.27 min MS (ESI-pos): m/z = 304 (M + H)⁺ |
| 72A | 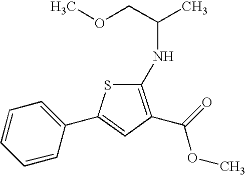 | 2.0 g (8.57 mmol) of aminothiophene of Example 61A; 1.5 g (17.1 mmol) of 2-methoxyacetone (2.58 g (91% of theory)) | HPLC (Method 1): Rₜ = 5.48 min MS (ESI-pos): m/z = 306 (M + H)⁺ |

### Example 73A

### Ethyl 2-[[(trans-4-methylcyclohexyl)carbonyl](tetrahydro-2H-pyran-4-yl)amino]-5-phenylthiophene-3-carboxylate

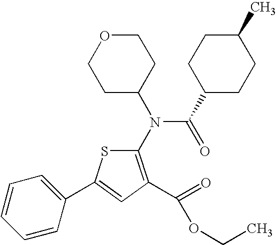

1.09 g (6.8 mmol) of the acid chloride and 1.75 g (13.6 mmol) of Hünig's base are added to 750 mg (2.3 mmol) of the amine from Example 23A, and the mixture is stirred at 80° C. After 16 h, there is scarcely is any conversion according to an HPLC check, and therefore a further 1.09 g (6.8 mmol) of the acid chloride are added, and the mixture is again stirred at 80° C. overnight. The next day, 1.09 g (6.8 mmol) of the acid chloride are again added, and the mixture is stirred at 80° C. for a further 2 days. The mixture is then mixed with ethyl acetate and extracted three times with a saturated sodium bicarbonate solution, the organic phase is dried over sodium sulfate and filtered, and the solvent is removed in vacuo. The product was purified in portions by preparative HPLC (Method 2). Yield: 288 mg (28% of theory).

HPLC (Method 6): Rₜ=5.93 min

MS (CI-pos): m/z=456 (M+H)⁺

The following Examples 74A to 83A are prepared according to general procedure [B] (see table for variant) from the appropriate starting compounds.

| Example | | Starting compounds | | |
|---|---|---|---|---|
| No. | Structure | (yield) | Variant | Analytical data |
| 74A | 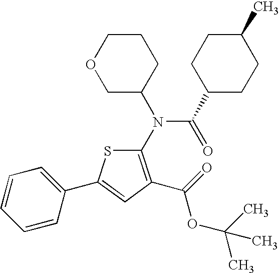 | 150 mg (0.42 mmol) of the amine from Example 63A, 0.5 ml of acid chloride 150 mg (74% of theory) | 2 | HPLC (Method 6): Rₜ = 6.51 min MS (ESI-pos): m/z = 484 (M + H)⁺ |
| 75A | 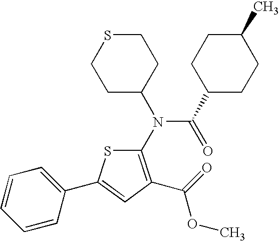 | 1.32 g (3.0 mmol) of the amine from Example 67A, twice 1.44 g of acid chloride 913 mg (63% of theory) | 2 | HPLC (Method 6): Rₜ = 6.14 min MS (CI-pos): m/z = 458 (M + H)⁺ |
| 76A | 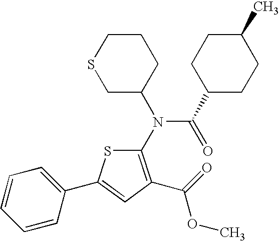 | 300 mg (0.9 mmol) of the amine from Example 70A, 0.3 ml of acid chloride 243 mg (59% of theory) | 2 | HPLC (Method 6): Rₜ = 6.24 min MS (CI-pos): m/z = 458 (M + H)⁺ |
| 77A | 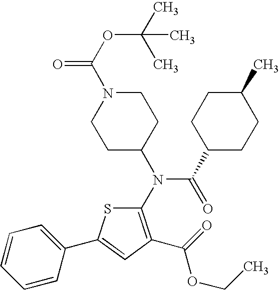 | 5.31 g (12.3 mmol) of the amine from Example 64A, 5.94 g (37 mmol) of acid chloride 6.0 g (22% of theory) | 1 | HPLC (Method 6): Rₜ = 6.37 min MS (CI-pos): m/z = 579 (M + NH₄)⁺ |
| 78A | 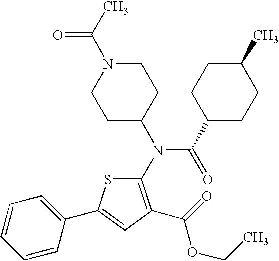 | 500 mg (1.34 mmol) of the amine from Example 65A, 648 mg (4.0 mmol) of acid chloride 167 mg (25% of theory) | 1 | HPLC (Method 6): Rₜ = 5.39 min MS (CI-pos): m/z = 497 (M + H)⁺ |
| 79A | 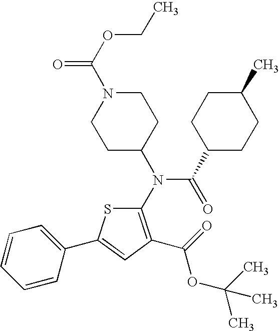 | 200 mg (0.47 mmol) of the amine from Example 66A, 0.5 ml of acid chloride, 181 mg (1.4 mmol) of Hünig's base 120 mg (46% of theory) | 2 | HPLC (Method 6): Rₜ = 6.99 min MS (ESI-pos): m/z = 554 (M + H)⁺ |
| 80A | 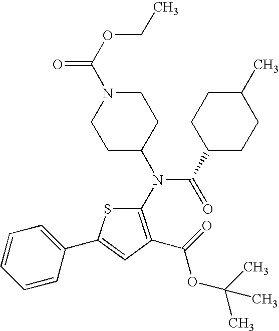 | 4.4 g (10.2 minol) of the amine from Example 66A, 4.75 g (30.7 mmol) of acid chloride 4.6 g (81% of theory) | 1 | HPLC (Method 6): Rₜ = 6.11 min MS (ESI-pos): m/z = 548 (M + H)⁺ |
| 81A | 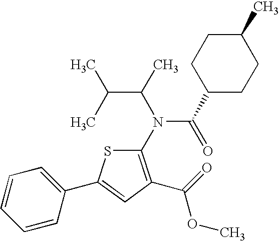 | 307 mg (0.66 mmol) of the amine from Example 71A, 318 mg (1.98 mmol) of acid chloride 4.6 g (81% of theory) | 3 | HPLC (Method 6): Rₜ = 6.63 min MS (ESI-pos): m/z = 428 (M + H)⁺ |
| 82A | 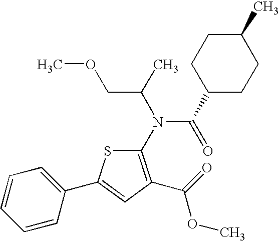 | 215 mg (0.66 mmol) of the amine from Example 72A, 316 mg (1.98 mmol) of acid chloride 146 mg (52% of theory) | 3 | HPLC (Method 6): Rₜ = 5.90 min MS (CI-pos): m/z = 430 (M + H)⁺ |
| 83A | 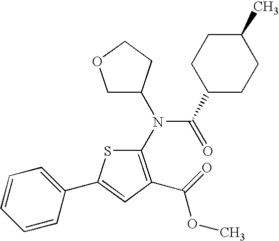 | 150 mg (0.47 mmol) of the amine from Example 69A; 0.5 ml of acid chloride (120 mg (55% of theory)) | 2 | HPLC (Method 6): Rₜ = 5.83 min MS (CI-pos): m/z = 442 (M + H)⁺ |

### Example 84A

### Ethyl 2-[[(trans-4-methylcyclohexyl)carbonyl](piperidin-4-yl)amino]-5-phenylthiophene-3-carboxylate

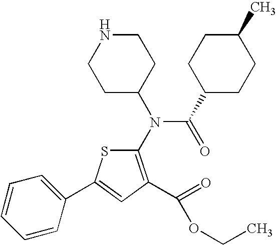

360 mg (0.62 mmol) of the amide from Example 77A are dissolved in 28 ml of dioxane, 7 ml of a 1N hydrochloric acid solution are added, and the mixture is stirred at 100° C. for 3 days. Since conversion is only very slow, a further 3.5 ml of the 1N hydrochloric acid are added. Stirring is continued at 100° C. for a further 3 days, and the starting compound has reacted. There is formation not only of the product (44% of theory) but also of the carboxylic acid (52% of theory) produced by hydrolysis of the ethyl ester. The mixture is neutralized with a 1N lithium hydroxide solution, the solvent is removed in vacuo, and the crude product is employed for the complete ester hydrolysis.

HPLC (Method 3): Rₜ=2.27 min

MS (ESI-pos): m/z=455 (M+H)⁺

### Example 85A

### Ethyl 2-([(trans-4-methylcyclohexyl)carbonyl]{1-[(trans-4-methylcyclohexyl)carbonyl]piperidin-4-yl}amino)-5-phenylthiophene-3-carboxylate

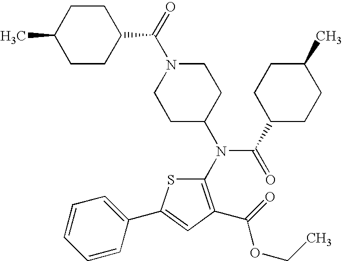

5.3 g (12.3 mmol) of the amine from Example 64A are dissolved in 60 ml of pyridine, 1.98 g (12.3 mmol) of the acid chloride are added, and the mixture is stirred at 120° C. overnight. Since conversion is only very small, 1.98 g (12.3 mmol) of acid chloride are again added, and the mixture is again stirred at 120° C. overnight. The mixture is then diluted with ethyl acetate and the organic phase is extracted three times each with 1N hydrochloric acid and a saturated sodium bicarbonate solution. The organic phase is dried over sodium sulfate and filtered, and the solvent is removed in vacuo. The product is purified by chromatography on silica gel with cyclohexane-ethyl acetate mixtures. 1.05 g (14% of theory) of product are obtained.

HPLC (Method 6): Rₜ=5.25 min

MS (ESI-pos): m/z=520 (M+H)⁺

### Example 86A

### 4-[[3-(tert-Butoxycarbonyl)-5-phenyl-2-thienyl](4-methylbenzoyl)amino]cyclohexanecarboxylic acid

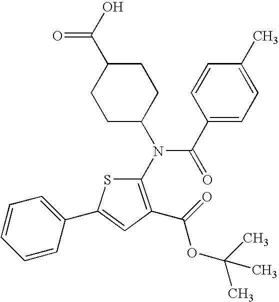

7.77 g (13.0 mmol) of the ester from Example 80A are dissolved in 80 ml of dioxane, 19.6 ml (19.6 mmol) of a 1N solution of lithium hydroxide in water are added, and the mixture is stirred at 40° C. overnight. Since the conversion is not yet complete, a further 10.9 ml of the lithium hydroxide solution are added, and the mixture is again stirred at 40° C. overnight. The mixture is concentrated on a rotary evaporator, the residue is taken up in water, and the pH is adjusted to 4 with 1N hydrochloric acid, whereupon the product precipitates. The solid is filtered off, washed with water and dried under high vacuum overnight. 6.74 g (94% of theory) of product are obtained.

HPLC (Method 6): Rₜ=5.25 min

MS (ESI-pos): m/z=520 (M+H)⁺

### Example 87A

### Methyl 2-[[(trans-4-methylcyclohexyl)carbonyl](piperidin-4-yl)amino]-5-phenylthiophene-3-carboxylate

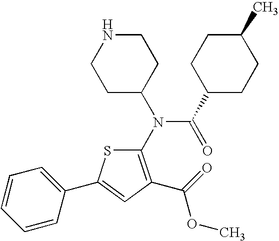

2.73 g (6.4 mmol) of the acid from Example 43 are introduced into 20 ml of methanol, the mixture is cooled to 0° C., and then 0.84 g (7.0 mmol) of thionyl chloride are slowly added dropwise. The mixture is stirred overnight, during which it slowly warms to room temperature. Since an HPLC check indicates scarcely any conversion, the mixture is heated to 40° C. and stirred at this temperature overnight. The HPLC check indicates about 50% conversion, whereupon the volatile components are removed in vacuo, the residue is taken up in 20 ml of methanol and, at room temperature, 1 ml of thionyl chloride is added. After the mixture has been stirred under reflux overnight, all the volatile components are again removed in vacuo and the residue is codistilled with 30 ml of methanol. LC-MS analysis of the crude product indicates a content of 88% the ester and 5% of the starting material. The crude product is reacted without further purification.

HPLC (Method 3): Rₜ=2.16 min

MS (ESI-pos): m/z=441 (M+H)⁺

### Example 88A

### Methyl 2-[[(trans-4-methylcyclohexyl)carbonyl](piperidin-4-yl)amino]-5-phenylthiophene-3-carboxylate

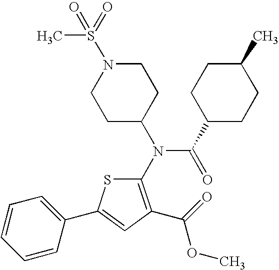

100 mg (0.23 mmol) of the amine from Example 87A are dissolved in 2 ml of tetrahydrofuran, 39 mg (0.34 mmol) of methanesulfonyl chloride and 44 mg (0.34 mmol) of Hünig's base are added, and the reaction mixture is stirred at room temperature overnight. The solvent is then removed in vacuo and the product is purified by preparative HPLC (Method 2). 22 mg (19% of theory) of product are obtained.

HPLC (Method 6): Rₜ=5.56 min

MS (ESI-pos): m/z=519 (M+H)⁺

General Procedure [H]: Reaction of Aliphatic Carboxylic Acids with Amines in the Presence of PyBOP

1.0 equivalent of carboxylic acid, 1.05 equivalents of amine and 1.1 equivalents of PyBOP are introduced into tetrahydrofuran under argon (0.05-0.1 M solution based on the carboxylic acid), 1.1 equivalents of Hünig's base are added, and the mixture is stirred at room temperature overnight. The solvent is then removed in vacuo, and the product is purified by preparative HPLC or chromatography on silica gel with cyclohexane/ethyl acetate mixtures.

### Example 89A

### tert-Butyl 2-([(trans-4-methylcyclohexyl)carbonyl]{4-[(4-methylpiperazin-1-yl)carbonyl]cyclohexyl}amino)-5-phenylthiophene-3-carboxylate

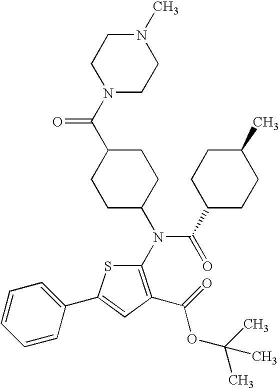

Starting from 150 mg (0.29 mmol) of the acid from Example 86A and 30 mg (0.30 mmol) of N-methylpiperazine, 190 mg (96% of theory) of product are obtained after purification by preparative HPLC (Method 2).

HPLC (Method 6): Rₜ=4.85 min

MS (ESI-pos): m/z=609 (M+H)⁺

The following Examples 90A to 92A are prepared according to general procedure [H] from the appropriate starting compounds:

| Example | | Starting compounds | |
|---|---|---|---|
| No. | Structure | (yield) | Analytical data |
| 90A | 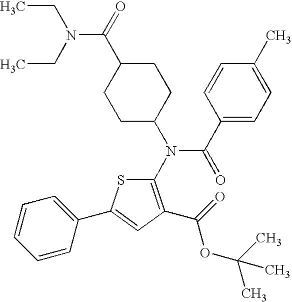 | 150 mg (0.29 mmol) of acid from Example 86A, 22 mg (0.30 mmol) of diethylamine (57 mg (34% of theory)) | HPLC (Method 1): Rₜ = 5.75 min; MS (ESI-pos): m/z = 519 (M + H)⁺ |
| 91A | 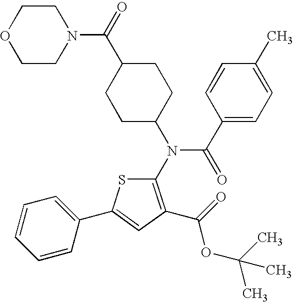 | 150 mg (0.29 mmol) of acid from Example 86A, 26 mg (0.30 mmol) of morpholine (135 mg (79% of theory)) | HPLC (Method 6): Rₜ = 5.39 min; MS (ESI-pos): m/z = 589 (M + H)⁺ |
| 92A | 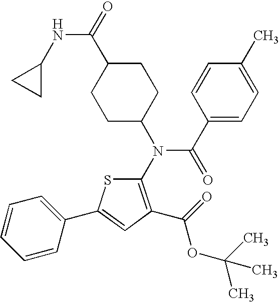 | 150 mg (0.29 mmol) of acid from Example 86A, 17 mg (0.30 mmol) of cyclopropylamine (81 mg (50% of theory)) | HPLC (Method 6): Rₜ = 5.34 min; MS (ESI-pos): m/z = 559 (M + H)⁺ |

### Example 93A

### Methyl 2-({[(1 r,2s,4r)-2-(acetyloxy)-4-methylcyclohexyl]carbonyl}amino)-5-phenylthiophene-3-carboxylate (racemate)

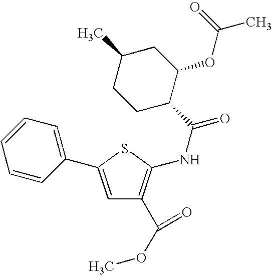

Racemic (1r,2s,4r)-2-(acetyloxy)-4-methylcyclohexanecarboxylic acid is synthesized from racemic ethyl 4-methyl-2-oxocyclohexanecarboxylate in analogy to WO 02/100851. 266 mg (1.33 mmol) of (1r,2s,4r)-2-(acetyloxy)-4-methylcyclohexanecarboxylic acid are dissolved in 3 ml of thionyl chloride and stirred at room temperature for 1 h. The reaction mixture is evaporated to dryness and taken up in 2 ml of dioxane, and 281.6 mg (1.21 mmol) of methyl 2-amino-5-phenylthiophene-3-carboxylate are added. The solution is stirred under reflux for 2 h. The cooled reaction mixture is partitioned between ethyl acetate and a saturated sodium bicarbonate solution. The combined organic phases are dried over magnesium sulfate and evaporated in vacuo. Purification of the residue on silica gel 60 by MPLC (mobile phase: cyclohexane/ethyl acetate 5/1) results in 404.3 mg (80% of theory) of the product.

HPLC (Method 6): Rₜ=5.60 min

MS (DCI(NH3)): m/z=433 (M+NH₄)⁺

¹H-NMR (400 MHz, DMSO-d₆): δ=11.06 (s, 1H), 7.64 (d, 2H), 7.55 (s, 1H), 7.41 (t, 2H), 7.40 (t, 1H), 5.34 (s, 1H), 3.87 (s, 3H), 2.86-2.93 (m, 1H), 1.94 (s, 3H), 1.76-1.90 (m, 4H), 1.60-1.72 (m, 1H), 1.27 (t, 1H), 1.00-1.13 (m, 1H), 0.88 (d, 3H).

In analogy to the synthesis of Example 93A, enantiopure (1R,2S,4R)-2-(acetyloxy)-4-methylcyclohexanecarboxylic acids are prepared (in analogy to WO 04/052885) and converted to the following compounds:

| Example | | Starting compounds | |
|---|---|---|---|
| No. | Structure | (yield) | Analytical data |
| 94A | 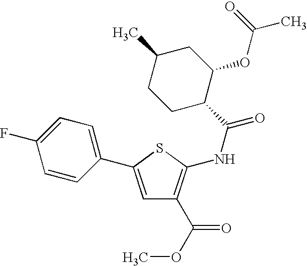 | 120 mg (0.48 mmol) of Example 45A, 112 mg (0.51 mmol) of (1R,2S,4R)-2-(acetyloxy)-4- methylcyclohexanecarboxylic acid (139 mg (67% of theory)) | HPLC (Method 6): Rₜ = 5.73 min; MS (ESI-pos): m/z = 434 (M + H)⁺ |
| 95A | 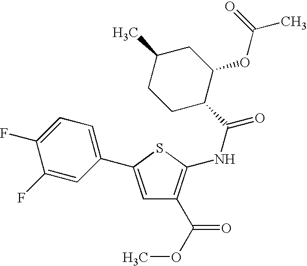 | 208 mg (0.77 mmol) of Example 46A, 166 mg (0.83 mmol) of (1R,2S,4R)-2-(acetyloxy)-4- methylcyclohexanecarboxylic acid (304 mg (87% of theory)) | HPLC (Method 6): Rₜ = 5.67 min; MS (DCI): m/z = 469 (M + NH₄)⁺ |
| 96A | 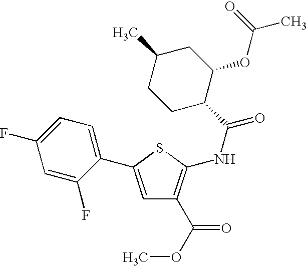 | 155 mg (0.58 mmol) of Example 47A, 124 mg (0.62 mmol) of (1R,2S,4R)-2-(acetyloxy)-4- methylcyclohexanecarboxylic acid (246 mg (84% of theory)) | HPLC (Method 6): Rₜ = 5.66 min; MS (DCI): m/z = 469 (M + NH₄)⁺ |

### Example 97A

### Methyl 2-[{[(1 r,2s,4r)-2-(acetyloxy)-4-methylcyclohexyl]carbonyl}(isopropyl)amino]-5-phenylthiophene-3-carboxylate (racemate)

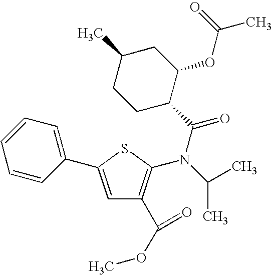

398 mg (0.96 mmol) of the compound from Example 93A, 759 mg (2.87 mmol) of 18-crown-6, 2.65 g (19.1 mmol) of potassium carbonate are introduced into 15 ml of N,N-dimethylformamide and then 3.25 g (1.91 ml, 19.1 mmol) of 2-iodopropane are added. The reaction mixture is stirred at 40° C. overnight. The cooled reaction mixture is partitioned between ethyl acetate and water, the organic phase is dried over magnesium sulfate, and the solvent is evaporated off in vacuo. This crude product is reacted twice more in analogy to the previous procedure. After the third reaction, the residue is purified on silica gel 60 by MPLC (mobile phase: cyclohexane/ethyl acetate 4/1). 404 mg (21% of theory) of product are obtained.

HPLC (Method 6): Rₜ=5.55 min

MS (ESI+): m/z=458 (M+H)⁺

¹H-NMR (400 MHz, DMSO-d₆): δ=7.81 (s, 1H), 7.75 (d, 2H), 7.45 (t, 2H), 7.38 (t, 1H), 5.25 (s, 1H), 4.65-4.81 (m, 1H), 3.75 (s, 3H), 1.85-2.03 (m, 4H), 1.54-1.71 (m, 3H), 1.40-1.50 (m, 1H), 0.70-1.30 (m, 12H, including: 1.11 (d, 3H), 0.80 (d, 3H), 0.74 (d, 3H)).

The following compound was synthesized in analogy to the synthesis of Example 97A:

| Example | | Starting compounds | |
|---|---|---|---|
| No. | Structure | (yield) | Analytical data |
| 98A | 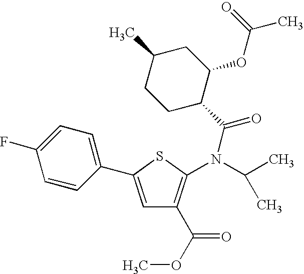 | 122 mg (0.48 mmol) of Example 94A, (17 mg (11% of theory)) | HPLC (Method 4): Rₜ = 3.23 min; MS (ESI-pos): m/z = 476 (M + H)⁺ |

### Example 99A

### Methyl 2-{[1-methyl-2-(methylthio)ethyl]amino}-5-phenylthiophene-3-carboxylate

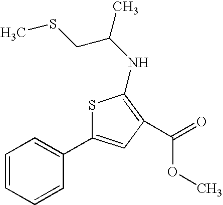

2.0 g (8.56 mmol) of the amine from Example 61A and 1.79 g (17.15 mmol) of 1-methylthiopropan-2-one are dissolved in 20 ml of dichloromethane under argon, the solution is cooled to −78° C., and 9.4 ml (9.43 mmol) of a 1N solution of titanium tetrachloride in dichloromethane are added. The mixture is stirred at this temperature for 7.5 h and then 5.45 g (25.72 mmol) of sodium triacetoxyborohydride are added, and the reaction mixture is stirred overnight, during which it warms to room temperature. 1 ml of methanol is then cautiously added to the mixture (a precipitate precipitates out), and the mixture is stirred for a further 30 min and then poured into a saturated sodium bicarbonate solution. The aqueous phases are extracted three times with dichloromethane. The combined organic phases are extracted once with a saturated sodium bicarbonate solution and once with a saturated sodium chloride solution, dried over sodium sulfate and filtered, and the solvent is removed in vacuo. Yield: 2.86 g (92% of theory). The product is reacted without further purification.

HPLC (Method 6): Rₜ=5.64 min

MS (ESI-pos): m/z=322 (M+H)⁺

### Example 100A

### Methyl 2-{[(trans-4-methylcyclohexyl)carbonyl][1-methyl-2-(methylthio)ethyl]amino}-5-phenylthiophene-3-carboxylate

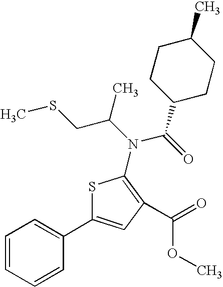

500 mg (1.56 mmol) of the amine from Example 99A are mixed with 0.5 ml of trans-4-methylcyclohexanecarbonyl chloride, and the mixture is stirred in a closed vessel at 90° C. for 48 h. The mixture is then taken up with ethyl acetate, the organic phase is extracted three times with a saturated sodium bicarbonate solution, dried over sodium sulfate and filtered, and the solvent is removed in vacuo. The product is purified by preparative HPLC. Yield: 456 mg (66% of theory).

HPLC (Method 1): Rₜ=5.17 min

MS (ESI-pos): m/z=446 (M+H)⁺

### Example 101A

### Methyl 2-{[(trans-4-methylcyclohexyl)carbonyl][1-methyl-2-(methylsulfinyl)ethyl]amino}-5-phenylthiophene-3-carboxylate

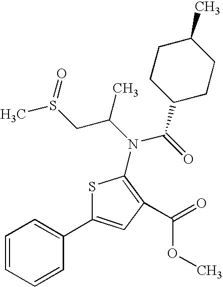

225 mg (0.51 mmol) of the thioeter from Example 100A are introduced into 5 ml of dichloromethane, the solution is cooled to −78° C., and 125 mg (0.51 mmol) of meta-chloroperbenzoic acid (70-75% in water) are added in portions (suspension). After 5 h at −78° C., the mixture is diluted with dichloromethane and added to a saturated thiosulfate solution. The organic phase is separated, extracted once with a saturated sodium bicarbonate solution, dried over sodium sulfate and filtered, and the solvent is removed in vacuo. The product is purified by preparative HPLC. Yield: 183 mg (79% of theory).

HPLC (Method 1): Rₜ=5.03 min

MS (ESI-pos): m/z=462 (M+H)⁺

Exemplary Embodiments

### Example 1

### 2-[Isopropyl-(trans-4-methylcyclohexanecarbonyl)amino]-5-phenylthiophene-3-carboxylic acid

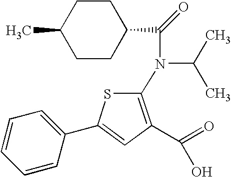

5.00 g (12.1 mmol) of ethyl 2-[isopropyl-(trans-4-methyl-cyclohexanecarbonyl)amino]-5-phenylthiophene-3-carboxylate are dissolved in 40 ml of dioxane under argon. 40 ml of a 1N lithium hydroxide solution are added, and the mixture is stirred at 100° C. for 4 h. It is then concentrated, dichloromethane and water are added, the pH is adjusted to 5 with hydrochloric acid, the phases are separated, the aqueous phase is extracted twice with dichloromethane, and the combined organic phases are dried over sodium sulfate, filtered and concentrated. Purification by recrystallization from dichloromethane/acetonitrile results in 3.10 g (67% of theory) of product. Alternatively, purification is also possible by preparative HPLC (RP-18 column, mobile phase: acetonitrile-water gradient 95:5→5:95).

LC-MS (Method 5): Rₜ=2.85 min

MS (ESIpos): m/z=386 (M+H)⁺.

¹H-NMR (300 MHz, DMSO-d₆): δ=13.1 (s, 1H), 7.78-7.70 (m, 3H), 7.49-7.34 (m, 3H), 4.79 (sept, 1H), 4.31-4.16 (m, 2H), 2.22-2.09 (m, 1H), 1.73-1.44 (m, 5H), 1.37-1.19 (m, 2H), 1.16 (d, 3H), 0.92 (d, 3H), 0.75 (d, 3H), 0.74-0.53 (m, 2H).

Examples 2 to 10 listed in the following table are prepared from the appropriate starting compounds in analogy to the method of Example 1.

| | | Prepared in | |
|---|---|---|---|
| | | analogy to | |
| Example | | Example No. | |
| No. | Structure | (yield) | Analytical data |
| 2 | 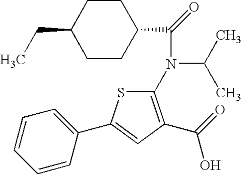 | 1 from Example 8A (72% of theory) | LC-MS (Method 5): Rₜ = 3.00 min MS (ESIpos): m/z = 400 (M + H)⁺ |
| 3 | 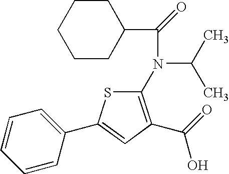 | 1 from Example 9A (75% of theory) | LC-MS (Method 5): Rₜ = 2.68 min MS (ESIpos): m/z = 372 (M + H)⁺ |
| 4 | 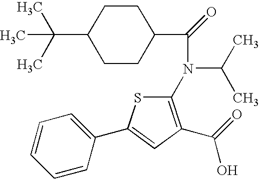 | 1 from Example 10A (92% of theory) | LC-MS (Method 5): Rₜ = 3.13 min MS (ESIpos): m/z = 428 (M + H)⁺ |
| 5 | 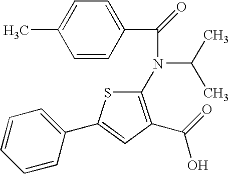 | 1 from Example 15A (71% of theory) | LC-MS (Method 5): Rₜ = 2.50 min MS (ESIpos): m/z = 380 (M + H)⁺ |
| 6 | 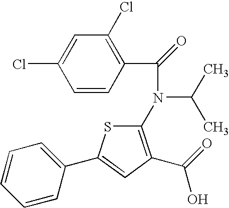 | 1 from Example 7A (95% of theory) | LC-MS (Method 5): Rₜ = 2.72 min MS (ESIpos): m/z = 434 (M + H)⁺ |
| 7 | 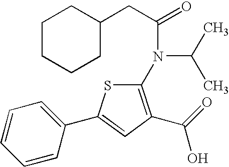 | 1 from Example 11A (88% of theory) | LC-MS (Method 5): Rₜ = 2.90 min MS (ESIpos): m/z = 386 (M + H)⁺ |
| 8 | 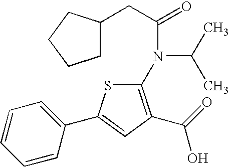 | 1 from Example 12A (88% of theory) | LC-MS (Method 5): Rₜ = 2.76 min MS (ESIpos): m/z = 372 (M + H)⁺ |
| 9 | 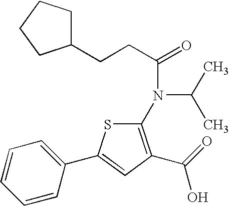 | 1 from Example 13A (76% of theory) | LC-MS (Method 4): Rₜ = 3.05 min MS (ESIpos): m/z = 386 (M + H)⁺ |
| 10 | 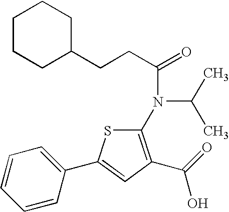 | 1 from Example 14A (76% of theory) | LC-MS (Method 4): Rₜ = 3.17 min MS (ESIpos): m/z = 400 (M + H)⁺ |

General Procedure [C]: Ester Hydrolysis

0.37 mmol (1.0 equivalent) of the ester are dissolved in 10 ml of dioxane, and 0.75 ml (4.0 equivalents) of a 1N solution of lithium hydroxide in water is added. The solution is stirred at 100° C. for 16 h and then adjusted to pH 7 with 1N hydrochloric acid, and the solvent is removed in vacuo. The product is purified by preparative HPLC (Method 2) or by chromatography on silica gel.

### Example 11

### 2-[(4-Methylbenzoyl)(cyclohexyl)amino]-5-phenylthiophenecarboxylic acid

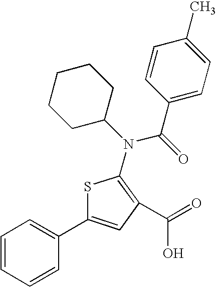

Starting from 110 mg (0.25 mmol) of ester from Example 22A, general procedure [C] and preparative HPLC (Method 2) result in 51 mg (50% of theory) of product.

HPLC (Method 1): Rₜ=5.54 min

MS (ESI-pos): m/z=420 (M+H)⁺

The Examples 12 to 17 are prepared in an analogous manner according to general procedure [C] from the appropriate starting compounds.

| | | | | HPLC |
|---|---|---|---|---|
| Example | | Ester | | Rₜ [min] |
| No. | Structure | [amount of ester] | Yield | (Method) |
| 12 | 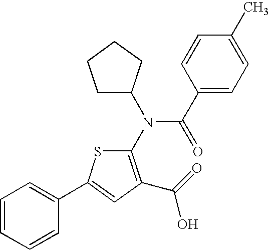 | Example 21A 120 mg (0.28 mmol) | 51 mg (45% of theory) | 5.33 (Method 1) |
| 13 | 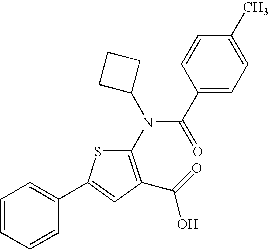 | Example 20A 120 mg (0.29 mmol) | 3 mg (3% of theory) | 5.13 (Method 5) |
| 14 | 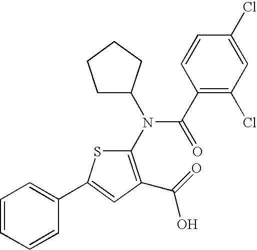 | Example 19A 88 mg (0.18 mmol) | 59 mg (71% of theory) | 5.50 (Method 1) |
| 15 | 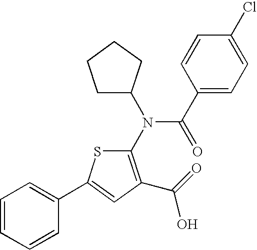 | Example 18A 166 mg (0.37 mmol) | 52 mg (33% of theory) | 5.28 (Method 1) |
| 16 | 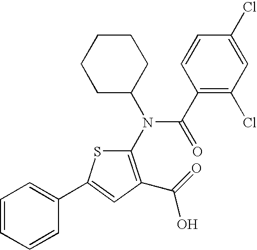 | Example 17A 74 mg (0.15 mmol) | 48 mg (69% of theory) | 5.67 (Method 1) |
| 17 | 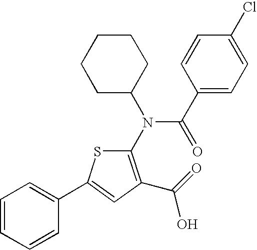 | Example 16A 87 mg (0.19 mmol) | 55 mg (67% of theory) | 5.45 (Method 1) |
| 18 | 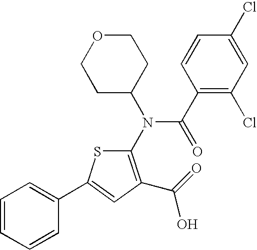 | Example 24A 85 mg (0.17 mmol) | 66 mg (81% of theory) | 5.08 (Method 6) |

### Example 19

### 5-(4-Fluorophenyl)-2-{isopropyl[(trans-4-methylcyclohexyl)carbonyl]amino}thiophene-3-carboxylic acid

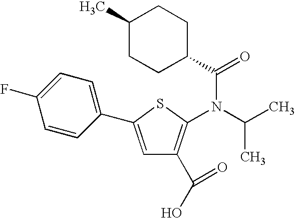

153.0 mg (0.37 mmol) of the compound from Example 28A are dissolved in 3 ml of dioxane, and 1.5 ml of an aqueous 1N solution of lithium hydroxide are added. The mixture is stirred at RT overnight, acidified with a 1N hydrochloric acid solution and extracted with ethyl acetate. The organic phase is dried over sodium sulfate, and the solvent is stripped off in vacuo with gentle heating. 137 mg (93% of theory) of product are obtained.

HPLC (Method 6): Rₜ=5.44 min

MS (ESI+): m/z=404 (M+H)⁺.

¹H-NMR (400 MHz, CDCl₃): δ=7.52-7.67 (m, 3H), 7.08-7.19 (m, 2H), 4.89-5.09 (m, 1H), 2.13-2.27 (m, 1H), 1.56-1.79 (m, 5H), 1.28-1.54 (m, 2H), 1.25 (d, 3H), 1.00 (d, 3H), 0.79 (d, 3H), 0.60-0.75 (m, 2H).

### Example 20

### 5-(2,4-Difluorophenyl)-2-{isopropyl[(trans-4-methylcyclohexyl)carbonyl]amino thiophene-3-carboxylic acid

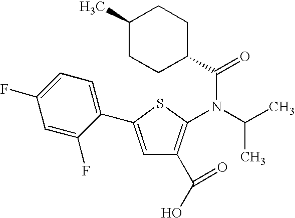

96.0 mg (0.22 mmol) of the compound from Example 29A are dissolved in 2 ml of dioxane, and 2 ml of an aqueous 1N lithium hydroxide solution are added. The mixture is stirred at RT for 2 h, acidified with a 1N hydrochloric acid solution and purified by preparative HPLC (RP-18 column, mobile phase: acetonitrile-water gradient). 81 mg (87% of theory) of product are obtained.

HPLC (Method 6): Rₜ=5.44 min

MS (ES+): m/z=422 (M+H)⁺.

¹H-NMR (400 MHz, DMSO-d₆): δ=13.17 (br. s, 1H), 7.89-8.01 (m, 1H), 7.80 (s, 1H), 7.39-7.51 (m, 1H), 7.15-7.26 (m, 1H), 4.72-4.88 (m, 1H), 2.04-2.18 (m, 1H), 1.40-1.71 (m, 5H), 1.18-1.35 (m, 2H), 1.14 (d, 3H), 0.90 (d, 3H), 0.75 (m, 3H), 0.51-0.72 (m, 2H).

### Example 21

### 5-(3,4-Difluorolphenyl)-2-{isopropyl[(trans-4-methylcyclohexyl)-carbonyl]amino}thiophene-3-carboxylic acid

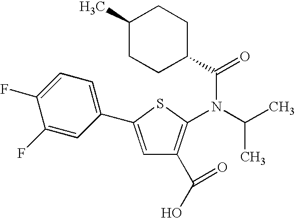

150.0 mg (0.37 mmol) of methyl 5-bromo-2-{isopropyl[(trans-4-methylcyclohexyl)carbonyl]amino}thiophene-3-carboxylate from Example 27A are dissolved in 2 ml of N,N′-dimethylformamide under argon, and 176.6 mg (1.12 mmol) of 3,4-difluorophenylboronic acid and 820 μof an aqueous 2N sodium carbonate solution are added. Argon is passed through the reaction solution at 80° C. for 1 h. Then 30.4 mg (0.04 mmol) of bis[(diphenylphosphino)ferrocene]palladium(II) chloride are added as catalyst, and the mixture is stirred at 80° C. for 20 h. After cooling, the reaction solution is filtered and purified by preparative HPLC (RP-18 column, mobile phase: acetonitrile-water gradient). 62 mg (39% of theory) of product and 51 mg of the product protected as methyl ester are obtained.

HPLC (Method 1): Rₜ=5.48 min

MS (ES+): m/z=422 (M+H)⁺.

¹H-NMR (400 MHz, CDCl₃): δ=7.64 (s, 1H), 7.38-7.47 (m, 1H), 7.30-7.37 (m, 1H), 7.18-7.28 (m, 1H), 4.92-5.06 (m, 1H), 2.12-2.25 (m, 1H), 1.18-1.79 (m, 10H), 1.0 (d, 3H), 0.79 (d, 3H), 0.61-0.77 (m, 2H).

### Example 22

### 2-{Isopropyl[(trans-4-methylcyclohexyl)carbonyl]amino}-5-(1,3-thiazol-2-yl)thiophene-3-carboxylic acid

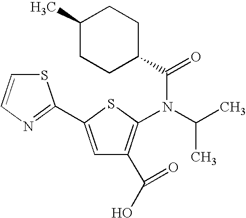

73.0 mg (0.18 mmol) of the compound from Example 38A are dissolved in 2 ml of dioxane, and 2 ml of an aqueous 1N lithium hydroxide solution are added. The mixture is stirred at RT overnight and then dioxane is removed in vacuo with gentle heating. The residue is acidified with a 1N hydrochloric acid solution and extracted with ethyl acetate. The organic phase is dried over sodium sulfate, and the solvent is stripped off in vacuo with gentle warming. 58 mg (82% of theory) of product are obtained without further purification.

HPLC (Method 1): Rₜ=4.80 min

MS (DCI(NH₃)): m/z=393 (M+H)⁺.

¹H-NMR (400 MHz, DMSO-d₆): δ=13.3 (br. s, 1H), 7.82-7.89 (m, 3H), 4.72-4.88 (m, 1H), 2.04-2.20 (m, 1H), 1.39-1.72 (m, 5H), 1.18-1.37 (m, 2H), 1.15 (d, 3H), 0.90 (d, 3H), 0.75 (d, 3H), 0.51-0.72 (m, 2H).

Examples 23 to 30 listed in the following table are prepared from the appropriate starting compounds based on the method of Example 22. Solvent volume, amount of 1N of lithium hydroxide solution, reaction time and reaction temperature is varied where appropriate.

| | | | [Amount of | |
|---|---|---|---|---|
| | | | dioxane] | |
| | | | [amount of | |
| | | Ester | 1N LiOH] | Analytical data |
| Ex. | | [amount of ester] | Time | HPLC (Method) |
| No. | Structure | (yield) | Temp. | MS (Method) |
| 23 | 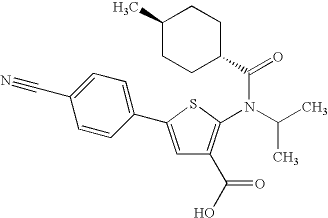 | Example 30A 68 mg (0.16 mmol) 37 mg (56% of theory) | 3.0 ml 1.5 ml 18 h RT | HPLC (Method 6): Rₜ = 5.08 min MS (ES+): m/z = 411 (M + H)⁺ |
| 24 | 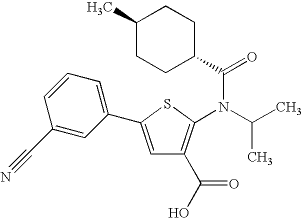 | Example 31A 81 mg (0.19 mmol) 62 mg (79% of theory) | 3.0 ml 1.5 ml 18 h RT | HPLC (Method 6): Rₜ = 5.10 min MS (ES+): m/z = 411 (M + H)⁺ |
| 25 | 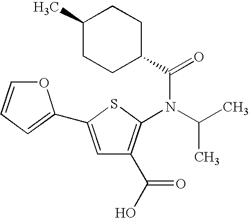 | Example 32A 85 mg (0.17 mmol) 39 mg (58% of theory) | 1.0 ml 1.0 ml 2 h RT | HPLC (Method 6): Rₜ = 5.02 min MS (DCI(NH₃)): m/z = 376 (M + H)⁺ |
| 26 | 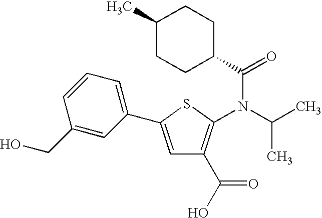 | Example 33A 84 mg (0.20 mmol) 38 mg (47% of theory) | 1.0 ml 0.5 ml 18 h RT | HPLC (Method 6): Rₜ = 4.63 min MS (ES+): m/z = 416 (M + H)⁺ |
| 27 | 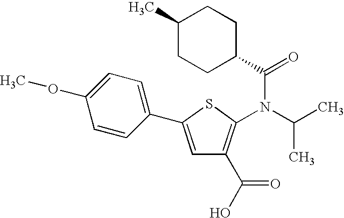 | Example 34A 83 mg (0.19 mmol) 63 mg (78% of theory) | 3.0 ml 1.5 ml 18 h RT | HPLC (Method 6): Rₜ = 5.35 min MS (ES+): m/z = 416 (M + H)⁺ |
| 28 | 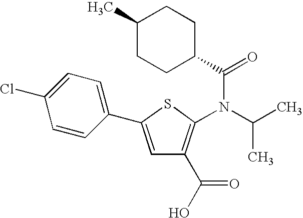 | Example 35A 75 mg (0.17 mmol) 45 mg (62% of theory) | 1.0 ml 0.5 ml 18 h RT | HPLC (Method 6): Rₜ = 5.80 min MS (ES+): m/z = 420 (M + H)⁺ |
| 29 | 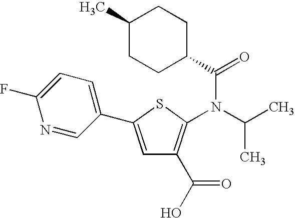 | Example 36A 49 mg (0.12 mmol) 45 mg (95% of theory) | 1.0 ml 1.0 ml 2 h RT | HPLC (Method 6): Rₜ = 4.92 min MS (ES+): m/z = 405 (M + H)⁺ |
| 30 | 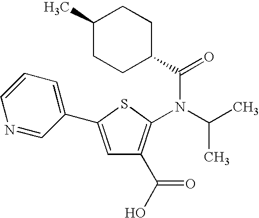 | Example 37A 18.6 mg (0.05 mmol) 18.mg (quant.) | 1.0 ml 0.2 ml overnight RT | HPLC (Method 6): Rₜ = 4.02 min MS (DCI(NH₃)): m/z = 387 (M + H)⁺ |

### Example 31

### 2-{[(trans-4-Methylcyclohexyl)carbonyl][1-methyl-2-(methylthio)-ethyl]amino}-5-phenylthiophene-3-carboxylic acid

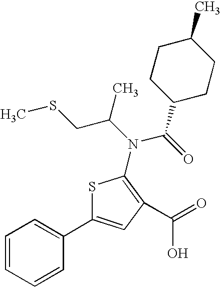

200 mg (0.45 mmol) of the methyl ester from Example 100A are introduced into 3 ml of dioxane and 0.3 ml of water, 48 mg (2.02 mmol) of lithium hydroxide are added, and the mixture is stirred under reflux for 5 h. The mixture is then neutralized with 2 ml of 1N hydrochloric acid, the solvent is removed in vacuo, and the product is purified by preparative

HPLC. Yield: 165 mg (85% of theory).

HPLC (Method 6): Rₜ=5.58 min

MS (CI-pos): m/z=432 (M+H)⁺

¹H-NMR (400 MHz, CDCl₃): δ [ppm]=7.70-7.60 (m, 3H); 7.46-7.34 (m, 3H); 5.18-5.11 (m, 1H); 2.73-2.59 (m, 2H); 2.20 (s, 3H); 1.91-1.86 (m, 0.5H); 1.73-1.60 (m, 4.5H); 1.54-1.25 (m, 4H); 1.10 (d, 2H); 0.79 (d, 3H); 0.75-0.66 (m, 2H).

### Example 32

### 5-(4-Fluorophenyl)-2-[[(trans-4-methylcyclohexyl)carbonyl]-(tetrahydro-2H-pyran-4-yl)amino]-thiophene-3-carboxylic acid

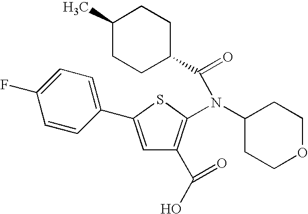

100.8 mg (0.22 mmol) of methyl 5-(4-fluorophenyl)-2-[[(trans-4-methylcyclohexyl)carbonyl](tetrahydro-2H-pyran-4-yl)amino]thiophene-3-carboxylate are dissolved in 1.5 ml of dioxane and 1.5 ml of an aqueous 1N lithium hydroxide solution are added. The mixture is stirred at RT overnight, acidified with a 1N hydrochloric acid solution and extracted with ethyl acetate. The organic phase is dried over sodium sulfate and the solvent is stripped off in vacuo with gentle heating. The residue is prepurified by preparative HPLC (RP-18 column, mobile phase: acetonitrile-water gradient), and the cis/trans mixture is fractionated by preparative HPLC (Zorbax SB C-18 column, mobile phase: 35:65 0.2% trifluoroacetic acid:acetonitrile). 48 mg (49% of theory) of product are obtained.

HPLC (Method 6): Rₜ=5.11 min

MS (ESI+): m/z=446 (M+H)⁺.

¹H-NMR (400 MHz, CDCl₃): δ=7.54-7.64 (m, 3H), 7.09-7.18 (m, 2H), 4.76-4.92 (m, 1H), 3.85-4.05 (m, 2H), 3.41-3.59 (m, 2H), 2.11-2.25 (m, 1H), 1.57-1.92 (m, 8H), 1.23-1.54 (m, 3H), 0.60-0.85 (m, 5H).

### Example 33

### 5-(3,4-Difluorophenyl)-2-[[(trans-4-methylcyclohexyl)carbonyl]-(tetrahydro-2H-pyran-4-yl)amino]thiophene-3-carboxylic acid

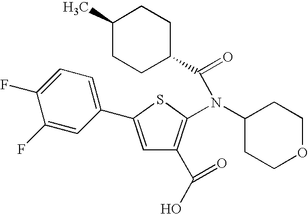

163.4 mg (0.34 mmol) of methyl 5-(3,4-difluorophenyl)-2-[[(trans-4-methylcyclohexyl)carbonyl](tetrahydro-2H-pyran-4-yl)amino]thiophene-3-carboxylate are dissolved in 1.5 ml of dioxane and 1.5 ml of an aqueous 1N lithium hydroxide solution are added. The mixture is stirred at RT overnight, acidified with a 1N hydrochloric acid solution and extracted with ethyl acetate. The organic phase is dried over sodium sulfate and the solvent is stripped off in vacuo with gentle heating. The residue is prepurified by preparative HPLC (RP-18 column, mobile phase: acetonitrile-water gradient), and the cis/trans mixture is fractionated by preparative HPLC (Zorbax SB C-18 column, mobile phase: 35:65 0.2% trifluoroacetic acid:acetonitrile). 26 mg (16% of theory) of product are obtained.

HPLC (Method 6): Rₜ=5.17 min

MS (ESI+): m/z=464 (M+H)⁺.

¹H-NMR (400 MHz, CDCl₃): δ=7.62 (s, 1H), 7.38-7.47 (m, 1H), 7.30-7.37 (m, 1H), 7.19-7.30 (m, 1H), 4.77-4.92 (m, 1H), 3.86-4.06 (m, 2H), 3.40-3.59 (m, 2H), 2.10-2.23 (m, 1H), 1.56-1.91 (m, 8H), 1.21-1.54 (m, 3H), 0.59-0.83 (m, 5H).

### Example 34

### 5-(2,4-Difluorophenyl)-2-[[(trans-4-methylcyclohexyl)carbonyl]-(tetrahydro-2H-pyran-4-yl)amino]thiophene-3-carboxylic acid

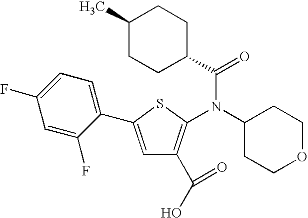

69.3 mg (0.15 mmol) of methyl 5-(2,4-difluorophenyl)-2-[[(trans-4-methylcyclohexyl)carbonyl](tetrahydro-2H-pyran-4-yl)amino]thiophene-3-carboxylate are dissolved in 1.5 ml of dioxane and 1.5 ml of an aqueous 1N lithium hydroxide solution are added. The mixture is stirred at RT overnight, acidified with a 1N hydrochloric acid solution and extracted with ethyl acetate. The organic phase is dried over sodium sulfate and the solvent is stripped off in vacuo with gentle heating. The residue is prepurified by preparative HPLC (RP-18 column, mobile phase: acetonitrile-water gradient), and the cis/trans mixture is fractionated by preparative HPLC (Zorbax SB C-18 column, mobile phase: 35:65 0.2% trifluoroacetic acid:acetonitrile). 34 mg (51% of theory) of product are obtained.

HPLC (Method 6): Rₜ=5.14 min

MS (ESI+): m/z=464 (M+H)⁺.

¹H-NMR (400 MHz, CDCl₃): δ=7.77 (s, 1H), 7.57-7.67 (m, 1H), 6.92-7.04(m, 2H), 4.77-4.94 (m, 1H), 3.89-4.09 (m, 2H), 3.42-3.64 (m, 2H), 2.04-2.25 (m, 1H), 1.57-1.95 (m, 8H), 1.21-1.55 (m, 3H), 0.59-0.85 (m, 5H).

### Example 35

### 2-{Isopropyl[(trans-4-methylcyclohexyl)carbonyl]amino}-5-[1-(methylsulfonyl)piperidin-4-yl]thiophene-3-carboxylic acid

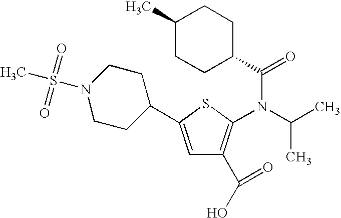

21.0 mg (0.04 mmol) of methyl 2-{isopropyl[(trans-4-methylcyclohexyl)carbonyl]amino}-5-[1-(methylsulfonyl)piperidin-4-yl]thiophene-3-carboxylate are dissolved in 1.0 ml of dioxane, and 0.5 ml of an aqueous 1N lithium hydroxide solution are added. The mixture is stirred at RT overnight, and then the solvent is removed in vacuo with gentle heating. The residue is acidified with a 1N hydrochloric acid solution and extracted with ethyl acetate. The organic phase is dried over Extrelut® and the solvent is stripped off in vacuo with gentle heating. The residue is purified by preparative HPLC (RP-18 column, mobile phase: acetonitrile-water gradient). 12 mg (59% of theory) of product are obtained.

HPLC (Method 6): Rₜ=4.49 min

MS (ES+): m/z=471 (M+H)⁺.

¹H-NMR (400 MHz, CDCl₃): δ=7.22 (s, 1H), 4.83-5.02 (m, 1H), 3.84-3.97 (m, 2H), 2.74-3.00 (m, 6H), 2.11-2.21 (m, 2H), 1.97-2.11 (m, 1H), 1.76-1.93 (m, 2H), 1.54-1.73 (m, 5H), 1.23-1.50 (m, 2H), 1.18 (d, 3H), 0.93 (d, 3H), 0.79 (d, 3H), 0.51-0.76 (m, 2H).

### Example 36

### 2-}Isopropyl[(trans-4-methylcyclohexyl)carbonyl]amino}-5-{1-[(methylamino)carbonothioyl]-piperidin-4-yl}thioiphen-3-carboxylic acid

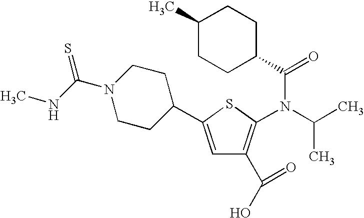

33.0 mg (0.07 mmol) of methyl 2-{isopropyl[(trans-4-methylcyclohexyl)carbonyl]amino}-5-{1-[(methylamino)carbonothioyl]piperidin-4-yl}thiophene-3-carboxylate are dissolved in 1.0 ml of dioxane, and 0.5 ml of an aqueous 1N lithium hydroxide solution are added. The mixture is stirred at RT overnight and then acidified with a 1N hydrochloric acid solution and extracted with ethyl acetate. The organic phase is dried over sodium sulfate and the solvent is stripped off in vacuo with gentle heating. 32 mg (94% of theory) of product are obtained without further purification steps.

HPLC (Method 6): Rₜ=4.51 min

MS (ES+): m/z=466 (M+H)⁺.

¹H-NMR (400 MHz, DMSO-d₆): δ=7.64-7.74 (m, 1H), 7.14 (s, 1H), 4.61-4.81 (m, 3H), 2.96-3.20 (m, 3H), 2.91 (d, 3H), 1.93-2.09 (m, 3H), 1.37-1.69 (m, 6H), 1.14-1.34 (m, 3H), 1.08 (d, 3H), 0.84 (d, 3H), 0.76 (d, 3H), 0.45-0.71 (m, 2H).

General Procedure [I]: Cleavage of Tert-Butyl Esters with Trifluoroacetic Acid

The tert-butyl ester to be cleaved is taken up in a mixture of dichloromethane and trifluoroacetic acid, and the reaction mixture is stirred at room temperature until a check of conversion (HPLC) indicates complete conversion, typically 30-60 min. The solvent mixture is removed in vacuo, and the crude product is purified by preparative HPLC (Method 2).

### Example 37

### 2-([(trans-4-Methylcyclohexyl)carbonyl]{-4-[(4-methylpiperazin-1-yl)carbonyl]cyclohexyl}amino)-5-phenylthiophene-3-carboxylic acid

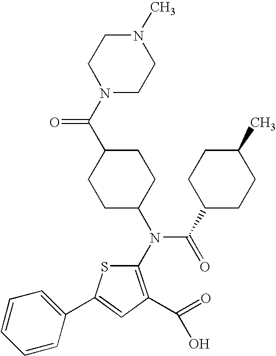

Preparation takes place according to general procedure [I] from the compound from Example 89A.

HPLC (Method 6): Rₜ=4.61 min

MS (ESI-pos): m/z=551 (M+H)⁺

¹H-NMR (400 MHz, DMSO-d₆): δ [ppm]=7.75-7.71 (m, 3H); 7.46 (t, 2H); 7.38 (t, 1H); 4.45-4.35 (m, 1H); 2.82 (s, 1H); 2.25 (s, 2H); 2.20-2.10 (m, 3H; overlapped by H₂O signal); 1.9-1.2 (m, 16H); 0.74 (d, 3H); 0.69-0.55 (m, 2H).

The following Examples 38 to 41 are prepared according to general procedure [I] from the appropriate starting compound:

| Example | | Starting compound | |
|---|---|---|---|
| No. | Structure | (yield) | Analytical data |
| 38 | 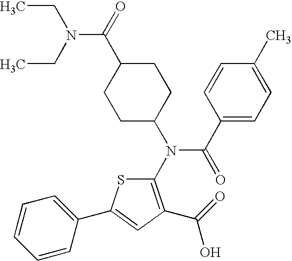 | 57 mg (0.1 mmol) of the tert-butyl ester from Example 90A (23 mg (44% of theory)) | HPLC (Method 6): Rₜ = 4.95 min; MS (ESI-pos): m/z = 519 (M + H)⁺ |
| 39 | 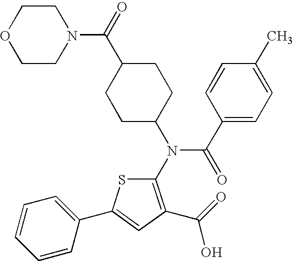 | 118 mg (0.2 mmol) of the tert-butyl ester from Example 91A (88 mg (82% of theory)) | HPLC (Method 6): Rₜ = 4.62 min; MS (ESI-pos): m/z = 533 (M + H)⁺ |
| 40 | 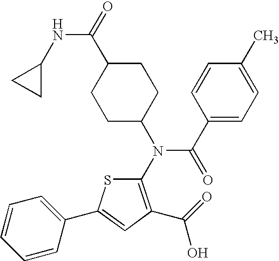 | 60 mg (0.11 mmol) of the tert-butyl ester from Example 92A (48 mg (89% of theory)) | HPLC (Method 6): Rₜ = 4.55 min; MS (ESI-pos): m/z = 503 (M + H)⁺ |
| 41 | 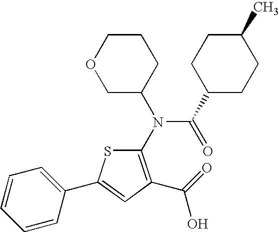 | 148 mg (0.31 mmol) of the tert-butyl ester from Example 74A (68 mg (52% of theory)) | HPLC (Method 1): Rₜ = 5.26 min; MS (CI-pos): m/z = 428 (M + H)⁺ |

General Procedure [J]: Ester Hydrolysis

0.37 mmol (1.0 equivalent) of the ester are dissolved in dioxane, and 1-10 equivalents of lithium hydroxide are added. This can take place either in the form of an aqueous solution, for example 1N, or as solid. In the latter case, water is also added to the dioxane, typically in a ratio of dioxane:water=10:1 to 20:1. The solution is stirred at 100° C. for 2-16 h and then adjusted to pH 7 with 1N hydrochloric acid, and the solvent is removed in vacuo. The product is purified by preparative HPLC (Method 2) or by chromatography on silica gel.

### Example 42

### 2-[[(trans-4-Methylcyclohexyl)carbonyl](tetrahydro-2H-pyran-4-yl)amino]-5-phenylthiophene-3-carboxylic acid

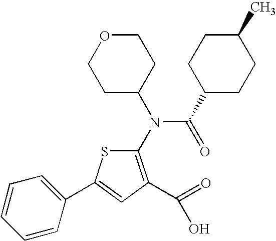

206 mg (0.45 mmol) of the ester from Example 73A are dissolved in 5 ml of dioxane, and 0.68 ml (0.68 mmol) of a 1N solution of lithium hydroxide in water are added. The reaction mixture is stirred at 100° C. for 16 h and then the solvent is removed in vacuo, and the product is purified by preparative HPLC (Method 2). 52 mg (27% of theory) of product are obtained.

HPLC (Method 6): Rₜ=5.21 min

MS (ESI-pos): m/z=428 (M+H)⁺

¹H-NMR (400 MHz, DMSO-d₆): δ [ppm]=7.62 (d, 2H), 7.53 (s, 1H); 7.41 (t, 2H); 7.29 (t, 1H); 4.55-4.45 (m, 1H); 3.88-3.82 (m, 1H); 3.80-3.75 (m, 1H); 3.38-3.28 (m, 2H; overlapped by H₂O signal); 2.37-2.26 (m, 1H); 1.89 (d, 1H); 1.78 (d, 1H); 1.70-1.50 (m, 5H); 1.42-1.15 (m, 4H); 0.74 (d, 3H); 0.69-0.53 (m, 2H).

### Example 43

### 2-[[(trans-4-Methylcyclohexyl)carbonyl](piperidin-4-yl)amino]-5-phenylthiophene-3-carboxylic acid

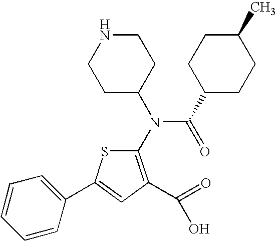

390 mg (0.86 mmol) of the ester from Example 85A are dissolved in 20 ml of dioxane, and 1.3 ml (1.3 mmol) of a 1N lithium hydroxide solution in water are added. The reaction mixture is stirred at 100° C. overnight and then adjusted to pH=7 with 1N hydrochloric acid, the solvent is removed in vacuo and the product is purified by preparative HPLC (Method 2). 93 mg (23% of theory) of product are obtained.

HPLC (Method 6): Rₜ=4.53 min

MS (ESI-pos): m/z=427 (M+H)⁺

¹H-NMR (400 MHz, DMSO-d₆): δ [ppm]=13.28 (s, 1H); 8.74 (s_{b}, 1H); 8.18 (s_{b}, 1H); 7.82 (s, 1H); 7.74 (d, 2H); 7.47 (t, 2H); 7.40 (t, 1H); 4.75-4.67 (m, 1H); 3.35-3.24 (m, signal overlapped by H₂O signal); 3.06-3.00 (m, 2H); 2.20-2.12 (m, 1H); 1.93-1.25 (m, 1H); 0.76 (d, 3H); 0.70-0.58 (m, 1H).

The following Examples 44 to 49 are prepared according to general procedure [J] from the appropriate starting compounds:

| Example | | Starting compounds | | |
|---|---|---|---|---|
| No. | Structure | (yield) | Conditions | Analytical data |
| 44 | 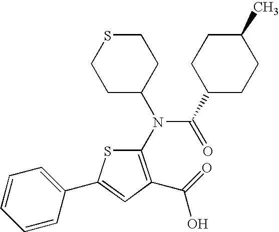 | 81 mg (0.18 mmol) of the ester from Example 75A (70 mg (89% of theory)) | 19 mg (0.8 mmol) of LiOH, dioxane:H₂O =10:1, 100° C., 3 h | HPLC (Method 1): Rₜ = 6.46 min; MS (ESI-pos): m/z = 484 (M + H)⁺ |
| 45 | 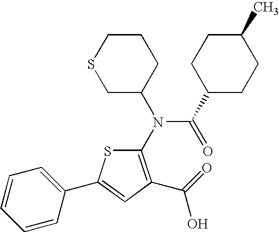 | 225 mg (0.49 mmol) of the ester from Example 76A (176 mg (81% of theory)) | 53 mg (2.21 mmol) of LiOH, dioxane:H₂O =10:1, 100° C., 6 h | HPLC (Method 6): Rₜ = 5.66 min; MS (ESI-pos): m/z = 444 (M + H)⁺ |
| 46 | 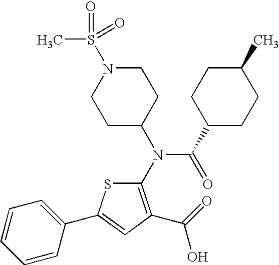 | 38 mg (0.07 mmol) of the ester from Example 88A (26 mg (70% of theory)) | 6 mg (0.26 mmol) of LiOH, dioxane:H₂O =20:1, 100° C., 3.5 h | HPLC (Method 6): Rₜ = 5.08 min; MS (ESI-pos): m/z = 505 (M + H)⁺ |
| 47 | 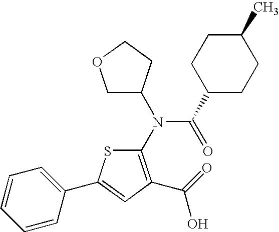 | 117 mg (0.27 mmol) of the ester from Example 83A (15 mg (14% of theory)) | 1.2 mg (1.19 mmol) of 1N LiOH solution 100° C., 16 h | HPLC (Method 6): Rₜ = 5.08 min; MS (ESI-pos): m/z = 414 (M + H)⁺ |
| 48 | 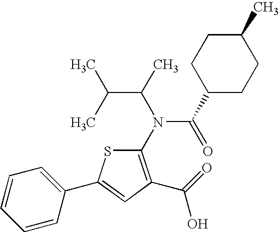 | 93 mg (0.22 mmol) of the ester from Example 81A (75 mg (83% of theory)) | 23 mg (0.98 mmol) of LiOH, dioxane:H₂O =10:1, 100° C., 10 h | HPLC (Method 6): Rₜ = 5.92 min; MS (ESI-pos): m/z = 414 (M + H)⁺ |
| 49 | 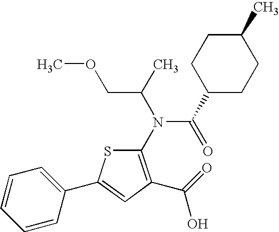 | 126 mg (0.29 mmol) of the ester from Example 82A (106 mg (87% of theory)) | 32 mg (1.32 mmol) of LiOH, dioxane:H₂O =10:1, 100° C., 10 h | HPLC (Method 6): Rₜ = 5.33 min; MS (ESI-pos): m/z = 416 (M + H)⁺ |

General Procedure [K]: Reductive Amination of N-Piperidinyl Derivatives with Aldehydes and Ketones

1.0 equivalent of the piperidine is introduced into 1,2-dichloroethane under argon, and 2.0 equivalents of the carbonyl compound are added. If the amine is in the form of the hydrochloride, 1.0 equivalent of Hünig's base is added. The reaction mixture is stirred at room temperature for 2 h and then 2.0 equivalents of sodium triacetoxyborohydride and 4.0 equivalents of acetic acid are added, and the reaction mixture is stirred at 40° C. overnight. A saturated sodium bicarbonate solution is then cautiously added, and the mixture is extracted three times with dichloromethane. The combined organic phases are dried over sodium sulfate and filtered, and the solvent is removed in vacuo. If larger amounts of starting material are still present, the reaction can optionally be repeated. The product is purified by preparative HPLC or chromatography on silica gel with cyclohexane/ethyl acetate mixtures.

### Example 50

### 2-{(1-Isopropylpiperidin-4-yl)[(trans-4-methylcyclohexyl)carbonyl]amino}-5-phenylthiophene-3-carboxylic acid

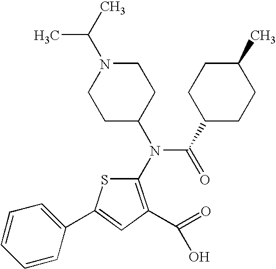

Starting with 73 mg (0.16 mmol) of piperidine from Example 43 and 18 mg (0.32 mmol) of acetone and reacting the mixture twice according to general procedure [K] results in 29 mg (39% of theory) of product. A difference from the general procedure [K] is that the product precipitates when the crude product is taken up in acetonitrile and is filtered off with suction, stirred with water, filtered off with suction, stirred with dichloromethane, filtered off with suction and then dried under high vacuum.

HPLC (Method 6): Rₜ=4.63 min

MS (ESI-pos): m/z=469 (M+H)⁺

¹H-NMR (400 MHz, DMSO-d₆): δ [ppm]=7.61 (d, 2H); 7.52 (s, 1H); 7.40 (t, 2H); 7.29 (t, 1H); 4.30-4.15 (m, 1H); 2.77 (d, 1H); 2.69 (d, 1H); 2.63-2.56 (m, 1H); 2.36-2.30 (m, 1H); 2.14 (t, 1H); 2.06 (t, 1H); 1.89 (d, 1H); 1.79 (d, 1H); 1.70-1.62 (m, 2H); 1.56-1.45 (m, 3H); 1.40-1.31 (m, 1H); 1.30-1.05 (m, 2H); 0.90, 0.89 (d, 6H); 0.74 (d, 3H); 0.68-0.53 (m, 2H).

### Example 51

### 2-{[(trans-4-Methylcyclohexyl)carbonyl][1-(tetrahydro-2H-pyran-3-yl)piperidin-4-yl]amino}-5-phenylthiophene-3-carboxylic acid

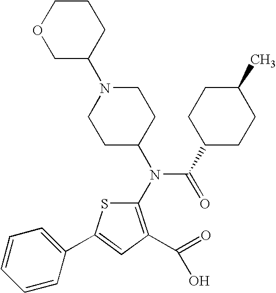

Starting with 60 mg (0.14 mmol) of piperidine from Example 43 and 28 mg (0.28 mmol) of tetrahydropyran-3-one, reacting the mixture twice according to general procedure [K] results in 12 mg (17% of theory) of product.

HPLC (Method 6): Rₜ=4.59 min

MS (ESI-pos): m/z=511 (M+H)⁺

¹H-NMR (400 MHz, DMSO-d₆): δ [ppm]=7.75-7.72 (m, 3H); 7.45 (t, 2H); 7.38 (t, 1H); 4.39-4.32 (m, 1H); 3.78 (d, 1H); 3.68 (d, 1H); 3.15-3.06 (m, 2H); 2.92-2.83 (m, 2H); 2.33-2.13 (m, 4H); 1.86-1.04 (m, 15H); 0.75 (d, 3H); 0.69-0.59 (m, 2H).

### Example 52

### 2-[{[(1 r,2s, 4r)-2-Hydroxy-4-methylcyclohexyl]carbonyl}(isopropyl)amino]-5-phenylthiophene-3-carboxylic acid (racemate)

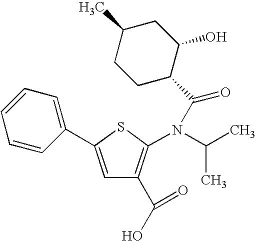

78.8 mg (0.17 mmol) of the compound from Example 97A are dissolved in 3 ml of a dioxane/water 2/1 mixture, then 430 μl (0.86 mmol) of a 2N lithium hydroxide solution are added, and the reaction is stirred at room temperature overnight. The solution is mixed with ethyl acetate and washed successively with 2N hydrochloric acid and water. The organic phase is dried over magnesium sulfate and then the solvent is evaporated off in vacuo. The obtained crude product is stirred with a petroleum ether/diethyl ether mixture, filtered and dried. 52 mg (70% of theory) of product are obtained.

HPLC (Method 6): Rₜ=5.06 min

MS (ESI+): m/z=402 (M+H)⁺

¹H-NMR (300 MHz, DMSO-d₆) δ=13.18 (br. s, 1H), 7.71-7.83 (m, 3H), 7.34-7.51 (m, 3H), 4.76-4.91 (m, 1.5H), 4.25 (d, 0.5H), 4.00 (s, 0.5H), 3.90 (s, 0.5H), 2.21-2.40 (m, 1H), 1.33-1.94 (m, 5H), 1.11-1.24 (m, 3H), 0.50-1.97 (m, 8H).

### Example 53 and Example 54

The racemate of the compound from Example 52 is separated into the enantiomers by preparative HPLC on a chiral phase (column: chiral silica gel selector KBD 5326; 10 μm; 250 mm×30 mm, based on the selector poly(N-methacryloyl-L-leucine dicyclopropylmethylamide), eluent: ethyl acetate, flow rate: 30 ml/min, UV detection: 254 nm, temperature: 24° C., sample loaded in ethyl acetate).

19 mg of enantiomer 1 (Example 53) and 17 mg of enantiomer 2 (Example 54) are obtained from 52 mg of racemate.

### Example 53

### 2-[{[(1S,2R, 4S)-2-Hydroxy-4-methylcyclohexyl]carbonyl}(isopropyl)amino]-5-phenylthiophen-3-carboxylic acid

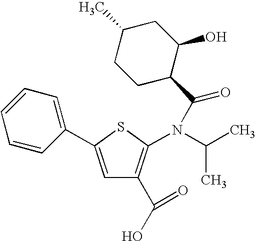

Retention time on chiral phase: Rₜ=11.3 min.

HPLC (Method 6): Rₜ=5.06 min.

### Example 54

### 2-[{[(1R, 2S,4R)-2-Hydroxy-4-methylcyclohexyl]carbonyl}(isopropyl)amino]-5-phenylthiophene-3-carboxylic acid

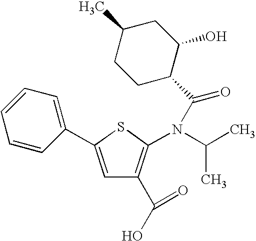

Retention time on chiral phase: Rₜ=14.0 min.

HPLC (Method 6): Rₜ=5.06 min.

MS (ESI+): m/z=402 (M+H)⁺

¹H-NMR (300 MHz, CDCl₃) δ=7.71 (s, 1H), 7.62 (d, 2H), 7.32-7.50 (m, 3H), 4.90-5.10 (m, 1H), 4.20 (br s, 0.7H), 3.97 (br s, 0.3H), 2.31 (d, 1H), 1.75-2.10 (m, 3H), 1.50-1.73 (m, 2H), 1.28 (d, 3H), 0.60-1.10 (m, 8H, including 1.01 [d, 3H], 0.79 [d, 3H]).

The enantiopure synthesis of Example 54 is carried out in analogy to the synthesis of the racemic material starting with (1R, 2S,4R)-2-(acetyloxy)-4-methylcyclohexanecarboxylic acid. See WO 04/052885 for the synthesis of (1R, 2S,4R)-2-(acetyloxy)-4-methylcyclohexanecarboxylic acid.

The following examples are prepared enantiopure in analogy to Example 54:

| Example | | Starting compounds | | |
|---|---|---|---|---|
| No. | Structure | (yield) | Conditions | Analytical data |
| 55 | 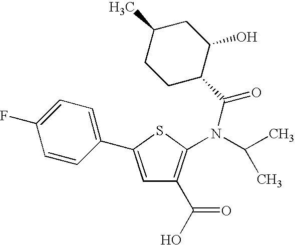 | 16 mg (0.03 mmol) 98A (10 mg (66% of theory)) | 7 mg (0.3 mmol) of LiOH, dioxane:H₂O =2:1, RT, 15 h | HPLC (Method 6): Rₜ = 5.07 min; MS (ESI-neg): m/z = 418 (M − H)⁻ |
| 56 | 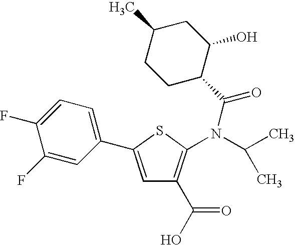 | 32 mg (0.06 mmol) 68A (16 mg (55% of theory)) | 7 mg (0.3 mmol) of LiOH, dioxane:H₂O =2:1, RT, 15 h | HPLC (Method 1): Rₜ = 5.16 min; MS (ESI-neg): m/z = 436 (M − H)⁻ |
| 57 | 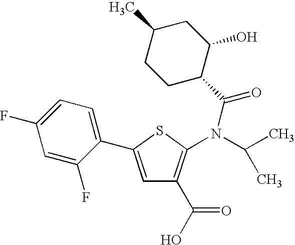 | 30 mg (0.06 mmol) 39A (18 mg (67% of theory)) | 7 mg (0.3 mmol) of LiOH, dioxane:H₂O =20:1, RT, 15 h | HPLC (Method 1): Rₜ = 5.14 min; MS (ESI-pos): m/z = 438 (M + H)⁺ |

### Example 58

### 2-{[(trans-4-Methylcyclohexyl)carbonyl][1-methyl-2-(methylsulfinyl)ethyl]amino}-5-phenylthiophene-3-carboxylic acid

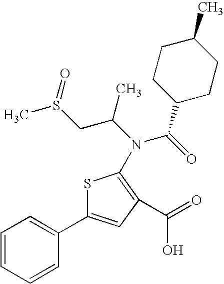

160 mg (0.35 mmol) of the methyl ester from Example 10A are introduced into 10 ml of dioxane and 0.4 ml of water, 37 mg (1.56 mmol) of lithium hydroxide are added, and the mixture is stirred under reflux for 1 h. The mixture is then neutralized with 2 ml of 1N hydrochloric acid, the solvent is removed in vacuo, and the product is purified by preparative HPLC. Yield: 145 mg (93% of theory).

HPLC (Method 6): Rₜ=4.59 min

MS (CI-pos): m/z=448 (M+H)⁺

¹H-NMR (400 MHz, CDCl₃): δ [ppm]=7.71-7.64 (m, 1H); 7.61-7.58 (m, 2H); 7.44-7.33 (m, 3H); 5.04-5.00, 4.83-4.75 (2×m, 1H); 3.59-3.35, 3.10-3.05, 2.91-2.86 (3×m,

2H); 2.80, 2.76, 2.72 (3×s, 3H); 2.27-2.16 (m, 1H); 1.80-1.53 (m, 5H); 1.46-1.25 (m, 5H); 0.78 (d, 3H); 0.75-0.64 (m, 2H).

### Example 59

### 2-{[(trans-4-Methylcyclohexyl)carbonyl[]1-methyl-2-(methylsulfonyl)ethyl]amino}-5-phenylthiophene-3-carboxylic acid

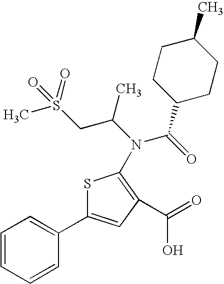

77 mg (0.18 mmol) of the acid from Example 31 are introduced into 5 ml of dichloromethane, 62 mg (0.36 mmol) of meta-chloroperbenzoic acid (70-75% in water) are added in portions, and the mixture is stirred at room temperature. Conversion is not complete after 16 h, and therefore a further 31 mg (0.18 mmol) of meta-chloroperbenzoic acid are added. After a further 30 min, an HPLC check indicates complete conversion, and the mixture is diluted with dichloromethane and added to a saturated thiosulfate solution. The organic phase is separated, dried over sodium sulfate and filtered, and the solvent is removed in vacuo. The product is purified by preparative HPLC. Yield: 66 mg (80% of theory).

HPLC (Method 1): Rₜ=4.9 min

MS (ESI-pos): m/z=464 (M+H)⁺

¹H-NMR (400 MHz, CDCl₃): δ [ppm]=7.71, 7.67 (2×s, 1H); 7.64-7.60 (m, 2H); 7.48-7.36 (m, 3H); 5.12-5.06, 4.97-4.90 (2×m, 1H); 3.84, 3.49 (2×dd, 1H); 3.28, 2.95 (2×dd, 1H); 3.12, 3.09 (2×s, 3H); 0.79 (d, 3H); 0.77-0.67 (m, 2H). (2:1 mixture of isomers)

### Example 60

### 2-{(1,1-Dioxidotetrahydro-2H-thiopyran-3-yl)[(trans-4-methylcyclohexyl)carbonyl]amino}-5-phenylthiophene-3-carboxylic acid

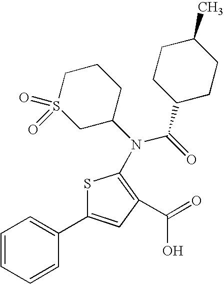

50 mg (0.11 mmol) of the acid from Example 45 are introduced into 5 ml of dichloromethane, 56 mg (0.23 mmol) of meta-chloroperbenzoic acid (70-75% in water) are added in portions, and the mixture is stirred at room temperature. After 15 min, an HPLC check indicates complete conversion, and the mixture is diluted with dichloromethane and added to a saturated thiosulfate solution. The organic phase is separated, dried over sodium sulfate and filtered, and the solvent is removed in vacuo. The product is purified by preparative HPLC. Yield: 49 mg (91% of theory).

HPLC (Method 6): Rₜ=5.00 min

MS (ESI-pos): m/z=476 (M+H)⁺

¹H-NMR (400 MHz, DMSO-D₆): δ [ppm]=7.80 (2×s, 1H); 7.74 (d, 2H); 7.47 (t, 2H); 7.39 (t, 1H); 5.01-4.86 (m, 1H); 3.16-2.97 (m, 3H); 2.18-1.99 (m, 2H); 1.87-1.56 (m, 6.5H); 1.53-1.43 (m, 1H); 1.33-1.24 (m, 3H); 1.03 (d, 0.5H); 0.75 (d, 3H); 0.68-0.57 (m, 2H) (according to NMR, the substance is in the form of an approx. 1:1 mixture of isomers).

### B. Assessment of the Physiological Activity

Abbreviations:

| | |
|---|---|
| DMSO | Dimethylsulfoxide |
| DMEM | Dulbecco's modified Earle's medium |
| FCS | Fetal calf serum |
| G418 | Geneticin |
| EC₅₀ | Effective concentration 50 |
| IC₅₀ | Inhibitory concentration 50 |
| CC₅₀ | Cytotoxic concentration 50 |
| Pen | Penicillin |
| Strep | Streptomycin |
| min. | Minutes |
| NEAA | Nonessential amino acids |
| PBS | Phosphate-buffered saline (phosphate buffer) |
| h | Hours |
| sec. | Seconds |
| RT | Room temperature |

Products Used:

| Product | Company | Catalogue No. |
|---|---|---|
| Escherichia coli BL21 (DE3) | Novagen | 71252 |
| PET21b DNA | Novagen | 69741-3 |
| PCR Primers (5B21AAUP1, 5B21AAD1, | Sigma ARK | — |
| 5B21AAUP1a, 5B21AAD1a) | | |
| β-Mercaptoethanol | Sigma | M6250 |
| LB Medium | Becton | 257243 |
| | Dickinson | |
| Ampicillin | Biomol | 01503 |
| Carbenicillin | Biomol | 50195 |
| IPTG | Biomol | 05684 |
| Ni-NTA | Qiagen | 30210 |
| Imidazole | Sigma | I2399 |
| n-Dodecyl maltoside | Sigma | D4641 |
| poly(A) | Amersham | 27-4110-01 |
| oligo(U)12 | Eurogentec | — |
| UTP | Roche | 1140949 |
| [3H]-UTP | Perkin Elmer | NET380 |
| Rnasin (recombinant) | Promega | N2515 |
| EDTA | Sigma | E5134 |
| DTT | Sigma | D9779 |
| BSA Rnase-free | Roche | 711454 |
| Trichloroacetic acid | Sigma | 490-10 |
| Sodium pyrophosphate | Sigma | S6422 |
| GF/C Filter Multi Screen | Millipore | MAFCN0B10 |
| Liquid Scintillation Cocktails Ultima | Perkin Elmer | 6013117 |
| Gold XR | | |

The in vitro effect of the compounds of the invention can be shown in the following assays:

1. Measurement of the Activity of Substances in a Cellular HCV-RNA Replication Assay (“Replicon System”)

Hepatitis C cannot as of yet be replicated reproducibly with high titres in cell culture. The activity of substances is therefore detected in the so-called replicon system. This comprises parts of the HCV genome or complete HCV genomes which are transferred into cell lines (here HuH-7 cells) of human origin. It is possible by insertion of a selection marker to obtain stable cell lines which, under selection pressure, replicate genomic or subgenomic RNA of HCV [Lohmann et al., Science 285, 110-113 (1999); Blight et al., Science 290, 1972-1974 (2000)]. The HuH5-2 cells used here harbour a selectable, luciferase-carrying, cell culture-adapted replicon as described in EP 1 043 399. The cells are cultured in Dulbecco's modified Earle's medium (DMEM) with 10% fetal calf serum (FCS), 1% Pen/Strep, 1% NEAA, 1% L-glutamine and 250 μg/ml Geneticin (G418). To carry out the assays described below, the cells are initially trypsinized and resuspended with the DMEM medium described above without G418. Depending on the assay, the cells are seeded in 24-well or 384-well plates. Depending on the reading or assay method used, transparent or white assay plates coated for cell culture are used.

a) Preparation of the Test Substances:

The test compounds are made up as 50 mM stock solution in DMSO. To determine the EC₅₀ values, the substances subsequently undergo a serial two-fold dilution in DMEM. The dilutions are transferred as doublets to the cell culture plates. The trypsinized cells which have been resuspended in medium are then added. The final concentration of the test substances in the cell culture wells is for example 300 μM to 0.0001 μM. Interferon-alpha serves as reference substance in concentrations of 60 IU/ml to 1 IU/ml. Antimycin-alpha serves as cytotoxicity control in concentrations of 2 μM to 0.03,M. Untreated cells serve as reference. The plates are then incubated at 37° C. under 5% CO₂ for 4-5 days. The various measurements and the quantification of the HCV replicon RNA then take place.

b) Cytotoxicity Test (Visual):

The cytotoxicity of the test substances on HUH5-2 cells is assessed by setting up the above assay in a transparent cell culture plate. Qualitative evaluation takes place visually under the microscope.

c) Alamar Blue Test (Quantitative Cytotoxicity Test):

Alamar Blue is a water-soluble redox indicator which is reduced as a function of the metabolic activity of the cells to be investigated. The Alamar Blue test is used as quantitative cytotoxicity assay. For this purpose, the cells are seeded with the appropriate test substances (see above) in white 384-well cell culture plates and incubated correspondingly at 37° C. under 5% CO₂ for 4 days. 4 to 6 hours before the actual measurement, 5 μl of Alamar Blue are added per well. The fluorescence is then measured at an emission wavelength of 544 nm and at an extinction wavelength of 590 nm. If this test is to be followed by chemiluminescence measurement (see below) on the same plate, the dye solution is aspirated off from the cells and the latter are subsequently washed once with PBS. The PBS is likewise aspirated off again from the cells.

d) Measurement of the HCV-RNA Amount by Determining the Activity of a Reporter Gene:

A reporter gene is introduced into the HCV replicon HuH5-2, in this case the gene for the luciferase enzyme of Photinus pyralis. After the addition of the luciferase reagent (20 mM Tris/HCl, 20 mM tricine, 2.67 mM MgSO₄, 0.1 mM EDTA, 33.3 mM DTT, 0.27 mM coenzyme A, 0.47 mM luciferin, 0.53 mM ATP, pH 7.8) to the cells, the chemiluminescence is measured in a luminometer. Normally, the photons are measured in a period of from 10 sec to 60 sec.

The following data can be determined from the assay plates:
CC₅₀=substance concentration in μM with which the Alamar Blue fluorescence decreases by 50% compared with the untreated control;
EC₅₀=substance concentration with which the luciferase activity decreases by 50% compared with the untreated replicon control;
SI (selectivity index)=CC₅₀/EC₅₀.

e) Measurement of the Activity of Substances by Direct Measurement of the Amount of HCV Genome:

Cells which replicate subgenomic HCV-RNA are grown as described above in DMEM/10% FCS without addition of Geneticin in 24-well cell culture plates. When the cells are in the logarithmic growth phase, substance is mixed into the medium in suitable dilution. The final concentrations are for example 100 μM and 30 μM, and dilutions thereof. After incubation for 4 to 5 days, the medium is discarded. The cells are detached from the cell culture plate with the aid of trypsin and taken up in 100 μl of phosphate buffer (PBS). The cells are divided, one part being investigated for their content of HCV-RNA with the aid of quantitative PCR, and the other part of the cells being investigated with the aid of luciferase activity detection (Bright Glow Kit, Promega, procedure in accordance with the manufacturer's information). Resulting values are evaluated by curve analyses (sigmoidal dose-response curves with variable curve shape; GraphPad Prism version 3.02 for Windows, GraphPad Software Inc.), and the effective concentration with which a 50% inhibition (EC₅₀) is achieved is determined. Untreated cells are used for comparison. Total cellular RNA is isolated from the remaining cells to be investigated with the aid of the Rneasy Mini Kit (Qiagen, Order No. 74104) in accordance with the manufacturer's information. Elution takes place in 30-50 μl of Rnase-free water. The RNA is stored at −80° C. TaqMan® assays (Applied Biosystems) are used to determine the amount of HCV-RNA contained. The primers and gene probes used bind to the conserved 5′-untranslated region of the viral genome (primer for the coding DNA strand: aatgcctggagatttgggc; primer in opposite direction: tttcgcgacccaacactactc; gene probe: 6-carboxyfluorescin-tgcccccgagactgcatagc-N,N,N′,N′-tetramethyl-6-carboxyrhodamine). To standardize the sample employed, the expression of a gene intrinsic to the cells is determined (TaqMan Ribosomal RNA Control Reagents, Applied Biosystems P/N 4308329). The kit used for the reaction is the Platinum® Quantitative RT-PCR Thermoscript™ one step system from Invitogen (Order No. 12267-019). The reaction takes place in a final volume of 25 μM with 1 μl sample volume. The reaction conditions are: incubation at 50° C. for 30 min, then incubation at 95° C. for 5 min. This step is followed by the actual amplification phase with 40 repetitions of the following steps: incubation at 95° C. for 15 sec followed by incubation at 60° C. for 1 min. Measurement and evaluation took place in an Applied Biosystems Abi Prism 7700 sequence detection instrument. The resulting CT values from the reactions for the target gene (here: HCV) and the cellular reference gene (here: 18s RNA) are used to calculate the relative expression as described in: Abi Prism 7700 Sequence Detection System User Bulletin #2: Relative Quantification of Gene expression (P/N 4303859).

TABLE A
(HCV-RNA replication assay)

| Example No. | EC₅₀ [μM] | SI |
|---|---|---|
| 1 | 1.0 | >50 |
| 19 | 0.4 | >100 |
| 30 | 1.3 | >38 |
| 35 | 2.8 | >18 |
| 42 | 0.5 | >100 |
| 49 | 0.8 | >63 |
| 54 | 0.18 | >277 |

2. Determination of Activity of the RNA-Dependent RNA Polymerase (NS5B) of Hepatitis C Virus in the Presence and Absence of Test Substances

Starting from the plasmid pBac5B-Chis (Lohmann V, Koerner F, Herian U and Bartenschlager R, J. Virol. 71 (1997) 8461-8428) which comprises the complete DNA sequence of a recombinant NS5B gene from the hepatitis C virus of genotype 1b, a polymerase chain reaction (PCR) is used to amplify a DNA sequence (Sambrock J and Russel D W, Cold Spring Harbor Press, New York (2001)) which codes for an NS5B protein truncated at the carboxyl terminus by 21 amino acids. It is possible by using the PCR primers 5B21AAUP1 (5′-AATTGCTAGCATGTCCTACACATGGACAGGCGCCCTGA-3′) and 5B21 AAD1 (5′-TATACTCGAGGCGGGGTCGGGCACGAGACAGGCT-3′) to clone the PCR product into the T7 expression vector pET21b (Novagen) via the recognition cleavage sites for the restriction endonucleases NheI and XhoI. An NS5B protein which comprises amino acids 2420 to 2989 of the HCV precursor polyprotein and a poly-histidine fusion at the carboxyl terminus is expressed with the plasmid pET21NS5Bt constructed in this way. Expression and purification of such a recombinant NS5B variant in the heterologous host Escherichia coli BL21 (DE3) (Novagen) has already been described several times (Ferrari E, Wright-Minogue J, Fang J W S, Baroudy B M, Lau J Y N and Hong Z, J. Virol. 73 (1999) 1649-1654; Tomei L, Vitale R L, Incitti I, Serafini S, Altamura S, Vitelli A und DeFrancesco R, J. Gen. Virol. 81 (2000) 759-767). BL21 (DE3) cells transformed with the plasmid pET21NS5Bt are shaken in LB medium (plus 100 μg/ml ampicillin) at 37° C. until the optical density O.D. (600 nm)=0.6 and then induced with 0.5 mM IPTG (isopropyl beta-D-thiogalactopyranoside). To prevent cell inclusion bodies, expression takes place at 20° C. to 25° C. for 4 hours. The cell sediments obtained by centrifugation (20 min; 5000×g; 4° C.) are concentrated to an O.D. (600 nm)=50 to 500 in cell lysis buffer (50 mM NaH₂PO₄; 5 mM Tris-HCl (tris(hydroxymethyl)aminomethane) pH 8.0; 25 mM imidazole; 10 mM MgCl₂; 500 mM NaCl; 0.1% (v/v) β-mercaptoethanol; 1 mM EDTA (ethylenediamine-N,N,N′,N′-tetraacetate); 10% (v/v) glycerol; 1 tablet/50 ml Complete (Roche); 10 μg/ml DNase I) and then disrupted with ultrasound (10×30 s; 200 Watt; 0° C.). The soluble protein fraction, which is separated from the insoluble protein fraction by centrifugation (20 min; 10 000×g; 4° C.), is sterilized by filtration (<0.45 μm) and loaded onto an Ni-NTA column (nickel-nitrilotriacetic acid, Qiagen) equilibrated with cell lysis buffer. Loading of the sample is followed by a washing step with 10 column volumes of cell lysis buffer and subsequently with 20 column volumes of washing buffer (50 mM NaH₂PO₄; 5 mM Tris-HCl pH 8.0; 25 mM imidazole; 10 mM MgCl₂; 500 mM NaCl; 0.1% (v/v) β-mercaptoethanol; 1 mM EDTA; 10% (v/v) glycerol). The protein elution takes place with an imidazole stepped gradient (washing buffer supplemented with 50 mM, 100 mM, 250 mM and 500 mM imidazole) with in each case 5 column volumes per imidazole step. Fractions with a volume of from 1 to 2 ml are collected during the elution and are analysed in an SDS-PAGE. The NS5B-containing fractions are combined and the buffer is changed to storage buffer (25 mM Tris-HCl pH 7.5; 0.3 M NaCl; 10 mM MgCl₂; 5 mM DTT (dithiothreitol); 1 mM EDTA; 0.1% n-dodecyl maltoside; 30% glycerol) with the aid of a PD10 column (Amersham) in accordance with the manufacturer's information and stored at −80° C. An analogous procedure is carried out as mock control with a protein extract obtained from Escherichia coli BL21 (DE3) cells transformed with the empty vector pET21b.

The NS5B activity is detected by carrying out an RNA polymerase-catalysed primer elongation reaction as has already been described (Ferrari et al., 1999; Tomei et al., 2000). This entails a single-stranded, homopolymeric RNA template (poly(rA) (Amersham)) being converted with the aid of RNA primers (oligo(rU)₁₂ (Eurogentec)) and the substrate UTP into a double-stranded RNA duplex. Incorporation of the substrate can be quantified by using radiolabelled UTP, e.g., [³²P]-UTP. As a difference from the majority of published assay formats, tritium-labelled UTP ([³H]-UTP ([5,6-³H]-uridine 5′-triphosphate), Perkin Elmer) is used instead of [³²P]-UTP, as has already been used by Uchiyama et al. (Uchiyama Y, Huang Y, Kanamori H, Uchida M, Doi T, Takamizawa A, Hamakubo T and Kodama T, Hepatol. Res. 23 (2002) 90-97). A reaction mixture contains 6 μg/ml poly(rA), 90 nM oligo(rU)₁₂, 5 μM UTP and 16 μCi/ml [³H]-UTP and in 90 μl of reaction buffer (20 mM Tris-HCl pH 7.5; 25 μM KCl; 5 mM MgCl₂; 1 mM EDTA; 1 mM DTT; 0.01% (w/v) BSA; 0.5 (v/v) DMSO; 100 U/ml RNasin (Promega)). If the effect of substances on the polymerase activity is to be tested, the substance to be tested is added in the desired concentration before adding template, primer and substrate to the reaction mixture. The reaction is started by adding 12.5 nM NS5B protein and is incubated at 30° C. After an incubation time of 120 min, the reaction is stopped with 1 volume of ice-cold stop buffer 1 (0.2 M EDTA; 100 μg/ml calf thymus DNA) and precipitated in 4 volumes of stop solution 2 (10% (w/v) trichloroacetic acid; 0.5% (w/v) sodium pyrophosphate for 30 min on ice. The precipitate is transferred to GF/C filters in a 96-well microtitre plate format (Millipore) and washed 3 times with washing solution 1 (1% (w/v) trichloroacetic acid; 0.1% (w/v) sodium pyrophosphate) and twice with 95% (v/v) ethanol. Scintillation fluid (Liquid Scintillation

Cocktails Ultima Gold XR, Packard Instruments) is added to the dried filters, which are incubated for a further 30 min and then read with a Microbeta counter (1450 Microbeta Plus, Wallac) in accordance with the manufacturer's information. The incorporated amount of [³H]-UTP and the measured CPM (counts per minute) respectively a measure of the activity of the NS5B polymerase. The IC₅₀ values are determined by plotting the relative incorporation of [³H]-UTP against the concentration of the test substance employed in a dose-response curve. The IC₅₀ values are determined with the aid of the GraphPad Prism 3.02 analysis software (GraphPad Software, INC) using the function “Sigmoidal dose-response curve with variable slope”. The relative incorporation of [³H]-UTP without addition of a test substance is used as reference.

C. Exemplary Embodiments of Pharmaceutical Compositions

The compounds of the invention can be converted into pharmaceutical preparations in the following ways:

Tablet:

Composition:
100 mg of the compound of Example 1, 50 mg of lactose (monohydrate), 50 mg of maize starch (native), 10 mg of polyvinylpyrolidone (PVP 25) (BASF, Ludwigshafen, Germany) and 2 mg of magnesium stearate.
Tablet weight 212 mg. Diameter 8 mm, radius of curvature 12 mm.

Production:

A mixture of active ingredient, lactose and starch is granulated with a 5% solution (m/m) of PVP in water. The granules are dried and then mixed with the magnesium stearate for 5 min. This mixture is compressed with a conventional tablet press (see above for format of the tablet). A compressive force of 15 kN is used as guideline for the compression.

Suspension which can be Administered Orally:

Composition: 1000 mg of the compound of Example 1, 1000 mg of ethanol (96%), 400 mg of Rhodigel (xanthan gum from FMC, Pennsylvania, USA) and 99 g of water.
10 ml of the oral suspension are equivalent to a single dose of 100 mg of the compound of the invention.

Production:

The Rhodigel is suspended in ethanol, and the active ingredient is added to the suspension. The water is added while stirring. The mixture is stirred for about 6 h until the swelling of the Rhodigel is complete.

Solution which can Administered Orally:

Composition:
500 mg of the compound of the invention, 2.5 g polysorbate and 97 g of polyethylene glycol 400.20 g of the oral solution are equivalent to a single dose of 100 mg of the compound of the invention.

Production:

The compound of the invention is suspended in the mixture of polyethylene glycol and polysorbate with stirring. Stirring is continued until the compound of the invention is completely dissolved.

I.V. Solution:

The compound of the invention is dissolved in a concentration below saturation solubility in a physiologically tolerated solvent (e.g., isotonic saline solution, glucose solution 5% and/or PEG 400 solution 30%). The solution is sterilized by filtration and dispersed into sterile and pyrogen-free injection containers.

1. A compound of formula 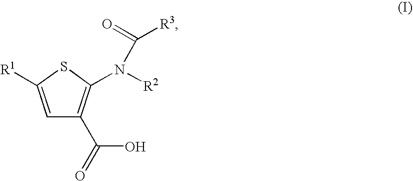
in which
R¹ is selected from the group consisting of (C₃-C₆)-alkyl, (C₃-C₇)-cycloalkyl, 5- to 7-membered heterocyclyl, phenyl and 5- or 6-membered heteroaryl,
whereby phenyl, cycloalkyl, heterocyclyl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, aminothiocarbonyl, hydroxymethyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkylsulfoxyl, (C₁-C₆)-alkoxycarbonyl, (C₁-C₆)-alkylaminocarbonyl, (C₁-C₆)-alkylaminothiocarbonyl and (C₁-C₆)-alkylcarbonylamino,
R² is selected from the group consisting of (C₁-C₆)-alkyl, (C₃-C₇)-cycloalkyl, 5- to 7-membered heterocyclyl and benzyl,
whereby alkyl, cycloalkyl, heterocyclyl and benzyl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkylsulfoxyl, (C₁-C₆)-alkoxycarbonyl, (C₁-C₆)-alkylaminocarbonyl, (C₁-C₆)-alkylcarbonylamino, 5- to 7-membered heterocyclyl, optionally alkyl-substituted (C₃-C₇)-cycloalkylaminocarbonyl and optionally alkyl-substituted 5- to 7-membered heterocyclylcarbonyl,
in which alkyl may be substituted with 1 to 2 substituents, whereby said substituents are selected independently of one another from the group consisting of hydroxy, amino, (C₁-C₆)-alkylamino, (C₁-C₆)-alkylsulfoxyl and (C₁-C₆)-alkoxycarbonyl,
R³ is selected from the group consisting of (C₃-C₇)-cycloalkyl, 5- to 7-membered heterocyclyl, (C₆-C₁₀)-aryl, 5- to 7-membered heteroaryl, —CH₂—R⁴ and —CH₂—CH₂—R⁵,
whereby cycloalkyl, heterocyclyl, aryl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkylthio, (C₁-C₆)-alkylcarbonyl, (C₁-C₆)-alkylsulfonyl, (C₁-C₆)-alkoxycarbonyl, (C₆-C₁₀)-aryloxycarbonyl, (C₁-C₆)-alkylaminocarbonyl, (C₁-C₆)-alkylcarbonylamino, —OR⁶ and —NR⁷R⁸,
in which alkyl may be substituted with 1 to 3 substituents, whereby said substituents are selected independently of one another from the group consisting of halogen, hydroxy, amino, hydroxycarbonyl, aminocarbonyl, (C₁-C₆)-alkoxy, (C₁-C₆)-alkylamino, (C₁-C₆)-alkylthio, phenyl, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
in which phenyl in turn may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
whereby
R⁶ and R⁷ are independently of one another selected from the group consisting of (C₁-C₆)-alkyl, (C₃-C₇)-cycloalkyl, 5- to 7-membered heterocyclyl, benzyl, (C₆-C₁₀)-aryl and 5- or 6-membered heteroaryl,
whereby alkyl, cycloalkyl, heterocyclyl, benzyl, aryl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
R⁸ is selected from the group consisting of hydrogen, (C₁-C₆)-alkyl and (C₃-C₇)-cycloalkyl,
or
R⁷ and R⁸ with the nitrogen atom to which they are bonded may form a 5-to 7-membered heterocycle,
R⁴ and R⁵ are independently of one another selected from the group consisting of (C₃-C₇)-cycloalkyl, 5- to 7-membered heterocyclyl, (C₆-C₁₀)-aryl and 5- to 7-membered heteroaryl,
whereby cycloalkyl, heterocyclyl, aryl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino;
or a salt thereof, a solvate thereof or a solvate of a salt thereof;

2. The compound of claim 1, whereby
R¹ is selected from the group consisting of piperidinyl, piperazinyl, phenyl, pyridyl and thienyl,
whereby piperidinyl, piperazinyl, phenyl, pyridyl and thienyl may be substituted with 1 to 2 substituents, whereby said substituents are selected independently of one another from the group consisting of fluorine, chlorine, cyano, nitro, trifluoromethyl, trifluoromethoxy, methyl, methoxy, (C₁-C₄)-alkylamino, (C₁-C₄)-alkylcarbonyl, methylsulfonyl, (C₁-C₄)-alkoxycarbonyl and (C₁-C₄)-alkylaminothiocarbonyl,
R² is selected from the group consisting of branched (C₃-C₅)-alkyl, cyclobutyl, cyclopentyl, cyclohexyl, pyrrolidinyl, tetrahydro-2H-pyranyl, piperidinyl, tetrahydro-2H-thiopyranyl, oxidotetrahydro-2H-thiopyranyl and 1,1-dioxidotetrahydro-2H-thiopyranyl,
whereby alkyl may be substituted with 1 to 2 substituents, whereby said substituents are selected independently of one another from the group consisting of methoxy, methylthio, methylsulfonyl, methylsulfoxyl or methoxycarbonyl,
R³ is selected from the group consisting of cyclohexyl and phenyl,
whereby cyclohexyl and phenyl may be substituted with 1 to 2 substituents, whereby said substituents are selected independently of one another from the group consisting of halogen, hydroxy, (C₁-C₃)-alkyl and —OR⁶,
whereby
R⁶ is (C₁-C₃)-alkyl.

3. The compound of claim 1, whereby
R¹ is selected from the group consisting of piperidinyl, phenyl and pyridyl,
whereby phenyl and pyridyl may be substituted with 1 to 2 substituents, whereby said substituents are selected independently of one another from the group consisting of fluorine, chlorine, cyano, nitro, trifluoromethyl, trifluoromethoxy, methyl, methoxy and methylsulfonyl,
and
whereby piperidinyl may be substituted with 1 to 2 substituents, whereby said substituents are selected independently of one another from the group consisting of fluorine, cyano, trifluoromethyl, trifluoromethoxy, methyl, methoxy and methylsulfonyl,
R² is selected from the group consisting of branched (C₃-C₅)-alkyl, cyclobutyl, cyclopentyl, cyclohexyl, pyrrolidinyl, tetrahydro-2H-pyranyl, piperidinyl, tetrahydro-2H-thiopyranyl, oxidotetrahydro-2H-thiopyranyl and 1,1-dioxidotetrahydro-2H-thiopyranyl,
whereby alkyl may be substituted with 1 to 2 substituents, whereby said substituents are selected independently of one another from the group consisting of methoxy, methylthio, methylsulfonyl, methylsulfoxyl or methoxycarbonyl,
R³ is cyclohexyl,
whereby cyclohexyl may be substituted with 1 to 2 substituents, whereby said substituents are selected independently of one another from the group consisting of hydroxy and methyl.

4. The compound of claim 1, whereby R² is selected from the group consisting of cyclobutyl, cyclopentyl and cyclohexyl.

5. The compound of claim 1, whereby R² is selected from the group consisting of pyrrolidinyl, tetrahydro-2H-pyranyl, piperidinyl, tetrahydro-2H-thiopyranyl, oxidotetrahydro-2H-thiopyranyl and 1,1-dioxidotetrahydro-2H-thiopyranyl.

6. The compound of claim 1, whereby R³ is cyclohexyl, whereby cyclohexyl may be substituted with 1 to 2 substituents, whereby said substituents are selected independently of one another from the group consisting of hydroxy and methyl.

7. A process for preparing a compound of formula (I) according to claim 1, whereby a compound of formula 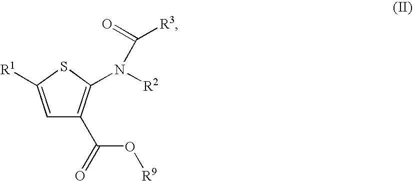
in which
R¹, R² and R³ have the meaning indicated in claim 1, and
R⁹ is alkyl
is reacted with a base.

8. The process of claim 7, whereby R⁹ is selected from the group consisting of methyl, ethyl and tert-butyl.

9. A process for preparing a compound of formula (I) according to claim 1, whereby a compound of formula 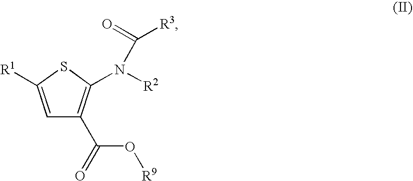
in which
R¹, R² and R³ have the meaning indicated in claim 1, and
R⁹ is alkyl
is reacted with an acid.

10. The process of claim 9, whereby R⁹ is selected from the group consisting of methyl, ethyl and tert-butyl.

11. The compound of claim 1 for the treatment of diseases.

12. The compound of claim 1 for the prophylaxis of diseases.

13. The compound of claim 1 for the treatment and prophylaxis of diseases.

14. A method for the production of a medicament for the treatment of diseases using a compound of claim 1.

15. A method for the production of a medicament for the prophylaxis of diseases using a compound of claim 1.

16. A method for the production of a medicament for the treatment and prophylaxis of diseases using a compound of claim 1.

17. A method for the production of a medicament for the treatment of viral infections using a compound of claim 1.

18. A method for the production of a medicament for the prophylaxis of viral infections using a compound of claim 1.

19. A method for the production of a medicament for the treatment and prophylaxis of viral infections using a compound of claim 1.

20. The method of claim 17, whereby said viral infections are infections with a virus selected from the group consisting of the hepatitis C virus and other representatives of the group of Flaviviridae.

21. The method of claim 18, whereby said viral infections are infections with a virus selected from the group consisting of the hepatitis C virus and other representatives of the group of Flaviviridae.

22. The method of claim 19, whereby said viral infections are infections with a virus selected from the group consisting of the hepatitis C virus and other representatives of the group of Flaviviridae.

23. A medicament comprising a compound of claim 1, in combination with a further active ingredient.

24. A medicament comprising a compound of claim 1 in combination with an inert, non-toxic, pharmaceutically acceptable excipient.

25. The medicament according to claim 24 for the treatment of viral infections.

26. The medicament according to claim 24 for the prophylaxis of viral infections.

27. The medicament according to claim 24 for the treatment and prophylaxis of viral infections.

28. A method for controlling viral infections in humans or animals by administering an antivirally effective amount of at least one compound formula (I): 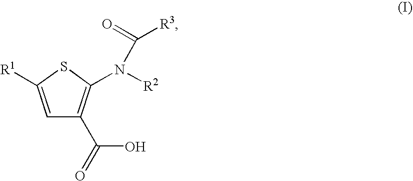
in which
R¹ is selected from the group consisting of (C₃-C₆)-alkyl, (C₃-C₇)-cycloalkyl, 5- to 7-membered heterocyclyl, phenyl and 5- or 6-membered heteroaryl,
whereby phenyl, cycloalkyl, heterocyclyl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, aminothiocarbonyl, hydroxymethyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkylsulfoxyl, (C₁-C₆)-alkoxycarbonyl, (C₁-C₆)-alkylaminocarbonyl, (C₁-C₆)-alkylaminothiocarbonyl and (C₁-C₆)-alkylcarbonylamino,
R² is selected from the group consisting of (C₁-C₆)-alkyl, (C₃-C₇)-cycloalkyl, 5- to 7-membered heterocyclyl and benzyl,
whereby alkyl, cycloalkyl, heterocyclyl and benzyl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkylsulfoxyl, (C₁-C₆)-alkoxycarbonyl, (C₁-C₆)-alkylaminocarbonyl, (C₁-C₆)-alkylcarbonylamino, 5- to 7-membered heterocyclyl, optionally alkyl-substituted (C₃-C₇)-cycloalkylaminocarbonyl and optionally alkyl-substituted 5- to 7-membered heterocyclylcarbonyl,
in which alkyl may be substituted with 1 to 2 substituents, whereby said substituents are selected independently of one another from the group consisting of hydroxy, amino, (C₁-C₆)-alkylamino, (C₁-C₆)-alkylsulfoxyl and (C₁-C₆)-alkoxycarbonyl,
R³ is selected from the group consisting of (C₃-C₇)-cycloalkyl, 5- to 7-membered heterocyclyl, (C₆-C₁₀)-aryl, 5- to 7-membered heteroaryl, —CH₂—R⁴ and —CH₂—CH₂—R⁵,
whereby cycloalkyl, heterocyclyl, aryl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkylthio, (C₁-C₆)-alkylcarbonyl, (C₁-C₆)-alkylsulfonyl, (C₁-C₆)-alkoxycarbonyl, (C₆-C₁₀)-aryloxycarbonyl, (C₁-C₆)-alkylaminocarbonyl, (C₁-C₆)-alkylcarbonylamino, —OR⁶ and —NR⁷R⁸,
in which alkyl may be substituted with 1 to 3 substituents, whereby said substituents are selected independently of one another from the group consisting of halogen, hydroxy, amino, hydroxycarbonyl, aminocarbonyl, (C₁-C₆)-alkoxy, (C₁-C₆)-alkylamino, (C₁-C₆)-alkylthio, phenyl, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
in which phenyl in turn may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
whereby
R⁶ and R⁷ are independently of one another selected from the group consisting of (C₁-C₆)-alkyl, (C₃-C₇)-cycloalkyl, 5- to 7-membered heterocyclyl, benzyl, (C₆-C₁₀)-aryl and 5- or 6-membered heteroaryl,
whereby alkyl, cycloalkyl, heterocyclyl, benzyl, aryl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
R⁸ is selected from the group consisting of hydrogen, (C₁-C₆)-alkyl and (C₃-C₇)-cycloalkyl,
or
R⁷ and R⁸ with the nitrogen atom to which they are bonded may form a 5- to 7-membered heterocycle,
R⁴ and R⁵ are independently of one another selected from the group consisting of (C₃-C₇)-cycloalkyl, 5- to 7-membered heterocyclyl, (C₆-C₁₀)-aryl and 5- to 7-membered heteroaryl,
whereby cycloalkyl, heterocyclyl, aryl and heteroaryl may be substituted with 1 to 3 substituents, whereby said substituents are selected independently of one another from the group consisting of halogen, cyano, hydroxy, amino, nitro, trifluoromethyl, trifluoromethoxy, hydroxycarbonyl, aminocarbonyl, (C₁-C₆)-alkyl, (C₁-C₆)-alkoxy, (C₁-C₆)-alkylamino, (C₁-C₆)-alkylthio, (C₁-C₆)-alkylcarbonyl, (C₁-C₆)-alkylsulfonyl, (C₁-C₆)-alkoxycarbonyl, (C₁-C₆)-alkylaminocarbonyl and (C₁-C₆)-alkylcarbonylamino,
or a salt thereof, a solvate thereof or a solvate of a salt thereof.

29. The method of claim 28, wherein said compound is in the form of a medicament.

30. The method of claim 29, whereby said medicament comprises a further active ingredient.

31. The method of claim 29, whereby said medicament comprises an inert, non-toxic, pharmaceutically acceptable excipient.
